(19) United States
(12) Patent Application Publication
(10) Pub. No.: US 2025/0082747 A1
(43) Pub. Date: Mar. 13, 2025

(54) PORCINE CORONAVIRUS VACCINES

(71) Applicant: Boehringer Ingelheim Animal Health USA Inc., Duluth, GA (US)

(72) Inventors: Scott ACKERMAN, Huxley, IA (US); Joseph Ralph HERMANN, Waukee, IA (US); Luis Alejandro HERNANDEZ, Story City, IA (US); Lea Ann HOBBS, Nevada, IA (US); Arun V. IYER, Ames, IA (US); Sean O'CONNER, Ankeny, IA (US); Abby Rae PATTERSON, Story City, IA (US); Eric Martin VAUGHN, Ames, IA (US); Joseph Gilbert VICTORIA, Ames, IA (US)

(21) Appl. No.: 18/893,431

(22) Filed: Sep. 23, 2024

Related U.S. Application Data
(63) Continuation of application No. 16/481,010, filed on Jul. 25, 2019, now Pat. No. 12,150,983.
(60) Provisional application No. 62/452,026, filed on Jan. 30, 2017.

(51) Int. Cl. A61K 39/215; A61K 39/00; A61P 31/14; C12N 7/00

(52) U.S. Cl. CPC A61K 39/215; A61P 31/14; C12N 7/00; A61K 2039/5252; A61K 2039/552; A61K 2039/55566; C12N 2710/14043; C12N 2710/14071; C12N 2770/20034; C12N 2770/20071

(57) ABSTRACT

The present invention relates to a vaccine for protecting a pig against diseases associated with corona virus infection including porcine epidemic diarrhea virus (PEDV) and/or porcine deltacorona virus (PDCoV). The vaccine commonly includes inactivated/killed PEDV (e.g., chemically inactivated PED virus), and/or recombinant PEDV antigen, and/or an adjuvant inactivated/killed PDCoV (e.g., chemically inactivated PDCoV virus), and/or recombinant PDCoV antigen and an adjuvant. Methods for protecting pigs against diseases associated with PEDV and/or PDCoV and methods of producing the porcine epidemic diarrhea virus and/or porcine deltacorona virus vaccine are also provided.

### CROSS-REFERENCE TO RELATED APPLICATIONS

This application is a continuation of U.S. patent application Ser. No. 16/481,010, filed Jul. 25, 2019, which is a national stage application of International Application No. PCT/US2018/015507, filed Jan. 26, 2018, which claims the benefit of U.S. Provisional Application No. 62/452,026, filed Jan. 30, 2017, the entire contents of which are hereby incorporated by reference herein.

### INCORPORATION BY REFERENCE

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The Sequence Listing associated with this application is provided in XML format in lieu of a paper copy, and is hereby incorporated by reference into the specification. The name of the XML file containing the Sequence Listing is 10-0176-US-3-SL.xml. The XML file is 375,206 bytes; it was created on 23 Sep. 2024; and it is being submitted electronically via EFS-Web, concurrent with the filing of the specification.

### BACKGROUND OF THE INVENTION

### Field of the Invention

The present invention relates to immunogenic compositions that protect swine from disease caused by porcine epidemic diarrhea virus (PEDV) vaccine specific and porcine Deltacoronavirus (PDCoV), and combination vaccines providing both PEDV and PDCoV antigens. Due to the high mortality (up to 100%) in less than 10 day old piglets, the prevalent enteric diseases, often present together, are of major economic concern to the U.S. swine industry.

### Description of the Related Art

The porcine epidemic diarrhea virus is an enveloped, positive-sense single-stranded RNA virus that causes acute diarrhea, vomiting, and dehydration in pigs. It was first identified in Europe but has become increasingly problematic in many Asian countries, including Korea, China, Japan, the Philippines, and Thailand. In April of 2013, PEDV emerged in U.S. swine in the Midwest, swiftly spreading across the country. By October 2013, PEDV was detected in swine herds in 18 States. The economic impact of PEDV infection has already been substantial. North American isolates of PEDV have been identified (Huang, et al. 2013; Stevenson et al. 2013), however no fully licensed vaccine is commercially available in the United States. Accordingly, there is a continuing need to develop vaccines capable of protecting pigs against disease associated with PEDV. It would be advantageous to develop a vaccine that is effective against emerging North American PEDV strains which could be administered via a mucosal route (oral or intranasal) as well as via parenteral methods (e.g., intramuscularly, subcutaneously or intravenously).

PEDV is a member of the subfamily Coronavirinae of genus Alphacoronavirus (Bridgen et al. 1993) and was first identified in England in 1971 and later in other countries, such as Belgium, China, Hungary, Italy, Japan, Korea, and Thailand (Oldham J. 1972; Pensaert and De Bouck P. 1978; Chen et al. 2008; Nagy et al. 1996; Martelli et al. 2008; Takahashi et al. 1983; Chae et al. 2000; and Puranaveja et al. 2009). Other members of this family include Porcine Respiratory Coronavirus (PRCV), Hemagglutinating Encephalomyelitis Coronavirus (PHE), and Transmissible Gastroenteritis Virus (TGEV). Although PEDV and TGEV viruses are related and the clinical signs are very similar, there is no immune cross-protection.

PEDV is an enveloped virus possessing approximately a 28 kb, positive-sense, single stranded RNA genome, with a 5′ cap and a 3′ polyadenylated tail. (Pensaert and De Bouck P. 1978). The genome comprises a 5′ untranslated region (UTR), a 3′ UTR, and at least seven open reading frames (ORFs) that encode four structural proteins (spike(S), envelope (E), membrane (M), and nucleocapsid (N)) and three non-structural proteins (replicases 1a and 1b and ORF3); these are arranged on the genome in the order 5′-replicase (1a/1b)-S-ORF3-E-M-N-3′ (Oldham J. 1972; and Bridgen et al. 1993). The first three emergent North American PEDV genomic sequences characterized, Minnesota MN (GenBank: KF468752.1), Iowa IA1 (GenBank: KF468753.1), and Iowa IA2 (GenBank: KF468754.1), have the same size of 28,038 nucleotides (nt), excluding the polyadenosine tail and share the genome organization with the prototype PEDV CV777 strain (GenBank: AF353511.1). These three North American PEDV sequences shared 99.8 to 99.9% nucleotide identities. In particular, strains MN and IA2 had only 11 nucleotide differences across the entire genome.

The PEDV S protein is a type I glycoprotein composed of 1,383 amino acids (aa). The S protein can be divided into S1 (1-789 aa) and S2 (790-1,383 aa) domains based on its homology with S protein of other coronaviruses (Chang et al; 2002; Cruz et al, 1994; Godet, et al 1994; Jackwood et al. 2001; Sturman and Holmes; 1984; and Sun et al. 2008). The S protein in coronaviruses is a surface antigen, where it plays a role in regulating interactions with host cell receptor glycoproteins to mediate viral entry, and stimulating induction of neutralizing antibodies in the natural host. Thus the S glycoprotein is a primary target for the development of effective vaccines against PEDV.

The PEDV M protein is the most abundant envelope component playing an important role in the viral assembly process and also induces antibodies that neutralize the virus. Likewise the PEDV N protein, which binds to virion RNA providing a structural basis for the nucleocapsid, may also be important for induction of cell-mediated immunity (Saif, L. 1993).

The only accessory gene in PEDV is ORF3. While accessory genes are generally maintained in field strains, alteration of ORF3 is thought to influence virulence; cell culture adaptation has been used to alter the ORF3 gene in order to reduce virulence (Song et al. 2003). In fact, through investigation of the ORF3 gene, researchers have charted the emergence of new genogroups of PEDV in immunized swine herds in China since 2006. Phylogenic studies of these strains and the geographical reemergence of PEDV in China have demonstrated that those field strains causing devastating enteric disease differ genetically in ORF3 from the European strains and vaccine strains (Park et al. 2011).

It is well know that different strains of PEDV do exist with varying levels of virulence. During the 1980s and 1990s, PEDV was prevalent throughout Europe, in countries such as Belgium, England, Germany, France, the Netherlands, and Switzerland. The frequency of reported cases in Europe subsequently tapered off and/or the disease caused by PEDV was not of sufficient economic importance to start commercial development of a vaccine (Song and Park 2012). While outbreaks of PEDV have been documented in China since the 1980s, variant strains of PEDV emerging since 2010 associated with large-scale outbreaks of diarrhea have been more acute and severe. Thus the trial of vaccine development was mainly accomplished in Asian countries (Song and Park 2012). Variants emerging since 2010 have been reported as having 80-90% morbidity and 50-90% mortality in suckling piglets (Bi et al. 2012; Pan et al. 2012; and Li et al. 1012). Recent evidence suggests that the emerging virulent forms of PEDV in China may be a result of evolution of the live vaccine strains (Chen et al. 2010).

As an enteric disease affecting the pig's intestine, PEDV spreads via fecal-oral exposure. Contaminated trucks and equipment are frequent sources of infection to naïve animals. The clinical signs of PEDV infection are similar to transmissible gastroenteritis virus (TGEV) infection (Pijpers et al. 1993). In pigs three weeks of age and younger, clinical signs (including acute watery, diarrhea, vomiting, and dehydration) can be seen as soon as 24 hours after PEDV infection leading to 100% mortality. PEDV-infected feeder and grower pigs, as well as sows and boars, can develop diarrhea and vomiting. The animals can also show signs of anorexia and can be lethargic. The full impact on older pigs is yet to be determined, but reduced feed efficiency, additional days to market, and the susceptibility of infected animals to secondary infections is likely. For sows, reduced body condition may negatively impact reproductive performance. Reports have indicated that there are signs that PEDV could become endemic in North American herds, resulting in persistent diarrhea and other challenges.

The gross and histological changes in the gut of animals infected with PEDV are similar in the United States as those observed in China; essentially the virus destroys the villi of a pig's intestine so that there is a failure to absorb nutrients. Huang et al. 2012 reported that animals succumbing to the disease in the Minnesota and Iowa outbreaks had gross pathological lesions confined to the small intestine and that the small intestine was characterized by thin translucent walls distended with yellow fluid. Histological evaluations revealed regions of small intestines with villus blunting and fusion and minimal lymphoblastic infiltration of the villi of the lamia propria.

Huang et al. 2013 characterized three different strains of PEDV from outgoing outbreaks in the United States—one from Minnesota and two from Iowa, designated MN (GenBank accession No: KF468752) and IA1 (GenBank accession No: KF468753) and IA2 (GenBank accession No: KF48754), respectively. Huang's phylogenic survey grouped PEDV strains as falling into two distinct genogroups, designated genogroup 1 (G1) and genogroup 2 (G2). The significant changes in the N-terminal domain (NTD) of the spike gene differentiated genogroup 1 and 2. Huang et al. 2013 suggests that the second deletion region (DR2) in the N-terminal domain (NTD) appears to have a higher degree of antigenic change than DR1, suggesting that the emerging North American strains may be less “antigenically” related to the G1a vaccine strains.

Genogroup 1 includes at least three clusters 1a, 1b, and R. Subgroup 1a includes the early European, Chinese, and Korean isolates, e.g., prototype CV777 strain (Belgium, 1978, GenBank: AF353511.1) and strains LZC (Gansu, China, 2006; GenBank: EF185992) and SM98 (Korea, 1998; GenBank: GU937797.1). Subgroup 1b contains five strains—one from South Korea (the DR13 attenuated vaccine strain, GenBank: JQ023162.1) and the others from China linked by the common “genetic signature” 8-aa deletion in nsp3 and the large ORF3 deletion at the C terminus. Group “R” is associated with recombinants of the other genogroups. However, the newly emergent PEDV strains, including those arising in China since 2010 and in North America since 2013, belong to genogroup G2a. The Chinese strain AH2012 (GenBank accession no: KC210145) and the North American strains share several unique nucleotides changes and are clustered together in genogroup 2a. Nucleotide identity to AH2012 for strains MN and IA2 was 99.6% and for strain IA1 was 99.5%. Researchers have speculated that an AH2012-like virus was possibly transmitted to the eastern China regions and then transported to the United States and is most likely the closest ancestor to the North American strains. Members of the genogroup 2a share only approximately 96.9% similarity to the prototype PEDV strain CV777 of genogroup 1a (Bridgen, et al. 1993; Huang et al. 2013; GenBank: AF353511.1). As such, the attenuated PEDV vaccines based on the historical CV777-derived G1a strains or DR13-derived G1b strains may be antigenically less related to the newly emergent Chinese and North American G2a PEDV strains and therefore may be poor vaccine candidates.

A closely related North American isolate US/Colorado/2013 (GenBank Accession No: KF272920.1) has also been reported by Marthaler et al, 2013. Like the North American isolates above, the complete PEDV genome of CO/13 has a nucleotide identity of 96.5 to 99.5% with other complete PEDV genomes available in GenBank, with the highest nucleotide identity (99.5%) with Chinese strain AH2012 (GenBank Accession No. KC210145). It is a member of the 2a genogroup. Comparison of the complete genome of CO/13 to that of PEDV reference strain CV777, demonstrates that CO/13 contains a 1-nt insertion (at position 48) and deletions of 5 nucleotides in the 5′ UTR (at positions 73 and 83 to 86). This North American virus exhibits increased divergence within S1 at genomic positions 20,696 and 21,125 sharing only 82% nucleotide identity with several insertions/deletions.

Several PEDV vaccines, which differ in their genomic sequence, mode of delivery, and efficacy, have been developed. A cell culture adaptation of the European CV777 strain has been used in Asian countries where the PEDV outbreaks have been severe. These have been in use since the 1990s.

In the early 1980s Japanese researchers isolated a causative PED virus strain 83P-5 from the diarrhea of an infected pig. Kusanagi et al. 1989 isolated and adapted the strain in Vero cells. An attenuated virus vaccine of cell culture adapted PEDV (P-5V) (83P-5) has been used in Japan in sows since 1997. The 100^{th}-passaged 83P-5 strain was licensed for use as an attenuated PEDV vaccine in Japan by Nisseiken Co., Ltd. (Sato et al. 2011). It has been reported that adaptation and attenuation of the 83P-5strain showed mutations in the extra-cellular portion of the S protein with sequence similarity to that of the attenuated DR13 strain (Sato et al. 2011; See Strain 83P-5 Spike gene sequence at 100^{th} passage, GenBank: AB548621.1). Although this later Japanese vaccine is considered efficacious, not all sows were able to pass immunity to their piglets (Usami et al. 1998). The Japanese strains and the European strains are members of genogroup G1a or G1b. As discussed above these attenuated vaccine strains are less related to the divergent North American strains than the newly emergent Chinese strains of genogroup 2a.

Oral vaccination with an attenuated Korean PEDV strain, DR13 (passage level 100) (GenBank: JQ023162.1), a member of genogroup G1b, has been shown to be efficacious as a vaccine. The viral strain was licensed and used as an oral vaccine in South Korea since 2004, and registered and commercialized in the Philippines in 2011 (Song and Park 2012). However, it has been reported that attenuated DR13 does not significantly alter the duration of virus shedding in challenged piglets—an indication that immune protection is incomplete. Moreover, oral immunization with highly attenuated PEDV only conferred protection at very high doses of vaccine (Song and Park 2012).

Other known vaccines include SUISHOT® PT-100 (ChoongAng Vaccine Laboratories, South Korea) a combination killed PEDV and TGEV vaccine, and SUISHOT® PED a killed PEDV vaccine. The strain and subtypes offered through ChoonAng Vaccine Laboratories are unknown. Also Komipharm International Co., another South Korean company, offers a series of killed, live, and combination vaccines marketed under the tradename PRO-VAC® which include the PEDV strain SM98P of genogroup G1a. Qilu Animal Health Products Factory of China, also markets a combination killed vaccine in China containing PEDV and TGEV whose strain and subtypes are unknown.

The newly emerging porcine deltacorona virus (PDCoV), also known as swine deltacorona virus SDCoV or SDCV and used interchangeably herein, is similar to porcine epidemic diarrhea virus (PEDV), and often implicated with the morbidity in neonatal animals and contributing to the global spread and impact of the disease on the swine industry. PDCoV struck the US within a short time after PEDV with similar morbidity patterns. In the US, unlike PEDV, Deltacoronavirus does not seem to cause mortality in piglets but causes acute morbidity including severe diarrhea, vomiting and weight loss in piglets. Although milder than PEDV infection, PDCoV infection can result in 30-40% death rates in neonates. More severe disease may be seen in in co-infections with PEDV, rotaviruses and TGEV (Marthaler et al., 2014a; and Wang et al. 2014). Additionally, in May of 2015 a third new agent, porcine orthoreovirus (PORV) was detected in the US, potentially contributing to disease severity when present in co-infection. While more knowledge of the pathogenesis and clinical implication of PDCoV and other co-infective agents is needed, this recent co-emergence of PEDV and PDCoV has potentially enhanced the severity and impact of disease on herds.

Porcine Deltacoronavirus (PDCoV) is a member of the Coronaviridae family, genus Deltacoronavirus. The virus was first identified in Hong Kong SAR among porcine rectal swab samples collected in 2009 (Woo, P. C., et al., Discovery of seven novel Mammalian and avian coronaviruses in the genus Deltacoronavirus supports bat coronaviruses as the gene source of Alphacoronavirus and Betacoronavirus and avian coronaviruses as the gene source of Gammacoronavirus and Deltacoronavirus. J Virol, 2012. 86(7): p. 3995-4008) but was not known to be associated with any disease. SDCV, referred to herein as PDCoV, was first detected in US swine herds in February 2014 in Ohio and Indiana and has quickly spread to other states (Hu, H., et al., Isolation and Characterization of Porcine Deltacoronavirus from Pigs with Diarrhea in the United States. J Clin Microbiol, 2015. 53(5): p. 1537-1548; Chen, Q., et al., Pathogenicity and pathogenesis of a United States porcine Deltacoronavirus cell culture isolate in 5-day-old neonatal piglets. Virology, 2015. 482: p. 51-59; Li, G., et al., Full-Length Genome Sequence of Porcine Deltacoronavirus Strain USA/IA/2014/8734. Genome Announc, 2014. 2(2); Marthaler, D., et al., Rapid detection, complete genome sequencing, and phylogenetic analysis of porcine Deltacoronavirus. Emerg Infect Dis, 2014a. 20(8): p. 1347-50; Marthaler, D., et al., Complete Genome Sequence of Strain SDCV/USA/Illinois121/2014, a Porcine Deltacoronavirus from the United States. Genome Announc, 2014b. 2 (2); Wang, L., B. Byrum, and Y. Zhang, Detection and genetic characterization of Deltacoronavirus in pigs, Ohio, USA, 2014. Emerg Infect Dis, 2014. 20(7): p. 1227-30; Jung, K., et al., Pathogenicity of 2 porcine Deltacoronavirus strains in gnotobiotic pigs. Emerg Infect Dis, 2015. 21(4): p. 650-4). PDCoV was also detected in Korea in April 2014 (Lee, S. and C. Lee, Complete Genome Characterization of Korean Porcine Deltacoronavirus Strain KOR/KNU14-04/2014. Genome Announc, 2014. 2(6)), in mainland China (Dong, N., et al., Porcine Deltacoronavirus in Mainland China. Emerg Infect Dis, 2015. 21(12): p. 2254-5; and Song, D., et al., Newly Emerged Porcine Deltacoronavirus Associated With Diarrhoea in Swine in China: Identification, Prevalence and Full-Length Genome Sequence Analysis. Transbound Emerg Dis, 2015. 62(6): p. 575-80), and in Japan (ISERPD 2015).

Following identification, three research groups have confirmed that infection with cell culture derived material and/or tissue homogenate from clinical cases results in acute gastroenteric clinical signs in gnotobiotic and/or conventional piglets (Chen et al., 2015; Ma, Y., et al., Origin, evolution, and virulence of porcine Deltacoronaviruses in the United States. MBio, 2015. 6(2); and Vitosh-Sillman, S., et al. Histopathological and immunohistochemical characterization of pigs experimentally infected with porcine Deltacoronavirus. in American Association of Swine Veterinarians. 2015. Orlando, FL). PDCoV has been shown to cause acute diarrhea in sows and piglets in farrowing rooms 8-9 days post onset of clinical signs (Li et al. 2014), along with vomiting and dehydration (Ma, et al. 2015). The virus has similar clinical signs and symptoms as porcine enteric coronavirus (PEDV), transmissible gastroenteritis coronavirus (TGEV) and porcine orthoreoviruses (PORV).

A recent disclosure by Marx et al. (WO201600757A2) describes immunogenic compositions that allegedly protect swine from disease caused by porcine epidemic diarrhea virus (PEDV) and PDCoV. However, it is clear that the PEDV vaccine of Marx et al., based on the North American Colorado strain virus, is only partially protective (e.g., when comparing the modest reductions in the clinical symptoms of mortality, weight loss, or diarrhea) when challenged with an attenuated European INDEL-strain Calaf14 (causing only 23.8% mortality in control animals). Moreover, while Marx et al. also describe a deltacorona virus vaccine, protection or reduction of clinical symptoms of disease was not demonstrated with either a monovalent PDCoV vaccine or a bivalent PEDV and PDCoV vaccine

Therefore, what is needed is a PEDV and PDCoV vaccine capable of reducing the clinical signs of disease caused by PEDV and PDCoV and inducing protective immunity in immunized animals, including the reduction of viral shedding in immunized animals.

### SUMMARY OF THE INVENTION

The present invention provides immunogenic compositions, vaccines, and related methods that overcome deficiencies in the art. The present invention relates to immunogenic compositions which include inactivated/killed and/or recombinant forms of enveloped (+) single-stranded RNA viruses of the Coronaviridae family: porcine epidemic diarrhea virus, or PEDV and/or porcine Deltacoronavirus, or PDCoV. In particular, the application provides immunogenic compositions reducing the clinical symptoms in pigs with diseases associated with PEDV and PDCoV infection. The present PDCoV isolates (NSVL) (SEQ ID NO:1 and 2) and isolates PDCoV 2.0307 and PDCoV 5.0327 (SEQ ID NOs:5, 6, 9, and 10, respectively) have genetic profiles similar to those of other North American and Asian strains. The present PEDV isolate BI1251-125-10 (herein referred to as “125-10”) (SEQ ID NO:29, 32) is a virulent North American RNA virus strain with a genetic profile similar to those of other North American PEDVs reported of genogroup 2a. The PEDV derivative isolate “125-10 p30” (SEQ ID NO:34, 36). is a passage attenuate virus, also with a genetic profile consistent with the North American genogroup 2a.

Immunogenic compositions and vaccines of the invention comprise inactivated/killed PDCoV (e.g., chemically inactivated NSVL isolate (SEQ ID NO:1 and 2) and/or isolates PDCoV 2.0307 and PDCoV 5.0.327 (SEQ ID NO:5 and 6 and SEQ ID NO:9 and 10, respectively), and/or inactivated/killed PEDV (e.g., chemically inactivated PEDV isolate “125-10” or “125-10 p30” (SEQ ID NO:29 or 32 and SEQ ID NO:33 or 36, respectively)) and typically also includes an adjuvant. The vaccine may also include other components, such as preservative(s), antimicrobial agents, stabilizer(s), for example a stabilizer that can increase the shelf-life of the vaccine, emulsions, and antigens against other porcine pathogens.

Immunogenic compositions and vaccines of the invention comprise a Spike antigen, expressed in one non-limiting example in insect cells via a recombinant baculovirus expressing a PDCoV and/or PEDV Spike protein e.g., a PDCoV Spike protein (e.g., comprising nucleic acid sequences SEQ ID NO:3, 7, 11, 17 or 27 encoding the amino acid sequences SEQ ID NO:4, 8, 12, 18, or 28), and/or a PEDV Spike nucleic acid sequence (comprising SEQ ID NO:30, 34, 46, or 52) encoding amino acid sequence SEQ ID NO:31, 35, 47, or 53), and typically also includes an adjuvant. The vaccine may also include other components, such as preservative(s), stabilizer(s) and antigens against other porcine pathogens.

A preferred PDCoV and/or PEDV spike nucleic acid sequence suitable for use in the invention is a polynucleotide encoding a Spike polypeptide, said polynucleotide having at least at least will have at least at least 80%, 81%, 82%, 83%, 84%, 85%, 86%, 87%, 88%, or 89%, more preferably at least 90%, 91%, 92%, 93%, or 94%, and most preferably at least 95%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.41%, 97.42%, 97.43%, 97.44%, 97.45%, 97.46%, 97.47%, 97.48%, 97.49%, 97.5%, 97.51%, 97.52%, 97.53%, 97.54%, 97.55%, 97.56%, 97.57%, 97.58%, 97.59%, 97.6%, 97.61%, 97.62%, 97.63%, 97.64%, 97.65%, 97.66%, 97.67%, 97.68%, 97.69% 97.7%, 97.71%, 97.72%, 97.73%, 97.74%, 97.75%, 97.76%, 97.77%, 97.78%, 97.79%, 97.8%, 97.81%, 97.82%, 97.83%, 97.84%, 97.85%, 97.86%, 97.87%, 97.88%, 97.89%, 97.9%, 97.91%, 97.92%, 97.93%, 97.94%, 97.95%, 97.96%, 97.97%, 97.98%, 97.99%, 98%, 98.01%, 98.02%, 98.03%, 98.04%, 98.05%, 98.06%, 98.07%, 98.08%, 98.09%, 98.1%, 98.11%, 98.12%, 98.13%, 98.14%, 98.15%, 98.16%, 98.17%, 98.18%, 98.19%, 98.2%, 98.21%, 98.22%, 98.23%, 98.24%, 98.25%, 98.26%, 98.27%, 98.28%, 98.29%, 98.3%, 98.31%, 98.32%, 98.33%, 98.34%, 98.35%, 98.36%, 98.37%, 98.38%, 98.39%, 98.4%, 98.41%, 98.42%, 98.43%, 98.44%, 98.45%, 98.46%, 98.47%, 98.48%, 98.49%, 98.5%, 98.51%, 98.52%, 98.53%, 98.54%, 98.55%, 98.56%, 98.57%, 98.58%, 98.59%, 98.6%, 98.61%, 98.62%, 98.63%, 98.64%, 98.65%, 98.66%, 98.67%, 98.68%, 98.69%, 98.7%, 98.71%, 98.72%, 98.73%, 98.74%, 98.75%, 98.76%, 98.77%, 98.78%, 98.79%, 98.8%, 98.81%, 98.82%, 98.83%, 98.84%, 98.85%, 98.86%, 98.87%, 98.88%, 98.89%, 98.9%, 98.91%, 98.92%, 98.93%, 98.94%, 98.95%, 98.96%, 98.97%, 98.98%, 98.99%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%,99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83% 99.84%, 99.85%, 99.86%, 99.87%, 99.88%, 99.89%, 99.9%, 99.91%, 99.92%, 99.93%, 99.94%, 99.95%, 99.96%, 99.97%, 99.98% and 99.99% sequence identity to PDCoV Spike polypeptides SEQ ID NO:3, 7, 11, 17, or 27, or functional fragments thereof, and/or PEDV Spike SEQ ID NO:30, 34, 46, or 52, or functional fragments thereof. As used herein, it is in particular understood that the term “sequence identity to SEQ ID NO:X” or “identical SEQ ID NO:X”, respectively, is equivalent to the term “sequence identity with the sequence of SEQ ID NO:X over the length of SEQ ID NO:X” or “identical to the sequence of SEQ ID NO:X over the length of SEQ ID NO:X”, respectively.

A preferred spike polypeptide suitable for use in the invention is the polypeptide having the sequence set out in PDCoV Spike SEQ ID NOs:4, 8, 12, 18, or 28 and/or PEDV Spike SEQ ID NOs:31, 35, 47, or 53 having at least 80% homology with SEQ ID NOs:4, 8, 12, 18, 28, 31, 35, 47, and/or 53, for example at least 85% homology with SEQ ID NOs:4, 8, 12, 18, 28, 31, 35, 47, and/or 53, such as a least 85% homology with SEQ ID NOs:4, 8, 12, 18, 28, 31, 35, 47, and/or 53, such as at least 90% homology with SEQ ID NOs:4, 8, 12, 18, 28, 31, 35, 47, and/or 53, for example at least 95%, at least 98% or at least 99% homology with SEQ ID NOs:4, 8, 12, 18, 28, 31, 35, 47, and/or 53.

The terms “vaccine” and “immunogenic composition” are defined herein in a broad sense to refer to any type of biological agent in an administrable form capable of stimulating an immune response in an animal inoculated with the vaccine. Vaccines in general may be based on either the virus itself (e.g., killed/inactivated or attenuated) or an immunogenic (antigenic) component of the virus. In one embodiment of the invention, the vaccine (immunogenic composition) preferably includes the viral agent in a killed/inactivated form or an antigenic portion of the virus presented as a sub-unit vaccine. Herein, the term “reduction of clinical symptoms” when used in reference to an immunogenic composition refers to the lessening or amelioration (either partial or complete) of any of the symptoms associated with the disease or condition in question. Thus, reduction of clinical symptoms of pigs infected with PDCoV and/or PEDV by the present immunogenic compositions generally results in a diminishing of virus shedding and/or one or more of the clinical symptoms associated with infection with PDCoV and/or PEDV (e.g., acute watery diarrhea, acute vomiting, dehydration, anorexia, lethargy, depression, and high mortality in pigs less than 10 days old). Likewise, the reduction of clinical symptoms of pigs infected with PDCoV and/or PEDV by the present immunogenic compositions generally results in a diminishing of virus shedding and/or one or more of the clinical symptoms associated with infection with PDCoV and/or PEDV (e.g., acute watery diarrhea, acute vomiting, dehydration, anorexia, lethargy, depression, and high mortality in pigs less than 10 days old)

Those of skill in the art will understand that the compositions used herein may incorporate known injectable, physiologically acceptable sterile solutions. For preparing a ready-to-use solution for parenteral injection or infusion, aqueous isotonic solutions, e.g. saline or plasma protein solutions, are readily available. In addition, the immunogenic and vaccine compositions of the present invention can include veterinary-acceptable carriers, diluents, isotonic agents, stabilizers, or adjuvants.

Methods of the invention include, but are not limited to, a method of provoking an immune response against a PDCoV infection and/or PEDV infection, either singularly or in combination with each other or other co-infections, in a subject comprising the step of administering to the subject an immunogenic composition comprising an inactivated/killed PDCoV and/or PEDV, attenuated PDCoV and/or PEDV, and/or recombinant Spike antigens derived from PDCoV and/or PEDV. Preferably, the immune response is provoked against more than one serotype, or strain of PDCoV or PEDV. Compositions of the invention may be used to prevent a PDCoV infection, a PEDV infection, and/or a PEDV/PDCoV co-infection. Preferably, such immune response reduces the incidence of or severity of one or more clinical signs associated with or caused by the infection with one or more PDCoV genotypes and/or PEDV genotypes/genogroups.

Herein, suitable subjects and subjects in need to which compositions of the invention may be administered include animals in need of prophylactic treatment for a viral associated infection, disease, or condition. Animals in which the immune response is stimulated by use of compositions or methods of the invention include livestock, such as swine, bovines, poultry (e.g., chickens, ducks, geese, or turkeys) goats, and sheep, and domestic animals, such as mice, rabbits, dogs, cats, and horses. Preferred animals include porcine, murids, equids, lagomorphs, and bovids. Most preferably, an immune response is stimulated in pigs.

The invention also provides a method of reducing the incidence of or severity of one or more clinical signs associated with or caused by PDCoV infection, and/or PEDV infection comprising the step of administering an immunogenic composition of the invention that comprises inactivated/killed PDCoV vaccine, inactivated/killed PEDV vaccine, and/or in combination with a recombinant Spike antigen derived from PDCoV and/or PEDV as provided herewith and preferably a carrier molecule, such that the incidence of or the severity of a clinical sign of the PDCoV and/or PEDV infection is reduced by at least 10%, preferably at least 20%, even more preferred at least 30%, even more preferred at least 50%, even more preferred at least 70%, most preferred 100% relative to a subject that has not received the immunogenic composition as provided herewith. Such clinical signs include watery diarrhea, vomiting, and dehydration. Any of these clinical signs may result from an infection with PEDV having the genogroup of 2a or any other PEDV genogroup including G1a, G1b, or G2b. Likewise any of these clinical signs may result from an infection and/or a co-infection with PDCoV of varying genotypes.

In one embodiment, the present immunogenic compositions include a chemically inactivated form of PEDV. Vaccines which include chemically inactivated PDCoV (SEQ ID NOs:1 or 2, 5 or 6, 9 or 10) virus are particularly desirable. Additionally, the present immunogenic compositions may include a chemically inactivated form of PEDV. Vaccines which include chemically inactivated PEDV (SEQ ID NO:29 or 32 or SEQ ID NO:33 or 36) virus are particularly desirable. A variety of chemical inactivating agents known to those skilled in the art may be employed to inactivate the virus. Ethylenimine and related derivatives, such as binary ethylenimine (“BEI”) and acetylethylenimine, are examples of suitable chemical inactivating agents for use in inactivating the SDCo and/or PED virus. Other chemical inactivating agents, e.g., beta-propiolactone or aldehydes (such as formaldehyde and glutaraldehyde), can also be used to inactivate the virus.

The present immunogenic compositions and/or vaccines generally include an adjuvant which desirably may have bioadhesive properties, particularly where the virus is designed to be capable of intranasal administration. Examples of suitable adjuvants include cross-linked olefinically unsaturated carboxylic acid polymers, such as cross-linked acrylic acid polymers. As used herein the term “cross-linked acrylic acid polymer” refers to polymer and copolymers formed from a monomer mixture which includes acrylic acid as the predominant monomer in the mixture. Examples of suitable cross-linked acrylic acid polymers include those commercially available under the tradenames CARBOPOL® 934P and CARBOPOL® 971 (available from B. F. Goodrich Co., Cleveland, Ohio). One particularly suitable adjuvant for use in the present vaccines is a cross-linked acrylic acid polymer having a Brookfield viscosity of no more than about 20,000 cPs (as measured at 20 rpm as a 1.0 wt. % aqueous solution at pH 7.5). Where a bioadhesive adjuvant is desired, it may be advantageous to utilize an adjuvant which has a bioadhesive property of at least about 50 dynes/cm2 as measured between two pieces of freshly excised rabbit stomach tissue (as determined by the procedure described in U.S. Pat. No. 4,615,697).

The present invention also relates to a method of immunizing a subject, comprising administering to a subject any of the immunogenic compositions as described herein.

The term “immunizing” relates to an active immunization by the administration of an immunogenic composition to a subject to be immunized, thereby causing an immunological response against the antigen included in such immunogenic composition.

Preferably, immunization results in lessening of the incidence of the particular PDCoV infection in a herd, alone or in combination with PEDV infection, or in the reduction in the severity of clinical signs caused by or associated with the particular PDCoV and/or PEDV infection.

Further, the immunization of a subject in need with the immunogenic compositions as provided herewith, results in preventing infection of a subject by PDCoV and/or PEDV infection. Even more preferably, immunization results in an effective, long-lasting, immunological-response against PDCoV and/or PEDV infection. It will be understood that the said period of time will last more than 2 months, preferably more than 3 months, more preferably more than 4 months, more preferably more than 5 months, more preferably more than 6 months. It is to be understood that immunization may not be effective in all subjects immunized.

Preferably, a herd of subjects is envisaged in this context which normally, i.e. without immunization, would develop clinical signs normally caused by or associated with a PDCoV infection and/or PEDV infection. Whether the subjects of a herd are effectively immunized can be determined without further ado by the person skilled in the art. Preferably, the immunization shall be effective if clinical signs in at least 20%, at least 30%, at least 40%, at least 50%, at least 60%, at least 70%, at least 80%, at least 90%, still more preferably in at least 95% and most preferably in 100% of the subjects of a given herd are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, still more preferably by at least 95% and most preferably by 100% in comparison to subjects that are either not immunized or immunized with an immunogenic composition that was available prior to the present invention but subsequently infected by the particular PDCoV and/or PEDV.

Methods for preventing clinical signs caused by PDCoV and/or PEDV in a subject in need, or methods of protecting pigs against diseases associated with PDCoV and/or PEDV include administering an immunogenic composition and/or vaccine containing inactivated/killed PDCoV, and/or inactivated/killed PEDV, and/or Spike antigens derived from PDCoV and/or PEDV to the pigs. The vaccine can be administered using a variety of methods including intranasal, oral and/or parenteral (e.g., intramuscular) administration. In one embodiment of the method, for example, the inactivated PDCoV and/or the inactivated PEDV containing immunogenic composition is administered intramuscularly one or more times (e.g., at intervals of 2-4 weeks). In another embodiment of the method, for example, the inactivated PDCoV and/or inactivated PEDV containing vaccine is administered orally one or more times (e.g., at intervals of 2-4 weeks). In an alternative embodiment oral administration can be followed by and/or precede administration of the vaccine at least once, intramuscularly (e.g., 2-4 weeks after and/or before the parenteral administration of vaccine). Ideally, all pigs in a given herd are vaccinated at the prescribed intervals in order to protect against the spread of symptoms of the disease.

A method of producing an inactivated/killed PDCoV vaccine, an inactivated/killed PEDV vaccine, or a combination PDCoV/PEDV immunogenic composition is also provided. For the production of the PDCoV, the method typically includes inoculating swine testes cells with PDCo virus, e.g., with PDCo virus SEQ ID NO:1 or 2, 5 or 6, and 9 or 10. The inoculated swine testes cells are incubated, generally at least until CPE is, and then the PED virus is harvested from the incubated cells. The harvested virus-containing fluids can be treated with a chemical inactivating agent, such as binary ethylenimine, to form inactivated/killed PDCo virus. Typically, the inactivated virus is further processed, e.g., by concentration and blending with other components, to produce a commercial formulation. For example, the fluids containing the inactivated virus may be concentrated and blended with an adjuvant and/or antigen(s) to one or more other porcine pathogens. For the production of the PEDV, the method typically includes inoculating simian cells with PED virus, e.g., with PED virus SEQ ID NO:29 or SEQ ID NO:33. The inoculated simian cells are incubated, generally at least until CPE is observed, and then the PED virus is harvested from the incubated cells. The harvested virus-containing fluids can be treated with a chemical inactivating agent, such as binary ethylenimine, to form inactivated/killed PED virus. Typically, the inactivated virus is further processed, e.g., by concentration and blending with other components, to produce a commercial formulation. For example, the fluids containing the inactivated virus may be concentrated and blended with an adjuvant and/or antigen(s) to one or more other porcine pathogens.

A method of producing recombinantly expressed Spike antigen vaccines generated in insect cells via a recombinant baculovirus expressing PDCoV and/or PEDV Spike proteins is also provided. The method in one exemplary embodiment includes cloning the PDCoV Spike coding sequences (e.g., SEQ ID NOs:3, 7, 11, 17, or 27) and or/or PEDV Spike coding sequences (SEQ ID NO:30, 34, 46, or 52), including for example the PDCoV Spike-Fc antigen construct (SEQ ID NO:17), and/or the PDCoV Spike Baculodisplay construct (SEQ ID NO:27), and/or the PEDV 2a Spike construct (SEQ ID NO:46), and/or the PEDV 2b SPIKE construct (SEQ ID NO:52), into a baculovirus vector, and co-transfecting Sf9 insect cells.

The present application is also directed to a kit which includes in combination, (1) a dispenser capable of administering a vaccine to a pig; and (2) a chemically inactivated PDCoV, and/or a chemically inactivated PEDV, and/or recombinant Spike antigens derived from PDCoV and/or PEDV containing vaccine capable of protecting against diseases associated with PDCoV and/or PEDV. The kit may include a dispenser which is capable of dispensing its contents as droplets, e.g., as aerosol, atomized spray and/or liquid droplets, and a form of the vaccine which is capable of protecting against diseases associated with PDCoV and/or PEDV, for example when administered intranasally and/or intramuscularly.

Throughout this application, the text refers to various embodiments of the present compositions and/or related methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Complete genome nucleotide neighbor-joining tree for PDCoV isolates/strains. Scale indicates p-distance. GenBank accession numbers listed along with two letter state or three letter country code (CHN—China, TJK—Thailand, LAO—Laos).

FIG. 2 Spike amino acid neighbor-joining tree for PDCoV isolates/strains. Scale indicates p-distance. GenBank accession numbers listed along with two letter state or three letter country code (CHN—China, TJK—Thailand, LAO—Laos).

FIG. 3A Schematic diagram of PDCoV S1-IgG2a Fc Baculovirus-Protein Map of PDCoV S1-IgG2a Fc.

FIG. 3B Schematic diagram of PDCoVS BD Baculodisplay Baculovirus-Protein Map of PDCoVS BD.

FIG. 4 Serology Study with PDCoV Prototypes (Groups 1, 2, and 4, BaculoS-BD-PDCoV, Whole-cell PDCoV, and BaculoS-FC-PDCoV, respectively) compared to the placebo (Group 3).

FIG. 5 Serological response following vaccination with PDCoV Prototypes as detected by S1-IgG2a-Fc based ELISA: (Groups 1, 2, and 4, BaculoS-BD-PDCoV, Whole-cell PDCoV, and BaculoS-FC-PDCoV, respectively) compared to the placebo (Group 3).

FIG. 6A Schematic diagram of PEDV 2a BD Baculodisplay Baculovirus Protein map.

FIG. 6B Schematic diagram of PEDV 2b BD Baculodisplay Baculovirus-Protein map.

FIG. 7 Survivability by group and day after PEDV BD challenge.

FIG. 8 IgA levels in vaccinated animals.

### DETAILED DESCRIPTION

The invention provides immunogenic compositions including inactivated/killed, forms of PDCoV and/or PEDV and/or recombinantly expressed PDCoV and/or PEDV-Spike antigens. The immunogenic compositions are designed for protecting swine against diseases associated with PDCoV and/or PEDV. The immunogenic compositions typically include a chemically inactivated form of PDCoV and/or PEDV and those which include chemically inactivated/killed PDCoV and/or PEDV virus are particularly desirable. In another embodiment the immunogenic compositions include recombinant expressed PDCoV and/or PEDV Spike antigens generated, for example, in insect cells via a recombinant baculovirus expressing a PDCo V and/or PEDV Spike proteins.

Generally, the present invention provides an immunogenic composition comprising one or more antigens of porcine Deltacoronavirus (PDCoV) and an adjuvant, wherein in the porcine Deltacoronavirus (PDCoV) is any PDCoV: a.) that is encoded by SEQ ID NO:1, 5 or 9 and/or comprises the sequence of SEQ ID NO:1, 5 or 9 and/or comprises the RNA equivalent of SEQ ID NO:1, 5 or 9; b.) which sequence is at least 99% identical with SEQ ID NO:1, 5, or 9 and/or is at least 99% identical with the RNA equivalent of SEQ ID. NO:1; 5, or 9 c.) which spike protein is encoded by nucleic acid sequences of SEQ ID NO:3, 7, or 11; d.) which spike protein is encoded by a nucleic acid sequence that is at least 90% identical with the SEQ ID NO:3, 7, or 11; e.) that is encoded by SEQ ID NO:2, 6, or 10; or f.) which sequence is at least 99% identical SEQ ID NO:2, 6, or 10.

In a specific aspect the immunogenic composition according the sentence above, wherein the adjuvant is an oil-in-water emulsion.

In a further specific aspect of the invention, the immunogenic composition is as described in any of the above sentences, wherein the immunogenic composition is a recombinant antigen or an inactivated whole porcine Deltacoronavirus PDCoV antigen.

In a further specific aspect of the invention, the immunogenic composition is as described in any of the above sentences, wherein the antigen is an inactivated whole PDCoV antigen.

In a further specific aspect of the invention, the immunogenic composition is as described in any of the above sentences, wherein the PDCoV antigen is chemically inactivated.

In a further specific aspect of the invention, the immunogenic composition is as described in any of the above sentences, wherein the PDCoV antigen is chemically inactivated by treatment with a chemical inactivating agent which includes a compound selected from the group consisting of ethylenimine, binary ethylenimine, acetylethylenimine and mixtures thereof.

In a further specific aspect of the invention, the immunogenic composition is as described in any of the above sentences, wherein the PDCoV is chemically inactivated by treatment with binary ethylenimine.

In a further specific aspect of the invention, the immunogenic composition is as described in any of the above sentences, wherein the adjuvant is an EMULSIGEN® oil-in-water emulsion-based adjuvant.

In a further specific aspect of the invention, the immunogenic composition is as described in any of the above sentences, wherein an inactivated porcine Deltacoronavirus (PDCoV) comprises SEQ ID NO:1, 5, or 9 and/or comprises the RNA equivalent of SEQ ID NO:1, 5, or 9.

In a further specific aspect of the invention, the immunogenic composition is as described in any of the above sentences, wherein the PDCoV antigen is a recombinant antigen.

In a further specific aspect of the invention, the immunogenic composition is as described in any of the above sentences, wherein the recombinant antigen comprises one or more immunogenic components selected from the group consisting of: a.) an isolated nucleic acid encoding an antigen of porcine Deltacoronavirus (PDCoV) spike protein, wherein the recombinant Spike polypeptide has at least 90% homology with SEQ ID NO:4, 8, 12, 18, or 28; b.) a recombinant vector comprising the isolated nucleic acid of a); c.) the recombinant PDCoV Spike protein encoded by the nucleic acid of a); and d.) any combination thereof.

In a further specific aspect of the invention, the immunogenic composition is as described in any of the above sentences, wherein such immunogenic composition comprises a pharmaceutical acceptable carrier and/or an excipient.

In a further specific aspect of the invention, the immunogenic composition is as described in any of the above sentences, wherein the oil-in-water emulsion is an EMULSIGEN® oil-in-water emulsion-based adjuvant.

In a further specific aspect of the invention, the immunogenic composition is as described in any of the above sentences, wherein the immunogenic composition further comprises one or more additional antigens.

In a further specific aspect of the invention, the immunogenic composition is as described in any of the above sentences, wherein an additional antigen is an antigen of porcine epidemic diarrhea virus (PEDV), wherein the porcine epidemic diarrhea virus (PEDV) is any PEDV: a.) that is encoded by SEQ ID NO:29 or 33, and/or comprises the sequence of SEQ ID NO:29 or 33 and/or comprises the RNA equivalent of SEQ ID NO:29 or 33; b.) which sequence is at least 99% identical with SEQ ID NO:29 or 33 and/or is at least 99% identical with the RNA equivalent of SEQ ID. NO:29 or 33; c.) which spike protein is encoded by the nucleic acid sequence of SEQ ID NO:30, 34, 46, or 52; d.) which spike protein is encoded by a nucleic acid sequence that is at least 90% identical with SEQ ID NO:30, 34, 46, or 52; e.) that is encoded by SEQ ID NO:32 or 36; or f.) that is encoded by a sequence that is at least 99% identical to SEQ ID NO:32 or 36.

In a further specific aspect of the invention, the immunogenic composition is as described in any of the above sentences, wherein the additional PEDV antigen is a recombinant antigen or an inactivated whole porcine epidemic diarrhea virus (PEDV).

In a further specific aspect of the invention, the immunogenic composition is as described in any of the above sentences, wherein the antigen is an inactivated whole porcine epidemic diarrhea virus (PEDV).

In a further specific aspect of the invention, the immunogenic composition is as described in any of the above sentences, wherein the antigen is a recombinant PEDV antigen.

In a further specific aspect of the invention, the immunogenic composition is as described in any of the above sentences, wherein the recombinant antigen comprises one or more immunogenic components selected from the group consisting of: a.) an isolated nucleic acid encoding an antigen of porcine epidemic diarrhea virus (PEDV) spike protein, wherein the antigen has at least 90% homology with SEQ ID NO:31, 35, 47, or 53; b.) a recombinant vector comprising the isolated nucleic acid of a); c.) the recombinant PEDV Spike protein encoded by the nucleic acid of a); and d.) any combination thereof.

In a further specific aspect of the invention, the immunogenic composition is as described in any of the above sentences, wherein the recombinant antigen comprises one or more immunogenic components selected from the group consisting of: a structural protein M, E, or N of a porcine epidemic diarrhea virus (PEDV).

In a further specific aspect of the invention, the immunogenic composition is as described in any of the above sentences, wherein an immunogenic component is the isolated nucleic acid.

In a further specific aspect of the invention, the immunogenic composition is as described in any of the above sentences, wherein an immunogenic component is the recombinant vector.

In a further specific aspect of the invention, the immunogenic composition is as described in any of the above sentences, wherein an immunogenic component is the recombinant porcine epidemic diarrhea virus (PEDV) Spike protein.

In a further specific aspect of the invention, the immunogenic composition is as described in any of the above sentences, wherein an immunogenic component is a combination.

The present invention further concerns a method for reducing the clinical symptoms of disease associated with porcine Deltacoronavirus (PDCoV), comprising administering to a pig the immunogenic composition the immunogenic composition as described in any of the above sentences.

In a further specific aspect of the invention, the method is as described in any of the above sentences, comprising administering to a pig the immunogenic composition comprising an inactivated whole PDCoV antigen.

In a further specific aspect of the invention, the method is as described in any of the above sentences, comprising administering to a pig the immunogenic composition comprising the PDCoV antigen as a recombinant antigen.

In a further specific aspect of the invention, the method is as described in any of the above sentences, wherein such administered immunogenic composition comprises one or more immunogenic components selected from the group consisting of: a.) that is encoded by SEQ ID NO:1, and/or comprises the sequence of SEQ ID NOs:1, 5, or 9 and/or comprises the RNA equivalent of SEQ ID NO:1, 5, or 9; b.) which sequence is at least 99% identical with the SEQ ID NO:1, 5, or 9, and/or is at least 99% identical with the RNA equivalent of SEQ ID. NO:1, 5 or 9; c.) which Spike protein is encoded by nucleic acid sequences of SEQ ID NO:3, 7, 11, 17, 27; d.) which Spike protein is encoded by a nucleic acid sequence that is at least 90% identical with the SEQ ID NO:3, 7, 11, 17 or 27; e.) that is encoded by SEQ ID NO:2, 6, or 10; and f.) which sequence is at least 99% identical SEQ ID NO:2, 6, or 10.

In a further specific aspect of the invention, the method is as described in any of the above sentences, wherein such administered immunogenic composition comprises one or more immunogenic components selected from the group consisting of: a.) that is encoded by or comprises the sequence of SEQ ID NO:1, 5, or 9; b.) which sequence is at least 99% identical with the SEQ ID NO:1, 5, or 9; d.) which spike protein is encoded by nucleic acid sequences of SEQ ID NO:3, 7, 11, 17, or 27; and e.) which spike protein is encoded by a nucleic acid sequence that is at least 90% identical with the SEQ ID NO:3, 7, 11, 17, or 27.

In a further specific aspect of the invention, the method is as described in any of the above sentences, wherein such administered immunogenic composition comprises a recombinant antigen comprising one or more immunogenic components selected from the group consisting of: a.) an isolated nucleic acid encoding an antigen of porcine epidemic diarrhea virus porcine Deltacoronavirus (PDCoV) spike protein, wherein the recombinant Spike polypeptide has at least 90% homology with SEQ ID NO:4, 8, 12, 18, or 28; b.) a recombinant vector comprising the isolated nucleic acid of a); c.) the recombinant porcine Deltacoronavirus (PDCoV) Spike protein encoded by the nucleic acid of a); and d.) any combination thereof.

The present invention further concerns a method for reducing the clinical symptoms of disease associated with porcine Deltacoronavirus (PDCoV) and porcine epidemic diarrhea virus (PEDV) comprising administering to a pig the immunogenic composition of any of the immunogenic compositions as described in any of the above sentences, preferably wherein the immunogenic composition comprising one or more antigens of PDCoV and one or more additional antigens, wherein the additional antigen is an antigen of porcine epidemic diarrhea virus (PEDV).

In a further specific aspect of the invention, the method is as described in the above sentence, comprising administering to such pig the immunogenic composition wherein the additional PEDV antigen is a recombinant antigen or an inactivated whole porcine epidemic diarrhea virus (PEDV).

In a further specific aspect of the invention, the method is as described in the above sentence, comprises administering to such pig the immunogenic composition as described in any of the above sentences.

The present invention further concerns a kit for inducing an immunogenic response in a pig against diseases associated with porcine Deltacoronavirus (PDCoV) comprising: a.) a dispenser capable of administering an immunogenic composition to a pig; and b.) the immunogenic composition as described in any of the above sentences

The present invention further concerns a kit for inducing an immunogenic response in a pig against diseases associated with porcine Deltacoronavirus (PDCoV) comprising: a.) a dispenser capable of administering an immunogenic to a pig; and b.) the immunogenic composition as described in any of the above sentences, more specifically wherein the antigen is an inactivated whole PDCoV antigen, even more specifically wherein the PDCoV antigen is chemically inactivated; more specifically wherein the PDCoV antigen is chemically inactivated by treatment with a chemical inactivating agent which includes a compound selected from the group consisting of ethylenimine, binary ethylenimine, acetylethylenimine and mixtures thereof; preferably wherein the PDCoV is chemically inactivated by treatment with binary ethylenimine. more specifically wherein the adjuvant is an EMULSIGEN® oil-in-water emulsion-based adjuvant; and even more preferably wherein the inactivated porcine Deltacoronavirus (PDCoV) comprises SEQ ID NO:1, 5, or 9, and/or comprises the RNA equivalent of SEQ ID NO:1, 5, or 9.

The present invention further concerns a kit for inducing an immunogenic response in a pig against diseases associated with porcine Deltacoronavirus (PDCoV) comprising: a.) a dispenser capable of administering an immunogenic composition to a pig; and b.) the immunogenic composition as described in any of the above sentences, more specifically wherein the PDCoV antigen is a recombinant antigen, even more specifically wherein the recombinant antigen comprises one or more immunogenic components selected from the group consisting of: a.) an isolated nucleic acid encoding an antigen of porcine Deltacoronavirus (PDCoV) spike protein, wherein the recombinant Spike polypeptide has at least 90% homology with SEQ ID NO:4, 8, 12, 18, or 28; b.) a recombinant vector comprising the isolated nucleic acid of a); c.) the recombinant PDCoV Spike protein encoded by the nucleic acid of a); and d.) any combination thereof, even more specifically wherein such immunogenic composition comprises a pharmaceutical acceptable carrier and/or an excipient; and even more specifically wherein the oil-in-water emulsion is an EMULSIGEN® oil-in-water emulsion-based adjuvant.

The present invention further concerns a kit for inducing an immunogenic response in a pig against diseases associated with porcine Deltacoronavirus (PDCoV) and porcine epidemic diarrhea virus (PEDV) comprising a.) a dispenser capable of administering an immunogenic composition to a pig; and b.) the immunogenic composition as described in any of the sentences above, specifically wherein the additional antigen is the porcine epidemic diarrhea virus (PEDV) and is any PEDV antigen as described in any of the sentences above.

The present invention further concerns a kit for inducing an immunogenic response in a pig against diseases associated with porcine Deltacoronavirus (PDCoV) and porcine epidemic diarrhea virus (PEDV) comprising: a.) a dispenser capable of administering an immunogenic composition to a pig; and b.) and the immunogenic composition as described in any of the sentences above, specifically wherein the additional antigen is the porcine epidemic diarrhea virus (PEDV) is any recombinant PEDV antigen or an any inactivated whole porcine epidemic diarrhea virus (PEDV) as described in any of the sentences above.

The present invention further concerns a kit for inducing an immunogenic response in a pig against diseases associated with porcine Deltacoronavirus (PDCoV) and porcine epidemic diarrhea virus (PEDV) comprising: a.) a dispenser capable of administering an immunogenic composition to a pig; and b.) the immunogenic composition as described in any of the sentences above, specifically wherein the additional antigen is any recombinant PEDV antigen as described in any of the sentences above.

The present invention further concerns a method of producing a porcine Deltacoronavirus (PDCoV) immunogenic composition as described in any of the sentences above, further comprising: a.) inoculating swine testes cells with the porcine Deltacoronavirus (PDCoV); b.) incubating the inoculated swine testes cells; c.) harvesting porcine Deltacoronavirus (PDCoV) from the incubated cells; and d.) treating the harvested cells with a chemical inactivating agent, preferably with a compound selected from the group consisting of ethylenimine, binary ethylenimine, acetylethylenimine or a mixture thereof to form inactivated porcine Deltacoronavirus (PDCoV) antigen.

In a further specific aspect of the invention, the method is as described in the above sentence, wherein the porcine Deltacoronavirus (PDCoV) comprises the additional features: a.) a sequence that is encoded by or comprises the sequence of SEQ ID NO:1, 5, or 9, b.) a sequence that is at least 99% identical with the SEQ ID NO:1, 5, or 9, c.) a spike protein that is encoded by nucleic acid sequences of SEQ ID NO:3, 7, 11, 17, or 27; d.) a spike protein that is encoded by a nucleic acid sequence that is at least 90% identical with the SEQ ID NO:3, 7, 11, 17, or 27.

In a further specific aspect of the invention, the method is as described in either of the above sentences, wherein the porcine Deltacoronavirus (PDCoV) comprises the additional features SEQ ID NO:1, 5, or 9 and/or the RNA equivalent of SEQ ID NO:1, 5, or 9.

In a further specific aspect of the invention, the method is as described in any of the above sentences, wherein the swine testes cells are AI-ST cells.

In a further specific aspect of the invention, the method is as described in any of the above sentences, wherein the chemical inactivating agent includes binary ethylenimine.

In a further specific aspect of the invention, the method is as described in any of the above sentences, further comprising the additional feature of adding an oil-in-water emulsion-based adjuvant EMULSIGEN® to the porcine Deltacoronavirus (PDCoV) antigen.

The present invention further concerns a method of producing an immunogenic composition comprising the recombinant porcine Deltacoronavirus (PDCoV) antigen as described in any of the preceding sentences comprising: a.) expressing an antigen of porcine Deltacoronavirus (PDCoV) in a host cell, b.) harvesting the antigen of porcine Deltacoronavirus (PDCoV) cells; and c.) adding an oil-in-water emulsion-based adjuvant to the porcine Deltacoronavirus (PDCoV) antigen of step b).

In a further specific aspect of the invention, the method is as described in the above sentence, wherein the porcine Deltacoronavirus (PDCoV) antigen comprises: a.) an isolated nucleic acid encoding an antigen of porcine Deltacoronavirus (PDCoV) spike protein, wherein the recombinant Spike polypeptide has at least 90% homology with SEQ ID NO: 3, 7, 11, 17, or 27; b.) a recombinant vector comprising the isolated nucleic acid of a); c.) the recombinant porcine Deltacoronavirus (PDCoV) Spike protein encoded by the nucleic acid of a); and d.) any combination thereof.

In a further specific aspect of the invention, the method is as described in any of the above sentences, wherein the antigen of porcine Deltacoronavirus (PDCoV) is expressed by a recombinant baculovirus vector.

In a further specific aspect of the invention, the method is as described in any of the above sentences, wherein the antigen of porcine Deltacoronavirus (PDCoV) is expressed in insect cells.

In a further specific aspect of the invention, the method is as described in any of the above sentences, wherein as an additional feature the oil-in-water based adjuvant is an EMULSIGEN® oil-in-water emulsion-based adjuvant.

The present invention further concerns the immunogenic composition comprising PDCoV as described in any of the above sentences, for the use for the reduction of symptoms of disease associated with porcine Deltacoronavirus (PDCoV).

The present invention further concerns the immunogenic composition comprising PDCoV and at least one additional antigen, wherein at least one additional antigen is an antigen of porcine epidemic diarrhea virus (PEDV) for the use for the reduction symptoms of disease associated with porcine Deltacoronavirus (PDCoV) and/or porcine epidemic virus (PEDV).

In a further specific aspect of the invention, the immunogenic composition is as described in any of the above sentences, further comprising one or more antigens of PEDV genotype 2a, specifically the PEDV is of North American origin.

In a further specific aspect of the invention, the immunogenic composition is as described in any of the above sentences further comprising one or more antigens of PDCoV wherein the PDCoV is of North American origin.

In more general terms, a variety of chemical inactivating agents known to those skilled in the art may be employed to inactivate the virus. Ethylenimine and related derivatives, such as binary ethylenimine (BEI) and acetylethylenimine, are examples of suitable chemical inactivating agents for use in inactivating the PED virus. Other chemical inactivating agents, e.g., beta-propiolactone, aldehydes (such as formaldehyde) and/or detergents (e.g., Tween® detergent, Triton® X, or alkyl trimethylammonium salts) can also be used to inactivate the virus. The inactivation can be performed using standard methods known to those of skill in the art. Samples can be taken at periodic time intervals and assayed for residual live virus. Monitoring of cytopathic effect on an appropriate cell line and/or fluorescent staining with an appropriate specific monoclonal or polyclonal antibody can be used to detect the presence of residual live virus.

Inactivation with BEI can be accomplished by combining a stock BEI solution (e.g., a solution formed by adding 0.1-0.2 M 2-bromo-ethylamine hydrobromide to 0.1-0.2 N aqueous NaOH) with viral fluids to a final concentration of about 1-5 mM BEI. Inactivation is commonly performed by holding the BEI-virus mixture at 35-40° C. (e.g., 37° C.) with constant mixing for 24-72 hours. Virus inactivation can be halted by the addition of sodium thiosulfate solution to a final concentration in excess of the BEI concentration (e.g., addition of sodium thiosulfate at 17% of the volume of BEI to neutralize excess BEI) followed by mixing.

The present immunogenic compositions usually include an adjuvant and, if desired, one or more emulsifiers such as Tween® detergent incorporated with the inactivated/killed PEDV. Suitable adjuvants include, for example, vitamin E acetate solubilisate, aluminum hydroxide, aluminum phosphate or aluminum oxide, (mineral) oil emulsions, non-ionic detergents, squalene and saponins. Other adjuvants which may be used include an oil based adjuvants such as Freund's complete adjuvant (FCA), and Freund's incomplete adjuvant (FIA). It has been found that cross-linked olefinically unsaturated carboxylic acid polymers, such as CARBOPOL® 971 polymer, are particularly suitable adjuvants for use in the present inactivated PEDV immunogenic compositions.

Examples for suitable oil-in water emulsions are EMULSIGEN® based adjuvants, such as EMULSIGEN® (an oil-in-water emulsion), EMULSIGEN-D® (an oil-in-water) with dimethyldioctadecylammonium bromide (DDA)), EMULSIGEN-PR (an oil-in-water) with a proprietary immunostimulant), EMULSIGEN-75® (a double adjuvant comprised of an oil-in-water) with a cross-linked polymer), and EMULSIGEN®-BCL (an oil-in-water emulsion that is free of animal origin components). (MVP Technologies, Inc. Omaha, Nebr., USA). Pharmaceutical/vaccine compositions that comprise inactivated PEDV or recombinant PEDV proteins, have been effectively adjuvanted with oil-in water emulsions, preferably with such EMULSIGEN®-based adjuvants, more preferably with EMULSIGEN® (an oil-in-water emulsion that is free of animal origin components) and EMULSIGEN®-BCL (an oil-in-water emulsion that is free of animal origin components).

It is generally advantageous to formulate the present compositions in dosage unit form for ease of administration and uniformity of dosage. Dosage unit form as used herein refers to physically discrete units suited as unitary dosages for the mammalian subjects to the treated; each unit containing a predetermined quantity of the active material calculated to produce the desired therapeutic effect in association with the required pharmaceutical carrier. The specification for the dosage unit forms of inactivated/killed PDCoV and/or PEDV, and/or recombinantly expressed PDCoV and/or PEDV antigens are dictated by and depend on among other factors (a) the unique characteristics of the active material and the particular therapeutic effect to be achieved; (b) the limitations inherent in the art of compounding such active material for the treatment of disease; and (c) the manner of intended administration of the dosage unit form.

The principal active ingredient is typically compounded for convenient and effective administration in effective amounts with a suitable pharmaceutically acceptable carrier in dosage unit form as disclosed herein. A unit dosage form can, for example, contain the PDCoV and/or PEDV antigen in amounts ranging from 1 to about 5 relative potency units (“RPUs”). This amount of the antigen is generally present in from about 1 to about 25/ml of carrier. In the case of compositions containing supplementary active ingredients, the dosages are determined by reference to the usual dose and manner of administration of the supplementary active ingredients.

The present vaccines typically include inactivated PDCoV and/or PEDV formulated with a pharmaceutically acceptable carrier. The pharmaceutical forms suitable for injectable use commonly include sterile aqueous solutions (where water soluble) or dispersions and sterile powders for the extemporaneous preparation of sterile injectable solutions or dispersion. The formulation should desirably be sterile and fluid to the extent that easy syringability exists. The dosage form should be stable under the conditions of manufacture and storage and typically is preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. The proper fluidity of the solution can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabenes, chlorobutanol, phenol, sorbic acid, thimerosal (sodium ethylmercuri-thiosalicylate), deomycin, gentamicin and the like. In many cases it may be preferable to include isotonic agents, for example, sugars or sodium chloride. Prolonged absorption of the injectable compositions, if desired, can be brought about by the use in the compositions of agents delaying absorption, for example, aluminum monostearate and gelatin.

Sterile injectable solutions may be prepared by incorporating the inactivated virus in the desired amount in an appropriate solvent with various other ingredients as enumerated above, as required, followed by filtered sterilization. Generally, dispersions can be prepared by incorporating the various active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from a previously sterile-filtered solution thereof.

It may also be advantageous to add a stabilizer to the present compositions to improve the stability of inactivated virus. Suitable stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin). If desired, the formulation may be buffered by methods known in the art, using reagents such as alkali metal phosphates, e.g., sodium hydrogen phosphate, sodium dihydrogen phosphate, potassium hydrogen phosphate and/or potassium dihydrogen phosphate. Other solvents, such as ethanol or propylene glycol, can be used to increase solubility of ingredients in the vaccine formulation and/or the stability of the solution. Further additives which can be used in the present formulation include conventional antioxidants and conventional chelating agents, such as ethylenediamine tetraacetic acid (EDTA).

The compositions and methods of the present invention may be illustrated by the following examples, which are presented to illustrate the present invention and to assist in teaching one of ordinary skill how to make and use the same. These examples are not intended in any way to narrow or otherwise limit the scope of the present invention.

The practice of the present invention will employ, unless otherwise indicated, conventional techniques of molecular biology, microbiology, recombinant DNA technology, protein chemistry and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); Protein purification methods—a practical approach (E. L. V. Harris and S. Angal, eds., IRL Press at Oxford University Press); and Handbook of Experimental Immunology, Vols. I-IV (D. M. Weir and C. C. Blackwell eds., 1986, Blackwell Scientific Publications).

It is to be understood that this invention is not limited to particular DNA, RNA, polypeptide sequences, or process parameters as such may, of course, vary. It is also to be understood that the terminology used herein is for the purpose of describing particular embodiments of the invention only, and is not intended to be limiting. It must be noted that, as used in this specification and the appended claims, the singular forms “a”, “an” and “the” include plural referents unless the content clearly dictates otherwise. Thus, for example, reference to “an antigen” includes a mixture of two or more antigens; reference to “an excipient” includes mixtures of two or more excipients, and the like.

### Definitions

Unless defined otherwise, all technical and scientific terms used herein have the same meaning as is commonly understood by one of skill in the art to which this invention belongs at the time of filing. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. All patents and publications referred to herein are incorporated by reference herein.

“Protection against disease”, “protective immunity”, “functional immunity” and similar phrases, means a response against a disease or condition generated by administration of one or more therapeutic compositions of the invention, or a combination thereof, that results in fewer deleterious effects than would be expected in a non-immunized subject that has been exposed to disease or infection. That is, the severity of the deleterious effects of the infection are lessened in a vaccinated subject. Infection may be reduced, slowed, or possibly fully prevented, in a vaccinated subject. Herein, where complete prevention of infection is meant, it is specifically stated. If complete prevention is not stated then the term includes partial prevention.

Herein, “reduction of the incidence and/or severity of clinical signs” or “reduction of clinical symptoms” means, but is not limited to, reducing the number of infected subjects in a group, reducing or eliminating the number of subjects exhibiting clinical signs of infection, or reducing the severity of any clinical signs that are present in one or more subjects, in comparison to wild-type infection. For example, it should refer to any reduction of pathogen load, pathogen shedding, reduction in pathogen transmission, or reduction of any clinical sign symptomatic of PEDV. Preferably these clinical signs are reduced in one or more subjects receiving the therapeutic composition of the present invention by at least 10% in comparison to subjects not receiving the composition and that become infected. More preferably clinical signs are reduced in subjects receiving a composition of the present invention by at least 20%, preferably by at least 30%, more preferably by at least 40%, and even more preferably by at least 50%.

The term “increased protection” herein means, but is not limited to, a statistically significant reduction of one or more clinical symptoms which are associated with infection by an infectious agent, preferably PDCoV and/or PEDV, in a vaccinated group of subjects vs. a non-vaccinated control group of subjects. The term “statistically significant reduction of clinical symptoms” means, but is not limited to, the frequency in the incidence of at least one clinical symptom in the vaccinated group of subjects is at least 10%, preferably 20%, more preferably 30%, even more preferably 50%, and even more preferably 70% lower than in the non-vaccinated control group after the challenge with the infectious agent.

“Long-lasting protection” shall refer to “improved efficacy” that persists for at least 3 weeks, but more preferably at least 3 months, still more preferably at least 6 months. In the case of livestock, it is most preferred that the long lasting protection shall persist until the average age at which animals are marketed for meat.

An “immunogenic or immunological composition” refers to a composition of matter that comprises at least one porcine deltacorona virus and/or porcine epidemic diarrhea virus and/or immunogenic portions thereof that elicit an immunological response in the host of a cellular or antibody-mediated immune response to the composition. In a preferred embodiment of the present invention, an immunogenic composition induces an immune response and, more preferably, confers protective immunity against one or more of the clinical signs of PDCoV and/or PEDV infection.

The immunogenic composition as used herein also refers to a composition that comprises any of the PDCoV and/or PEDV Spike polypeptides described herein. According to a further embodiment, such immunogenic composition further comprises at least a portion of a viral vector expressing said PDCoV and/or PEDV Spike protein, preferably of a recombinant baculovirus. Moreover, the immunogenic composition can comprise i) any of the PDCoV or PEDV proteins described above, preferably in concentrations described above, ii) at least a portion of the viral vector expressing said PDCoV and/or PEDV Spike protein, preferably of a recombinant baculovirus, and iii) a portion of the cell culture supernatant.

Thus according to one aspect, the present invention relates to a method for reducing the percentage of PDCoV and/or PEDV infections in a herd of pigs comprising the step administering to said pig(s) an effective amount of PDCoV and/or PEDV Spike antigen or an immunogenic composition comprising PDCoV and/or PEDV antigen, wherein the PDCoV and/or PEDV antigen is recombinant PDCoV and/or PEDV Spike antigen, preferably a baculovirus expressed PDCoV and/or PEDV Spike protein. Preferably those recombinant or baculovirus expressed PDCoV and/or PEDV Spike having the sequence as described herein.

An “immune response” or “immunological response” means, but is not limited to, the development of a cellular and/or antibody-mediated immune response to the composition or vaccine of interest. Usually, an immune or immunological response includes, but is not limited to, one or more of the following effects: the production or activation of antibodies, B cells, helper T cells, suppressor T cells, and/or cytotoxic T cells, directed specifically to an antigen or antigens included in the composition or vaccine of interest. Preferably, the host will display either a therapeutic or a protective immunological (memory) response such that resistance to new infection will be enhanced and/or the clinical severity of the disease reduced. Such protection will be demonstrated by either a reduction in number of symptoms, severity of symptoms, or the lack of one or more of the symptoms associated with the infection of the pathogen, a delay in the of onset of viremia, reduced viral persistence, a reduction in the overall viral load and/or a reduction of viral excretion.

“Immunologically protective amount” or “immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity. “Immunogenic” means evoking an immune or antigenic response. Thus an immunogenic composition would be any composition that induces an immune response.

“Therapeutically effective amount” refers to an amount of an antigen or vaccine that would induce an immune response in a subject receiving the antigen or vaccine which is adequate in preventing or reducing the clinical signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

As used herein, “a pharmaceutical-or veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In some preferred embodiments, and especially those that include lyophilized immunogenic compositions, stabilizing agents for use in the present invention include stabilizers for lyophilization or freeze-drying.

In some embodiments, the immunogenic composition of the present invention contains an adjuvant. “Adjuvants” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion.

The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al. The Theory and Practical Application of Adjuvants (Ed.Stewart-Tull, D. E. S.), John Wiley and Sons, NY, pp51-94 (1995) and Todd et al. Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

Examples for suitable oil-in water emulsions are EMULSIGEN® based adjuvants, such as EMULSIGEN® (an oil-in-water emulsion o/w), EMULSIGEN-D® (an oil-in-water (o/w) with dimethyldioctadecylammonium bromide (DDA)), EMULSIGEN-P® (an oil-in-water (o/w) with a proprietary immunostimulant), EMULSIGEN-75® (a double adjuvant comprised of an oil-in-water (o/w) with a cross-linked polymer), and EMULSIGEN®-BCL (an oil-in-water emulsion that is free of animal origin components). (MVP Laboratories, Inc. Omaha, Nebr., USA). Pharmaceutical/vaccine compositions that comprise inactivated PDCoV, inactivated PEDV, and/or recombinant PDCoV and/or PEDV proteins, have been effectively adjuvanted with oil-in water emulsions, preferably with such EMULSIGEN®-based adjuvants, more preferably with EMULSIGEN® (an oil-in-water emulsion o/w) and/or EMULSIGEN®-BCL (an oil-in-water emulsion that is free of animal origin components).

Examples of suitable adsorbent aluminum hydroxide gels for use in veterinary vaccines include REHYDRAGEL®, REHYDRAGEL-CG®; REHYDRAGEL-LV; REHYDRAGEL-HPA; REHYDRAPHOS (General Chemical, Berkeley Heights, New Jersey, USA).

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL® (also known as polyacrylic acid); (BF Goodrich, Ohio, USA) are particularly appropriate. They are cross-linked with an allyl sucrose or with allyl pentaerythritol. Among then, there may be mentioned CARBOPOL® 974P (also known as polyacrylic acid), CARBOPOL® 934P (also known as polyacrylic acid) and CARBOPOL® 971P (also known as polyacrylic acid). Most preferred is the use of CARBOPOL® 971P (also known as polyacrylic acid). Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, or muramyl dipeptide among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

“Safety” refers to the absence of adverse consequences in a vaccinated animal following vaccination, including but not limited to: potential reversion of a viral-based vaccine to virulence, clinically significant side effects such as persistent, systemic illness or unacceptable inflammation at the site of vaccine administration.

The terms “vaccination” or “vaccinating” or variants thereof, as used herein means, but is not limited to, a process which includes the administration of an immunogenic composition of the invention that, when administered to an animal, elicits, or is able to elicit—directly or indirectly—, an immune response in the animal against PDCoV and/or PEDV.

“Mortality”, in the context of the present invention, refers to death caused by PDCoV infection, PEDV infection, and/or co-infection with PDCoV and PEDV, and includes the situation where the infection is so severe that an animal is euthanized to prevent suffering and provide a humane ending to its life.

Herein, “effective dose” means, but is not limited to, an amount of antigen that elicits, or is able to elicit, an immune response that yields a reduction of clinical symptoms in an animal to which the antigen is administered.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073(1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al. Nucleic Acids Research, 12(1):387 (1984)), BLASTP, BLASTN and FASTA (Altschul, S. F. et al. J. Molec. Biol., 215:403-410 (1990). The BLAST programs are publicly available from NCBI and other sources (BLAST Manual, Altschul, S. et al. NCVI NLM NIH Bethesda, MD 20894, Altschul, S. F. et al. J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence homology.

A “conservative substitution” refers to the substitution of an amino acid residue or with another amino acid residue having similar characteristics or properties including size, hydrophobicity, etc., such that the overall functionality does not change significantly.

“Sequence homology”, as used herein, refers to a method of determining the relatedness of two sequences discounting conservative substitutions. To determine sequence homology, two or more sequences are optimally aligned, and gaps are introduced if necessary. In other words, to obtain a polypeptide having 95% sequence homology with a reference sequence, 85%, preferably 90%, even more preferably 95% of the amino acid residues in the reference sequence must match or comprise a conservative substitution with another amino acid, or a number of amino acids up to 15%, preferably up to 10%, even more preferably up to 5% of the total amino acid residues, not including conservative substitutions, in the reference sequence may be inserted into the reference sequence. Preferably the homolog sequence comprises at least a stretch of 50, even more preferred of 100, even more preferred of 250, even more preferred of 500 amino acids.

The terms “sequence identity” or “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length. When sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.”

A sequence comparison may be carried out over the entire lengths of the two sequences being compared or over fragment of the two sequences. Typically, the comparison will be carried out over the full length of the two sequences being compared. However, sequence identity may be carried out over a region of, for example, twenty, fifty, one hundred or more contiguous amino acid residues.

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6,or 4 and a length weight of 1, 2, 3, 4, 5, or 6. The skilled person will appreciate that all these different parameters will yield slightly different results but that the overall percentage identity of two sequences is not significantly altered when using different algorithms.

The protein sequences or nucleic acid sequences of the present invention can further be used as a “query sequence” to perform a search against public databases to, for example, to identify other family members or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

The claimed PEDV of the invention shall also encompass variants of the PEDV isolate 1251-125-10 (“125-10”) and variants of sub-fragments thereof. Such variants have essentially the same immunological properties as characteristic of the Oklahoma strain (SEQ ID NO 29 and 34). The term “having essentially the same immunological properties” encompass (but is not restricted to) that said variants are essentially effective in treating or preventing the clinical signs caused by PEDV as described below or in improving the efficacy parameters as described below.

The claimed PDCoV of the invention shall also encompass variants of the PDCoV isolates NSVL, PDCoV 2.0307, and PDCoV 5.0327 and variants of sub-fragments thereof. Such variants have essentially the same immunological properties as characteristic of the NSVL strain (SEQ ID NO: 1 and 2), and isolates PDCoV 2.0307 (SEQ ID NO:5 and 6) and PDCoV 5.0327 (SEQ ID NO:9 and 10). The term “having essentially the same immunological properties” encompass (but is not restricted to) that said variants are essentially effective in treating or preventing the clinical signs caused by PDCoV as described herein or in improving the efficacy parameters as described herein. Besides the various PDCoV strains that may be used in a vaccine, recombinant SPIKE protein antigens, including subfragments thereof, may also be used in a vaccine. Likewise, exemplary spike protein sequences include, but are not limited to, those with essentially the same immunological properties of the PDCoV isolates or ‘variants’ listed below.

The term “variant” with respect to PDCoV sequences (SEQ ID NO:1, 2, 3, 4, 5, 6, 7, 8, 9, 10, 11, 12,17, 18, 27 and 28) or PEDV sequences SEQ ID NO:29, 30, 31, 32, 33, 34, 35, 36, 46, 47, 52, and 53) (e.g., a polypeptide or nucleic acid sequence) is intended to mean substantially similar sequences. For nucleotide sequences comprising an open reading frame, variants include those sequences that, because of the degeneracy of the genetic code, encode the identical amino acid sequence of the native protein. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein for the purposes of codon optimization. Generally, nucleotide sequence variants of the invention will have at least at least 80%, 81%, 82%, 83%, 84%, 85%, 86%, 87%, 88%, or 89%, more preferably at least 90%, 91%, 92%, 93%, or 94%, and most preferably at least 95%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.41%, 97.42%, 97.43%, 97.44%, 97.45%, 97.46%, 97.47%, 97.48%, 97.49%, 97.5%, 97.51%, 97.52%, 97.53%, 97.54%, 97.55%, 97.56%, 97.57%, 97.58%, 97.59%, 97.6%, 97.61%, 97.62%, 97.63%, 97.64%, 97.65%, 97.66%, 97.67%, 97.68%, 97.69% 97.7%, 97.71%, 97.72%, 97.73%, 97.74%, 97.75%, 97.76%, 97.77%, 97.78%, 97.79%, 97.8%, 97.81%, 97.82%, 97.83%, 97.84%, 97.85%, 97.86%, 97.87%, 97.88%, 97.89%, 97.9%, 97.91%, 97.92%, 97.93%, 97.94%, 97.95%, 97.96%, 97.97%, 97.98%, 97.99%, 98%, 98.01%, 98.02%, 98.03%, 98.04%, 98.05%, 98.06%, 98.07%, 98.08%, 98.09%, 98.1%, 98.11%, 98.12%, 98.13%, 98.14%, 98.15%, 98.16%, 98.17%, 98.18%, 98.19%, 98.2%, 98.21%, 98.22%, 98.23%, 98.24%, 98.25%, 98.26%, 98.27%, 98.28%, 98.29%, 98.3%, 98.31%, 98.32%, 98.33%, 98.34%, 98.35%, 98.36%, 98.37%, 98.38%, 98.39%, 98.4%, 98.41%, 98.42%, 98.43%, 98.44%, 98.45%, 98.46%, 98.47%, 98.48%, 98.49%, 98.5%, 98.51%, 98.52%, 98.53%, 98.54%, 98.55%, 98.56%, 98.57%, 98.58%, 98.59%, 98.6%, 98.61%, 98.62%, 98.63%, 98.64%, 98.65%, 98.66%, 98.67%, 98.68%, 98.69%, 98.7%, 98.71%, 98.72%, 98.73%, 98.74%, 98.75%, 98.76%, 98.77%, 98.78%, 98.79%, 98.8%, 98.81%, 98.82%, 98.83%, 98.84%, 98.85%, 98.86%, 98.87%, 98.88%, 98.89%, 98.9%, 98.91%, 98.92%, 98.93%, 98.94%, 98.95%, 98.96%, 98.97%, 98.98%, 98.99%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%,99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83% 99.84%, 99.85%, 99.86%, 99.87%, 99.88%, 99.89%, 99.9%, 99.91%, 99.92%, 99.93%, 99.94%, 99.95%, 99.96%, 99.97%, 99.98% and 99.99% sequence identity compared to a reference sequence using one of the alignment programs described using standard parameters.

The term “variant” with respect to PDCoV sequences (SEQ ID NO:1, 2, 3, 4, 5, 6, 7, 8, 9, 10, 11, 12,17, 18, 27 and 28) (e.g., a polypeptide or nucleic acid sequence) is intended to mean substantially similar sequences. Thus immunogenic compositions of the invention can effectively incorporate all recognized strains or isolates of PDCoV, including preferably, but not necessarily limited to, all strains that have at least about 90% overall nucleotide identity to isolate NSVL-SCDV (Porcine Deltacoronavirus strain USA/IL/2014/026PDV_P11) deposited as GenBank accession No. KP981395.1; isolate CHN-AH-2004, deposited as GenBank accession No. KP757890; isolate CHN-HB-2014, deposited as GenBank accession No. KP757891; isolate CHN-JS-2014, deposited as GenBank accession No.KP757892; isolate PDCoV/CHJXNI2/2015, deposited as GenBank accession No. KR131621; strain USA/Arkansas61/2015, deposited as GenBank accession No. KR150443; strain USA/Minnesota442/2014, deposited as GenBank accession No. KR265847; strain USA/Minnesota214/2014, deposited as GenBank accession No. KR265848; strain USA/Michigan447/2014, deposited as GenBank accession No. KR265849; strain USA/Michigan448/2014, deposited as GenBank accession No. KR265850; strain USA/Indiana453/2014, deposited as GenBank accession No. KR265851; strain USA/Illinois 449/2014, deposited as GenBank accession No. KR265852; strain USA/Minnesota/2013 deposited as GenBank accession No. KR265853; strain USA/Minnesota454/2014 deposited as GenBank accession No. KR265854; strain USA/Minnesota455/2014 deposited as GenBank accession No. KR265855; strain USA/Illinois272/2014 deposited as GenBank accession No. KR265856; strain USA/Illinois273/2014 deposited as GenBank accession No. KR265857; strain USA/NorthCarolina452/2014 deposited as GenBank accession No. KR265858; strain USA/Minnesota159/2014 deposited as GenBank accession No. KR265859; strain USA/Nebraska209/2014 deposited as GenBank accession No. KR265860; strain USA/Nebraska210/2014 deposited as GenBank accession No. KR265861; strain USA/Ohio444/2014 deposited as GenBank accession No. KR265862; strain USA/Ohio445/2014 deposited as GenBank accession No. KR265863; strain USA/Minnesota292/2014 deposited as GenBank accession No. KR265864; strain USA/Iowa459/2014 deposited as GenBank accession No. KR265865; strain CH/SXD1/2015 deposited as GenBank accession No. KT021234; strain CH/Sichuan/S27/2012 deposited as GenBank accession No. KT266822; isolate CHN-HN-2014 deposited as GenBank accession No. KT336560; strain PDCoV/Swine/Thailand/S5011/2015 deposited as GenBank accession No. KU051641; strain PDCoV/Swine/Thailand/S5015L/2015 deposited as GenBank accession No. KU051649; strain NH deposited as GenBank accession No. KU981059; strain NH isolate passage 0 deposited as GenBank accession No. KU981060; NH isolate passage 5 deposited as GenBank accession No. KU981061; strain NH isolate passage 10 deposited as GenBank accession No. KU981062; isolate TT_1115 deposited as GenBank accession No. KU984334; strain PDCoV/USA/Iowa136/2015 deposited as GenBank accession No. KX022602; strain PDCoV/USA/Minnesota140/2015 deposited as GenBank accession No. KX022603; strain PDCoV/USA/Nebraska137/2015 deposited as GenBank accession No. KX022604; strain PDCoV/USA/Nebraska145/2015 deposited as GenBank accession No. KX022605; and isolate P1_16_BTL_0115/PDCoV/2016/Lao deposited as GenBank accession No. KX118627.

The term “genogroup” as it is known in the art refers to related viruses within a genus; which may be further subdivided into genetic clusters. Identified genogroups of PEDV include group G1, comprising subgroups G1a, G1b, R (attenuated/adapted); and G2, comprising subgroups G2a, and G2b. Members of the G2a genogroup include the Chinese strain AH2012 (GenBank accession no: KC210145) and the North American strains, sharing several unique nucleotides changes. Strains MN and IA2 had 99.6% and strain IA1 had 99.5% nucleotide identity with AH2012, respectively. Researchers have speculated that an AH2012-like virus was possibly transmitted to the eastern China regions and then transported to the United States and is the most likely closest ancestor to the North American strains. Members of the genogroup 2a share only approximately 96.9% similarity to the prototype PEDV strain CV777 of genogroup 1a (Bridgen, et al. 1993; Huang et al. 2013; GenBank: AF353511.1). As such, the attenuated PEDV vaccines based on the historical CV777-derived G1a strains or DR13-derived G1b strains may be antigenically less related to the newly emergent Chinese and North American G2a PEDV strains.

A closely related North American PEDV isolate US/Colorado/2013 (GenBank Accession No: KF272920.1) has also been reported by Marthaler et al. 2013. Like the North American isolates above the complete PEDV genome of CO/13 has a nucleotide identity of 96.5 to 99.5% with other complete PEDV genomes available in GenBank, with the highest nucleotide identity (99.5%) with Chinese strain AH2012 (GenBank Accession No. KC210145). Chinese strain AH2012 is a member of the 2a genogroup. Comparison of the complete genome of North American isolate CO/13 to that of PEDV reference strain CV777, shows that CO/13 contains a 1-nucleotide insertion (at position 48) and deletions of 5 nucleotides in the 5′ UTR (at positions 73 and 83 to 86), while the spike gene contains insertions of 16 nucleotides (positions 20804, 20810 to 20820, 20843, and 21053 to 21055) and deletions of 7 nucleotides (positions 20853 and 21118 to 21124).

Other variants of PEDV have emerged, referred to as ‘INDEL’ strains which are often naturally attenuated compared to older prototype strains. These too may be used as vaccines wherein the virus is live attenuated, or inactivated. In such case only minimal further passaging may be needed to provide a safe vaccine attenuate. Exemplary vaccine viruses of the invention therefor also include those that have at least 95%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.41%, 97.42%, 97.43%, 97.44%, 97.45%, 97.46%, 97.47%, 97.48%, 97.49%, 97.5%, 97.51%, 97.52%, 97.53%, 97.54%, 97.55%, 97.56%, 97.57%, 97.58%, 97.59%, 97.6%, 97.61%, 97.62%, 97.63%, 97.64%, 97.65%, 97.66%, 97.67%, 97.68%, 97.69% 97.7%, 97.71%, 97.72%, 97.73%, 97.74%, 97.75%, 97.76%, 97.77%, 97.78%, 97.79%, 97.8%, 97.81%, 97.82%, 97.83%, 97.84%, 97.85%, 97.86%, 97.87%, 97.88%, 97.89%, 97.9%, 97.91%, 97.92%, 97.93%, 97.94%, 97.95%, 97.96%, 97.97%, 97.98%, 97.99%, 98%, 98.01%, 98.02%, 98.03%, 98.04%, 98.05%, 98.06%, 98.07%, 98.08%, 98.09%, 98.1%, 98.11%, 98.12%, 98.13%, 98.14%, 98.15%, 98.16%, 98.17%, 98.18%, 98.19%, 98.2%, 98.21%, 98.22%, 98.23%, 98.24%, 98.25%, 98.26%, 98.27%, 98.28%, 98.29%, 98.3%, 98.31%, 98.32%, 98.33%, 98.34%, 98.35%, 98.36%, 98.37%, 98.38%, 98.39%, 98.4%, 98.41%, 98.42%, 98.43%, 98.44%, 98.45%, 98.46%, 98.47%, 98.48%, 98.49%, 98.5%, 98.51%, 98.52%, 98.53%, 98.54%, 98.55%, 98.56%, 98.57%, 98.58%, 98.59%, 98.6%, 98.61%, 98.62%, 98.63%, 98.64%, 98.65%, 98.66%, 98.67%, 98.68%, 98.69%, 98.7%, 98.71%, 98.72%, 98.73%, 98.74%, 98.75%, 98.76%, 98.77%, 98.78%, 98.79%, 98.8%, 98.81%, 98.82%, 98.83%, 98.84%, 98.85%, 98.86%, 98.87%, 98.88%, 98.89%, 98.9%, 98.91%, 98.92%, 98.93%, 98.94%, 98.95%, 98.96%, 98.97%, 98.98%, 98.99%, 99%, 99.01%, 99.02%, 99.03%, 99.04%, 99.05%, 99.06%, 99.07%, 99.08%, 99.09%, 99.1%, 99.11%, 99.12%, 99.13%, 99.14%, 99.15%, 99.16%, 99.17%, 99.18%, 99.19%, 99.2%, 99.21%, 99.22%, 99.23%, 99.24%, 99.25%, 99.26%, 99.27%, 99.28%, 99.29%, 99.3%, 99.31%, 99.32%, 99.33%, 99.34%, 99.35%, 99.36%, 99.37%, 99.38%, 99.39%, 99.4%, 99.41%, 99.42%, 99.43%, 99.44%, 99.45%, 99.46%, 99.47%, 99.48%, 99.49%, 99.5%, 99.51%, 99.52%, 99.53%, 99.54%, 99.55%, 99.56%, 99.57%, 99.58%, 99.59%, 99.6%,99.61%, 99.62%, 99.63%, 99.64%, 99.65%, 99.66%, 99.67%, 99.68%, 99.69%, 99.7%, 99.71%, 99.72%, 99.73%, 99.74%, 99.75%, 99.76%, 99.77%, 99.78%, 99.79%, 99.8%, 99.81%, 99.82%, 99.83% 99.84%, 99.85%, 99.86%, 99.87%, 99.88%, 99.89%, 99.9%, 99.91%, 99.92%, 99.93%, 99.94%, 99.95%, 99.96%, 99.97%, 99.98% and 99.99% sequence identity or higher sequence identify with such strains, whether measured amino acid or encoding nucleotide sequence, for the spike protein or based on the full viral sequence.

The term “PEDV of North American origin” means a PEDV isolate comprising SEQ ID NO:29 and/or 33 and/or SEQ ID NO:32 and/or 36, and/or any PEDV isolates having at least 99% sequence identity to SEQ ID NO:29 and/or 33, and/or is at least 99% identical with the RNA equivalent of SEQ ID. NO:29 and/or 33, and/or a PEDV isolate in which a Spike protein is encoded by SEQ ID NO:30, 34, 46, or 52, and/or any PEDV isolate in which a Spike protein has at least 98% sequence identity to SEQ ID:30, 34, 46, or 52, and/or any PEDV isolate in which the expressed Spike protein has at least 90% homology with SEQ ID NO:31, 35, 47, 53.

The term “clade” as it is known in the art refers to a group consisting of an ancestor and all its descendants, a single “branch” in a phylogenetic tree. The ancestor may be, as an example an individual, a population or a species. A genogroup can include multiple clades, for example AH2012 is in a different clade than the North American isolates.

According to a further embodiment, the present invention also relates to a vector that comprises any of such nucleic acid molecules as described herein. In other words, the present invention relates to a vector, that includes the coding sequence of any such Spike, M, E, N PEDV protein, or part thereof. Preferably, said vector is an expression vector, which allows the expression of any such Spike, M, E, and/or N PEDV protein or part of the protein. Vectors according to the invention are those which are suitable for the transfection or infection of bacterial, yeast or animal cells, in vitro or in vivo.

Vectors and methods for making and/or using vectors (or recombinants) for expression can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018; Paoletti, “Applications of pox virus vectors to vaccination: An update, “PNAS USA 93:11349-11353, October 1996; Moss, “Genetically engineered poxviruses for recombinant gene expression, vaccination, and safety,” PNAS USA 93:11341-11348, October 1996; Smith et al., U.S. Pat. No. 4,745,051 (recombinant baculovirus); Richardson, C. D. (Editor), Methods in Molecular Biology 39, “Baculovirus Expression Protocols” (1995 Humana Press Inc.); Smith et al., “Production of Human Beta Interferon in Insect Cells Infected with a Baculovirus Expression Vector”, Molecular and Cellular Biology, December 1983, Vol. 3, No. 12, p. 2156-2165; Pennock et al., “Strong and Regulated Expression of Escherichia coli B-Galactosidase in Infect Cells with a Baculovirus vector, “Molecular and Cellular Biology March 1984, Vol. 4, No. 3, p. 406; EPA0 370 573; U.S. application No. 920,197, filed Oct. 16, 1986; EP Patent publication No. 265785; U.S. Pat. No. 4,769,331 (recombinant herpesvirus); Roizman, “The function of herpes simplex virus genes: A primer for genetic engineering of novel vectors,” PNAS USA 93:11307-11312, October 1996; Andreansky et al., “The application of genetically engineered herpes simplex viruses to the treatment of experimental brain tumors,” PNAS USA 93:11313-11318, October 1996; Robertson et al., “Epstein-Barr virus vectors for gene delivery to B lymphocytes”, PNAS USA 93:11334-11340, October 1996; Frolov et al., “Alphavirus-based expression vectors: Strategies and applications,” PNAS USA 93:11371-11377, October 1996; Kitson et al., J. Virol. 65, 3068-3075, 1991; U.S. Pat. Nos. 5,591,439, 5,552,143; WO 98/00166; allowed U.S. application Ser. Nos. 08/675,556, and 08/675,566 both filed Jul. 3, 1996 (recombinant adenovirus); Grunhaus et al., 1992, “Adenovirus as cloning vectors,” Seminars in Virology (Vol. 3) p. 237-52, 1993; Ballay et al. EMBO Journal, vol. 4, p. 3861-65, Graham, Tibtech 8, 85-87, April 1990; Prevec et al., J. Gen Virol. 70, 42434; PCT WO 91/11525; Felgner et al. (1994), J. Biol. Chem. 269, 2550-2561, Science, 259:1745-49, 1993; and McClements et al., “Immunization with DNA vaccines encoding glycoprotein D or glycoprotein B, alone or in combination, induces protective immunity in animal models of herpes simplex virus-2 disease”, PNAS USA 93:11414-11420, October 1996; and U.S. Pat. Nos. 5,591,639, 5,589,466, and 5,580,859, as well as WO 90/11092, WO93/19183, WO94/21797, WO95/11307, WO95/20660; Tang et al., Nature, and Furth et al., Analytical Biochemistry, relating to DNA expression vectors, inter alia. See also WO 98/33510; Ju et al., Diabetologia, 41:736-739, 1998 (lentiviral expression system); Sanford et al., U.S. Pat. No. 4,945,050; Fischbachet al. (Intracel); WO 90/01543; Robinson et al., Seminars in Immunology vol. 9, pp. 271-283 (1997), (DNA vector systems); Szoka et al., U.S. Pat. No. 4,394,448 (method of inserting DNA into living cells); McCormick et al., U.S. Pat. No. 5,677,178 (use of cytopathic viruses); and U.S. Pat. No. 5,928,913 (vectors for gene delivery); as well as other documents cited herein.

Preferred viral vectors include baculovirus such as BaculoGold (BD Biosciences Pharmingen, San Diego, Calif.), in particular provided that the production cells are insect cells.

Although the baculovirus expression system is preferred, it is understood by those of skill in the art that other expression systems will work for purposes of the present invention, namely the expression of HG into the supernatant of a cell culture. Such other expression systems may require the use of a signal sequence in order to cause H5 expression into the media.

### Effective Dose

The compounds described herein can be administered to a subject at therapeutically effective doses to prevent PDCoV and/or PEDV-associated diseases. The dosage will depend upon the host receiving the vaccine as well as factors such as the size, weight, and age of the host.

The precise amount of immunogenic composition of the invention to be employed in a formulation will depend on the route of administration and the nature of the subject (e.g., age, size, stage/level of disease), and should be decided according to the judgment of the practitioner and each subject's circumstances according to standard clinical techniques. An effective immunizing amount is that amount sufficient to treat or prevent a PDCoV and/or PEDV infectious disease in a subject.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response. Test subjects may include animals such as pigs, mice, hamsters, dogs, cats, rabbits, cows, horses, sheep, poultry (e.g. chickens, ducks, geese, and turkeys), and humans.

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, virus neutralization, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use in animals. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutic may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

In addition, the therapeutic can be assessed by measuring the level of the molecule against which the antibody is directed in the animal model or human subject at suitable time intervals before, during, or after therapy. Any change or absence of change in the amount of the molecule can be identified and correlated with the effect of the treatment on the subject. The level of the molecule can be determined by any method known in the art.

After vaccination of an animal with PDCoV and/or PEDV using the methods and compositions of the present invention, any binding assay known in the art can be used to assess the binding between the resulting antibody and the particular molecule. These assays may also be performed to select antibodies that exhibit a higher affinity or specificity for the particular antigen.

### Administration to a Subject

Preferred routes of administration include but are not limited to intranasal, oral, intradermal, and intramuscular. The skilled artisan will recognize that compositions of the invention may also be administered in one, two or more doses, as well as, by other routes of administration. For example, such other routes include subcutaneously, intracutaneously, intravenously, intravascularly, intraarterially, intraperitnoeally, intrathecally, intratracheally, intracutaneously, intracardially, intralobally, intramedullarly, intrapulmonarily, and intravaginally. Depending on the desired duration and effectiveness of the treatment, the compositions according to the invention may be administered once or several times, also intermittently, for instance on a daily basis for several days, weeks or months and in different dosages.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Isolation and Production of PEDV Strain

To produce the porcine epidemic diarrhea virus vaccine, killed virus, a master seed culture of a PEDV (isolate) was first produced. From this master seed, a culture of PEDV was grown and then inactivated. The inactivated virus culture was then mixed with an adjuvant in order to produce the porcine epidemic diarrhea virus vaccine. The following method was used to produce the porcine epidemic diarrhea virus vaccine.

Animals or tissues from animals exhibiting extreme diarrhea were acquired in 2013. Homogenates from mucosal scrapings were generated from these animals filtered through a 0.2 micron syringe filter and the filtrate was used to inoculate African Green Monkey kidney cells (VERO). Virus was grown in the presence of PEDV maintenance media containing modified MEM, porcine trypsin, tryptose phosphate broth, yeast extract and HEPES buffer. Virus growth was evaluated and visualized by checking for characteristic syncytia formation and fusion of cell monolayer. CPE positive material was subjected to sequencing using Illumina-based MISEQ® sequencer technology.

In order to produce the PEDV master seed virus culture (“PEDV MSV”), porcine epidemic diarrhea virus strain (isolate) (PEDV isolate) was isolated in BI VERO cells and passed a total nineteen times in BI VERO cells and then virus was grown in 2013 EU VERO cells till passage 30. The 30^{th} passage of the virus was diluted and put down as the master seed virus designated PEDV KV-1251-125-10-OK.

From the master seed virus, a culture of PEDV (KV-1251-125-10-OK, to be referred to herein as “125-10”) was produced by infecting 2013 EU VERO cells with PEDV KV-1251-125-10-OK MSV in PEDV maintenance media containing modified Minimal Essential Media, porcine trypsin (10 μg/ml), tryptose phosphate broth (0.3%), yeast extract (0.02%) and 1M HEPES buffer (2.5%) The 2013 EU VERO cells were typically infected with the PEDV (125-10) MSV at a minimum dose of 10⁴ TCID₅₀/850 cm² roller bottle. Such cultures can be grown in sterile disposable roller bottles or on microcarrier beads. The culture was incubated at 36° C.±2° C. for 24 to 48 hours until cytopathic effect (“CPE”) was observed. Typically, characteristic syncytia can be seen within 12 hours of infection, syncytia expand and cell monolayer fuses from 24-48 hours followed by sloughing of cells. During incubation, the culture was monitored for PEDV induced CPE to ensure a pure PEDV strain. If atypical CPE was observed or any macroscopic or microscopic evidence of contamination existed, the culture was discarded. Pure virus culture was aseptically harvested into sterile polypropylene carboys. Virus was freeze thawed to release cell associated virions and was clarified by centrifugation or by filtration through filters of 0.45 microns followed by 0.2 microns. Bulk virus harvest fluids were tested to ensure the absence of mycoplasma prior to inactivation. Harvested fluids which were not immediately inactivated were stored at −70° C. or below.

The volume of harvested fluids is determined and the temperature of the fluids is brought to 36±2° C. A 0.4 M solution of 2-bromoethyleneamine (BEA) is mixed with a stock solution of 0.3 N NaOH to generate a binary ethyleneimine (BEI) stock solution which is then added to the harvest fluids to give a final concentration of BEI of 5 mM. The fluids are stirred continuously for a minimum of 24 hours. A 1.0 M sodium thiosulfate solution to give a final minimum concentration of 5 mM is added to neutralize any residual BEI. The inactivated fluids can be stored at −70±3° C. for long term storage or at 4±3° C. for short term.

After treatment with BEI, the culture was tested for its ability to induce CPE typical of PEDV to ensure inactivation of the virus. This task was accomplished by passing the BEI treated viral fluids over Vero cells and checking the Vero cells for any viral infection. The BEI treated culture fluids were typically stored at −70° C. or below until the inactivation assay had been completed.

The inactivated virus was formulated as an adjuvanted vaccine by thoroughly blending the inactivated PEDV culture with adjuvant EMULSIGEN®-BCL at a 20% inclusion rate to form a bulk serial. The bulk serial was maintained at 2-8° C. until being transferred into vials containing either one or ten doses (@2.0 ml per dose).

### Example 2: Isolation and Production of Porcine Deltacoronavirus Strain

The Porcine Deltacoronavirus (PDCoV) was purchased from the National Veterinary Service laboratory (NVSL), Ames, IA. This virus was isolated at NVSL from a swine intestinal sample from Illinois in 2014. The virus was isolated on Swine testes (ST) cells in serum free media in the presence of 5 μg/mL TPCK trypsin. The original virus went through two rounds of plaque purification at NVSL and the tenth passage of the virus was utilized as the starting material.

The pass 10 virus was inoculated and adapted to swine testes (ST) cells at BI with PDCoV maintenance media that contained Modified Minimal Essential Media with HEPES, tryptose phosphate broth, yeast extract and porcine trypsin. The cells were incubated at 24-48 hours at 37° C. with 5% CO2 and observed for evidence of PDCoV-induced cytopathic effect. When the CPE was complete and cells had sloughed off, the flasks were freeze-thawed once and the lysate was harvested. The harvested material was then inoculated onto a fresh monolayer for continued passage. Passage continued in this manner through pass 12. Virus harvested from pass 12 was inoculated and adapted to an alternate, derivative swine testes (ST) cell lineage designated “AI-ST 2015” cells. Four more passages of the virus were carried out in AI-ST 2015cells in the same manner as passes 11 and 12 to get P16 virus. P16 virus was inoculated into roller bottles yield the final “P17” master seed virus isolate which was harvested, filtered through 0.2 μm filter, pooled to obtain a bulk virus stock and stored below −60° C. The average titer of the master seed virus “P17” was determined to be 4.91log10 FAID50 per mL.

### Example 3: Genome Sequence Analysis of PEDV Isolate and PDCoV Isolate

### Sample Preparation and Analysis:

Prior to extraction virus tissue culture supernatants were pre-treated with a cocktail of DNase and RNase to remove residual host cell genomic nucleic acids. Viral genomic RNA was then extracted from the nuclease-treated samples using the RNEASY® viral RNA extraction kit (Qiagen, Cat #52906). Post extraction, samples were again treated with DNase to further enrich for viral genomic RNA. Subsequently, viral genomic RNA was converted to double stranded cDNA (ds cDNA) through randomly primed reverse transcription and Klenow fragment treatment. The ds cDNA products were then used to generate a library for Illumina MISEQ® sequencer-based sequencing using the NEXTERA®XT library preparation kit (Cat #FC-131-1024). Each sample was barcoded with unique tags on both the 5′- and 3′-ends to minimize the chances of bioinformatic mis-binning. This library was run on the MISEQ® sequencer using the 500-cycle kit (Cat #MS-102-2003) and data was analyzed using a combination of NextGene (version 2.3.4) and Sequencher software (version 5.1). High quality sequences were selected as those containing a median Q-score of greater than 25 and trimmed with a cut-off of no more than three uncalled bases at 3′-end or 3-consecutive bases with Q-score measuring less than 16. The sequences were then assembled de novo using criteria of 85% or greater match over a 35 bp stretch to generate a putative full genome for each isolate. The putative complete genome sequence for each was then verified by template-based alignment to verify single nucleotide polymorphisms (SNP) or variable small insertions/deletions.

For PEDV isolate 1251-125-10, a total of 570,253 sequences were generated with an average length of 13 6bp after trimming of low quality data. Of those sequences; 484,247 (84.9%) assembled into a single contig 27,995 bp long which through BLASTn analysis revealed strong identity to the single-stranded RNA Alphacoronavirus PEDV. A total of 11 positions exhibited polymorphism at either a single nucleotide or a small insertion/deletion, these positions are listed in TABLE 1.

TABLE 1
Polymorphic Residues in Isolate 1251-125-10 “125-10”

| | Residue | |
|---|---|---|
| Position | Frequencies | Gene |
| 3,315 | T (51%) | ORF1A/B |
| | A (49%) | |
| 3,423-3,426 | DEL (50%) | ORF1A/B |
| | TTA (50%) | |
| 9,425 | C (64%) | ORF1A/B |
| | T (36%) | |
| 10,136 | T (52%) | ORF1A/B |
| | A (48%) | |
| 14,416 | A (69%) | ORF1A/B |
| | G (31%) | |
| 18,179 | C (73%) | ORF1A/B |
| | T (27%) | |
| 19,100 | C (73%) | ORF1A/B |
| | T (27%) | |
| 23,101 | G (63%) | Spike |
| | A (37%) | |
| 25,057 | T (59%) | Spike |
| | 10 bp INS | |
| | (41%) | |
| 25,165-25,169 | TTATG | ORF3 |
| | (74%) | |
| | DEL (26%) | |
| 27,510 | C (73%) | ORF3 |
| | T (27%) | |

The putative complete/near-complete PEDV genome of 1251-125-10 (SEQ ID NO:29) was aligned to the closest Chinese AH2012 (GenBank Accession No: KC210145) and North American Colorado 2013 isolate of PEDV (GenBank Accession No: AGO58924). The identities to both isolates exceed 99.2% indicating very close relation to both strains, both in genogroup 2a.

Next, the immunogenic spike protein sequence was examined for protein identity/similarity to the larger GenBank repository of PEDV spike proteins. Again, the closest GenBank isolate submitted was derived from the North American Colorado 2013 strain deposited by the University of Minnesota Veterinary diagnostic laboratory (GenBank Accession No: AGO58924) exhibiting over 99.5% identity (1380/1386 identical amino acids)(SEQ ID NO:X). Of the 6 amino acid changes, 1 was due to the polymorphism at position 23,101 which would encode either CGA (Arg) in the majority or CAA (Gln) in minority at position 838. The North American Colorado 2013 strain contains a Gln at this position.

For PDCoV, 12 individually isolated samples were sequenced. Materials were extracted following viral particle protected nucleic acid digestion (RNase+DNase) in order to enrich for viral RNA. Post-extraction, nucleic acids treated with DNase to enrich for RNA. Double-stranded cDNA was generated through reverse transcription and klenow treatment using priming with random hexamers. These products were then used for library generation.

Samples were processed for MISEQ® sequencer based sequencing through library generation using the NEXTERA® XT library preparation kit (Illumina, Cat #FC-131-1024). Each sample was barcoded with unique tags on both the 5′- and 3′-ends to minimize the chances of bioinformatic mis-binning. The library was run on the MISEQ™ sequencer using the 500-cycle kit (Illumina, Cat #MS-102-2003) and data was analyzed using a combination of NEXTGENE® sequencing software (SoftGenetics, LLC, version 2.3.4.2) and SEQUENCER® software (Gene Codes Corp., version 5.1). High quality sequences were selected as those containing a median Q-score of greater than 25 and trimmed with a cut-off of no more than 3uncalled bases at 3′-end or 3-consecutive bases with Q-score measuring less than 16. These sequences were both assembled de novo and aligned to reference files using criteria of 85% or greater match over a 35 bp stretch. Reference sequences for alignment were from de novo sequence assemblies or MISEQ® sequencer derived references for Carthage and NVSL PDCoV strains (NSVL, porcine Deltacoronavirus strain USA/IL/2014/026PDV_P11, Genbank Accession No. KP981395.1).

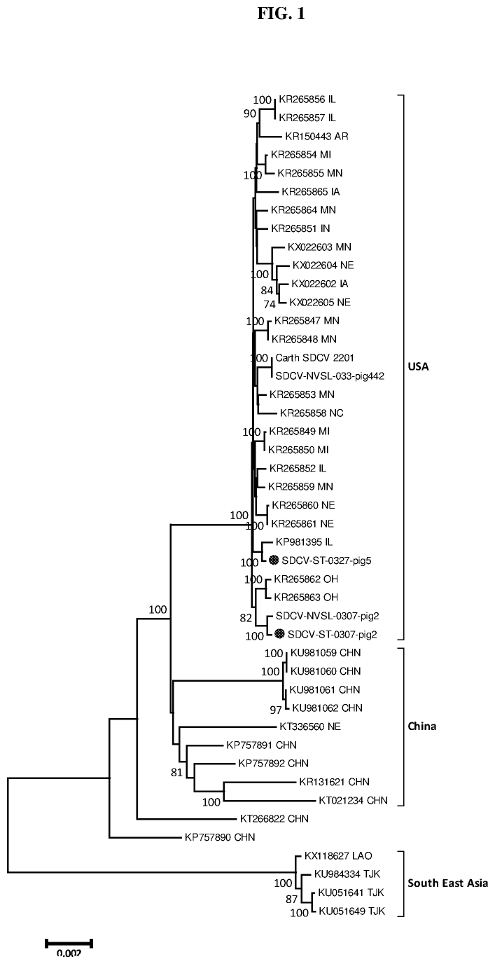

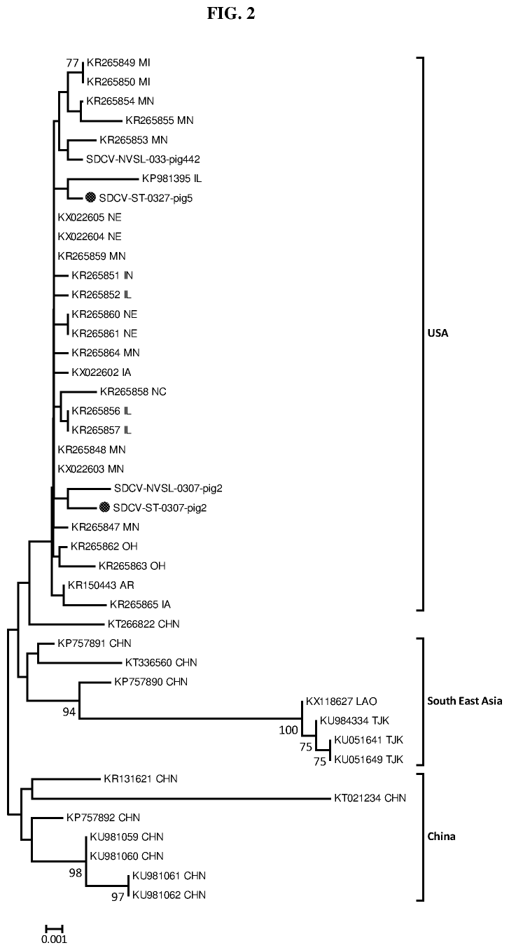

Overall, the genetic similarity between each of the BI virus isolates and other strains (“Carthage” original isolate and NVSL strain) was high (>99.8% identity) and differences between variants of NVSL and BI-ST cell grown viruses exhibiting even fewer changes (>99.95% identity) (TABLE 2) See also FIGS. 1 and 2 schematically showing a complete genome nucleotide neighbor-joining tree for PDCoV isolates/strains, and a Spike amino acid neighbor-joining tree for PDCoV isolates/strains, respectively. Scale indicates p-distance.

The similarities between variants of NVSL and BI-ST cell grown viruses are most likely due to the shared origin of the some of the samples. One notable sequence difference is the presence of an in-frame 9 bp deletion in spike (amino acid positions 229-231) within the cultured PDCoV_2201-1 isolates; but not observed in either the original homogenate or materials from pigs. This position appears to be a “hot spot” for IN/DEL variability as the BI PDCoV-5.0327 isolate, which has no known connection to the “Carthage” isolate, also exhibits the identical deletion in the “BI-ST” cell line, but not the NVSL-ST cell line. These changes may be indicative of tissue culture adaptation of the virus.

TABLE 2
genetic similarity between each of the BI virus isolates.

| | Full Genome Nucleotide Distance | | | SPIKE Protein Amino Acid Distance | | |
|---|---|---|---|---|---|---|
| | PDCoVST0 | PDCoVST0 | BIVI_NVSL | PDCoVST0 | PDCoVST0 | BIVI_NVSL |
| | 2.0307 | 5.0327 | isolate | 2.0307 | 5.0327 | isolate |
| KP757890_CHN | 98.97% | 99.05% | 98.89% | 98.97% | 99.05% | 98.62% |
| KP757891_CHN | 99.22% | 99.40% | 99.25% | 99.22% | 99.40% | 98.88% |
| KP757892_CHN | 99.14% | 99.22% | 99.19% | 99.14% | 99.22% | 98.79% |
| KP981395_IL | 99.22% | 99.48% | 99.94% | 99.22% | 99.48% | 99.91% |
| KR131621_CHN | 98.79% | 98.88% | 98.91% | 98.79% | 98.88% | 98.45% |
| KR150443_AR | 99.66% | 99.74% | 99.76% | 99.66% | 99.74% | 99.31% |
| KR265847_MN | 99.57% | 99.66% | 99.82% | 99.57% | 99.66% | 99.22% |
| KR265848_MN | 99.66% | 99.74% | 99.83% | 99.66% | 99.74% | 99.31% |
| KR265849_MI | 99.57% | 99.66% | 99.85% | 99.57% | 99.66% | 99.22% |
| KR265850_MI | 99.57% | 99.66% | 99.85% | 99.57% | 99.66% | 99.22% |
| KR265851_IN | 99.66% | 99.74% | 99.84% | 99.66% | 99.74% | 99.31% |
| KR265852_IL | 99.66% | 99.74% | 99.84% | 99.66% | 99.74% | 99.31% |
| KR265853_MN | 99.48% | 99.57% | 99.84% | 99.48% | 99.57% | 99.14% |
| KR265854_MI | 99.57% | 99.66% | 99.83% | 99.57% | 99.66% | 99.22% |
| KR265855_MN | 99.31% | 99.40% | 99.79% | 99.31% | 99.40% | 98.97% |
| KR265856_IL | 99.66% | 99.74% | 99.80% | 99.66% | 99.74% | 99.31% |
| KR265857_IL | 99.66% | 99.74% | 99.80% | 99.66% | 99.74% | 99.31% |
| KR265858_NC | 99.48% | 99.57% | 99.79% | 99.48% | 99.57% | 99.14% |
| KR265859_MN | 99.74% | 99.83% | 99.85% | 99.74% | 99.83% | 99.40% |
| KR265860_NE | 99.66% | 99.74% | 99.84% | 99.66% | 99.74% | 99.31% |
| KR265861_NE | 99.66% | 99.74% | 99.84% | 99.66% | 99.74% | 99.31% |
| KR265862_OH | 99.66% | 99.74% | 99.80% | 99.66% | 99.74% | 99.31% |
| KR265863_OH | 99.40% | 99.48% | 99.80% | 99.40% | 99.48% | 99.05% |
| KR265864_MN | 99.66% | 99.74% | 99.83% | 99.66% | 99.74% | 99.31% |
| KR265865_IA | 99.40% | 99.48% | 99.78% | 99.40% | 99.48% | 99.05% |
| KT021234_CHN | 97.50% | 97.59% | 98.82% | 97.50% | 97.59% | 97.16% |
| KT266822_CHN | 99.14% | 99.22% | 98.90% | 99.14% | 99.22% | 98.79% |
| KT336560_NE | 98.88% | 98.97% | 99.00% | 98.88% | 98.97% | 98.53% |
| KU051641_TJK | 97.59% | 97.59% | 97.37% | 97.59% | 97.59% | 97.16% |
| KU051649_TJK | 97.50% | 97.50% | 97.36% | 97.50% | 97.50% | 97.07% |
| KU981059_CHN | 98.97% | 99.05% | 98.96% | 98.97% | 99.05% | 98.62% |
| KU981060_CHN | 98.97% | 99.05% | 98.96% | 98.97% | 99.05% | 98.62% |
| KU981061_CHN | 98.71% | 98.79% | 98.96% | 98.71% | 98.79% | 98.36% |
| KU981062_CHN | 98.71% | 98.79% | 98.95% | 98.71% | 98.79% | 98.36% |
| KU984334_TJK | 97.67% | 97.67% | 97.39% | 97.67% | 97.67% | 97.24% |
| KX022602_IA | 99.57% | 99.74% | 99.75% | 99.57% | 99.74% | 99.22% |
| KX022603_MN | 99.66% | 99.83% | 99.76% | 99.66% | 99.83% | 99.31% |
| KX022604_NE | 99.66% | 99.83% | 99.74% | 99.66% | 99.83% | 99.31% |
| KX022605_NE | 99.66% | 99.83% | 99.76% | 99.66% | 99.83% | 99.31% |
| KX118627_LAO | 97.76% | 97.76% | 97.43% | 97.76% | 97.76% | 97.33% |
| PDCoV-NVSL-0307- | 99.57% | 99.48% | 99.83% | 99.57% | 99.48% | 99.05% |
| pig2 | | | | | | |
| PDCoV-NVSL-033- | 99.57% | 99.66% | 99.83% | 99.57% | 99.66% | 99.22% |
| pig442 | | | | | | |
| PDCoV-ST-0307-pig2 | | 99.57% | 99.84% | | 99.57% | 99.14% |
| PDCoV-ST-0327-pig5 | | | 99.94% | | | 99.40% |

bottom 10% lowest values in identity (most divergent).
Top 10% highest in identity (least divergent).

### Example 4: Inactivation of Whole PEDV and PDCoV Isolates

Each lot of PEDV virus or pool is tested for inactivation by passage in VERO cells. Seventy five cm² of 24 hour cell culture are inoculated with 2.0 mL of inactivated PEDV fluids and maintained at 36±3° C. for 48 hours. One flask of VERO cells remains uninoculated. For positive virus controls one culture of VERO cells is inoculated with a positive control PEDV. At the end of the incubation period, the cell monolayers are examined for CPE typical of PEDV. The material is frozen and thawed three times and then 2 ml of each material is inoculated onto one day old VERO cells. The culture should be maintained at 37±2° C. for 48 hours. Following the second passage, a third passage is performed. After incubation and passage, the absence of virus-infected cells in the BEI treated viral fluids as determined by lack of immunofluorescence staining constitutes a satisfactory inactivation test. The control cells inoculated with the positive control virus shall show CPE typical of PEDV and the uninoculated flask shall show no evidence of PEDV CPE.

PDCoV viral harvest is inactivated with 5 mM BEI for 24 hours at 37° C. Following inactivation, the harvest is neutralized with sodium thiosulfate. Inactivation was confirmed by TCID50 evaluation of the inactivated harvest. No live virus was detected in the inactivated material. Inactivated, neutralized viral harvest is concentrated using a 10 kd hollow fiber PES cartridge (GC Healthcare cat #UFP-10-C-3MA). Concentrated antigen is aseptically diluted with modified minimal essential media (final concentration=10×). A portion of the diluted antigen is combined with EMULSIGEN D® (MVP Laboratories, item #10005408) to achieve a 12.5% formulation.

### Example 5

### Construction of a Recombinant

### Baculoviruses Coding for and Expressing PDCoV Spike Antigens

### Preparation of PDCoV S1-IgG2a Fc Baculovirus

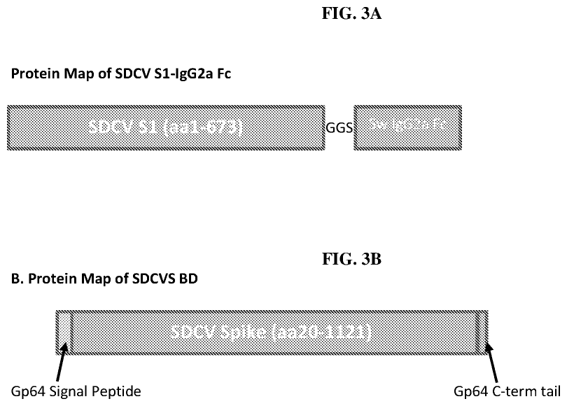

The S1 region of porcine Deltacoronavirus (PDCoV) spike (aa 1-673) was amplified by PCR from plasmid DNA using primers P2967167A (SEQ ID NO:13) and P2967167B (SEQ ID NO:14). The Fc region of swine IgG2a including a short GGS linker and the hinge region was amplified from plasmid DNA using primers P2967167C (SEQ ID NO:15) and P2968014E (SEQ ID NO:16). The two fragments were fused by overlap-extension PCR using primers P2967167A (SEQ ID NO:13) and P2968014E (SEQ ID NO:16) to generate the PDCoV S1-IgG2a Fc coding sequence (SEQ ID NO:17) containing a Kozak consensus sequence immediately 5′ of the start codon. See the schematic diagram in FIG. 3.A. The final coding sequence was flanked by BamHI and NotI restriction sites to facilitate cloning into baculovirus transfer plasmid pVL1393. Once completed, plasmid pVL1393-PDCoV S1-IgG2a Fc was used with linearized BaculoGold baculovirus DNA to transfect Sf9 insect cells to produce recombinant baculovirus.

### Preparation of PDCoVS BD Baculodisplay Baculovirus

The PDCoV Spike gene from porcine Deltacoronavirus (PDCoV) was cloned in two overlapping fragments (N-term and C-term) from plasmid DNA. The N-terminal fragment was amplified using primers P2967002C (SEQ ID NO:23) and P2967002D (SEQ ID NO:24) while the C-term fragment was amplified using primers P2967002E (SEQ ID NO:25) and P2967002F (SEQ ID NO:26). The N-terminal and C-terminal fragments were amplified so as to remove the native signal sequence and C-terminal tail. The N-term fragment was fused with the Autographa californica nucleopolyhedrovirus (AcNPV) gp64 signal sequence (02967002A) (SEQ ID NO:21) by overlap-extension PCR (OE-PCR) using primers P290194A (SEQ ID NO:19) and P2967002D (SEQ ID NO:20). The C-term fragment was fused with the AcNPV gp64 C-terminal tail coding sequence (02967002B) (SEQ ID NO:22) by OE-PCR using primers P290194B (SEQ ID NO:20) and P2967002E (SEQ ID NO:25). The two resulting fragments were fused by OE-PCR using primers P290194A (SEQ ID NO:19) and P290194B (SEQ ID NO:20) to generate the PDCoV spike BaculoDisplay (PDCoVS BD) coding sequence (SEQ ID NO:27) containing a Kozak consensus sequence immediately 5′ of the start codon. See the schematic diagram in FIG. 3B. The final coding sequence was flanked by BamHI and NotI restriction sites to facilitate cloning into baculovirus transfer plasmid pVL1393. Once completed, plasmid pVL1393-PDCoVS BD was used with linearized BaculoGold baculovirus DNA to transfect Sf9 insect cells to produce recombinant baculovirus.

### Example 6

### Preparation of Pharmaceutical Compositions (Vaccines)

### Comprising PEDV Spike Antigens

For the inactivated PEDV material, PEDV viral harvest was inactivated for a minimum of 24 hours using 5 mM BEI, clarified and 0.45 μm filtered.

After neutralization various adjuvants were added and the following vaccine/pharmaceutical compositions were generated.

### Example 7

### Preparation of Pharmaceutical Compositions (Vaccines)

### Comprising PDCoV Spike Antigens

Batches of PDCoV antigen (BacluloS-BD-PDCoV and BaculoS-FC-PDCoV) were grown in 3L spinners. For infection, SF+ cells were inoculated with the virus at an approximate MOI of between 1.0-2.1. Flasks were incubated at 27° C. with agitation set at 100 rpm. Harvest was initiated at five days post-infection. At the time of harvest cell viability was between 24% and 26% and viable cells/mL was between 0.31×10⁶ and 0.36×10⁶. Harvest fluids were clarified by centrifugation at 10,000×g for 10 minutes and 0.2 μm filtered. Clarified harvest fluids are combined with EMULSIGEN® D to achieve a 12.5% formulation.

### Example 8

### Inoculation of Pigs with Inactivated PDCoV and Baculovirus Spike Vaccine and Assessment of the Serological Response

The study objective was to evaluate the serological response of PDCoV vaccine prototypes in conventional piglets. Several prototype vaccines in which the spike protein of PDCoV is expressed in various backbones have been generated. In addition, a whole cell inactivated viral preparation has been generated.

See TABLE 3 below for an explanation of the experimental groups. Groups 1-4 were randomized and held in the same room. Additionally, strict control animal (Group 5) weaned at approximately four weeks of age were included in the current study. On DO, pigs were randomized into four groups and administered a 2 mL dose of each of the prototype PDCoV vaccines or a placebo. On D21, pigs received a second, or booster administration of each of the prototype vaccines. Serum and oral fluids were collected from the pigs prior to administration of the treatment at each vaccination and on D35. Samples were assayed for evidence of seroconversion. General health observations were recorded by room daily. Injection sites were observed for reactions for a minimum of three days following administration of the vaccine.

TABLE 3
Experimental Groups

| Group | N (Pigs) | Vaccine |
|---|---|---|
| 1 | 10 | BaculoS-BD-PDCoV |
| 2 | 11 | Whole-cell PDCoV |
| 3 | 11 | Placebo |
| 4 | 10 | BaculoS-FC-PDCoV |

### Serological Response Following Vaccination as Detected by IFA

### Indirect Immunofluorescent Assay (IFA)

Two-fold serial dilutions from 1:40 to 1:320 were made of each sample in 1× phosphate buffered saline (PBS; Gibco c #10010-023). 100 μl of each diluted sample was added to PDCoV infected plates and incubated for one hour at 37° C. Following incubation, serum was removed and monolayers were washed two times with 1×PBS. Cells were then stained using 10 μl/well of a 1:100 dilution of FITC-conjugated-goat-anti-swine-IgG antibody. Titers were reported as the greatest serum dilution showing PDCoV staining in comparison to non-infected control wells.

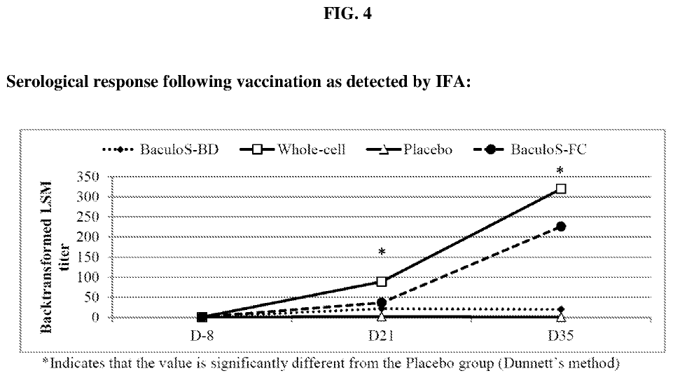

The serological response at D-8, D21 and D35 are presented by group in TABLE 4 below. Least square means and frequency of detection by day and group is presented in TABLE 5. No animals had a detectable serological response prior to vaccination. (See also FIG. 4).

Following a signal vaccination, 20-73% of animals had a response with animals vaccinated with BaculoS-BD prototype. By D35, all animals vaccinated with either the Whole-cell or BaculoS-FC prototypes had a detectable response. Only 50% of animals vaccinated with the BaculoS-BD prototype had a detectable response by D35.

No significant differences were observed at D-8 between the serological responses in pigs vaccinated with a prototype or a placebo. By D21 and 35, the serological response was higher in animals receiving a prototype vaccine (p<0.002 for all groups at all days). Numerically, the BaculoS-FC and Whole-cell prototype vaccines appeared to stimulate higher IgG responses by D35 in comparison to the BaculoSBD construct.

TABLE 4
Serological Data by Day and Group as measured by IFA

| | N | | D-8 | | D21 | | D35 | |
|---|---|---|---|---|---|---|---|---|
| Group | (Pigs) | Vaccine | LSM | % | LSM | % | LSM | % |
| 1 | 10 | BaculoS-BD- | 1 | 0 | 22* | 20 | 20* | 50 |
| | | PDCoV | | | | | | |
| 2 | 11 | Whole-cell | 1 | 0 | 89* | 73 | 320* | 100 |
| | | PDCoV | | | | | | |
| 3 | 11 | Placebo | 1 | 0 | 1 | 0 | 2 | 0 |
| 4 | 10 | BaculoS-FC- | 1 | 0 | 36* | 40 | 226* | 100 |
| | | PDCoV | | | | | | |

*Indicates that the value is significantly different from the Placebo group (Dunnett's method)

### Serological Response Following Vaccination as Detected by S1-IgG2a-Fc Based ELISA

### PDCoV IgG ELISA

Purified PDCoV-S1-IgG2aFc antigen (BIVI L #3091-141; 0.2 mg/mL) was diluted 1:6666.67 incarbonate buffer (BIVI L #3144-180). ELISA plates (high binding 96 well plates; Greiner cat #655061) were coated with 100 μl/well of diluted antigen and incubated overnight at 4° C. Plates were washed five times with 300 μl/well of TBST (0.05% Tween 20) using an automated plate washer. Following the wash step, 150 μl of blocking solution (Casein blocker, Thermo catalog #37532) was added per well and the plates were incubated at 15-30° C. for one hour. Initial serum dilutions were prepared in casein blocker at a dilution of 1:200 (negative control and test samples). Following incubation, the casein blocker was removed from the plate and serial two-fold dilutions (made in casein blocker) of the test samples were made from 1:200 through 12800. Serial two-fold dilutions of the negative control were made from 1:200 through 1:204800. Dilutions were prepared on the plate with an end volume of 100 μl/well. Plates were incubated at 37° C. for one hour with shaking (100 rpm). Following incubation, plates were washed as described previously. A 1:2000 dilution (in casein blocker) of a HRP conjugated goat-anti-swine-IgG (H+L) (Jackson ImmunoResearch catalog #114-035-003) was prepared and 100 μl was added to each well. The plates were incubated at 37° C. for one hour with shaking at 100 rpm then washed as previously described. For detection, 100 μl/well of prepared TMB substrate (KPL cat #50-76-00) was added per well. The plates were incubated at 15-30° C. for 10minutes. The reaction was stopped with 100 μl/well of 1N HCl and immediately read at 450 nm with an automated plate reader.

Results were generated through the following calculations. First, the mean and standard deviation optical density (OD) readings from the final five negative serum dilution wells were calculated. A cut-off was then established as the mean plus three standard deviations. Each sample optical density reading (at their initial dilution) was divided by the background cut-off for that plate. Data was reported as the signal: background ratio.

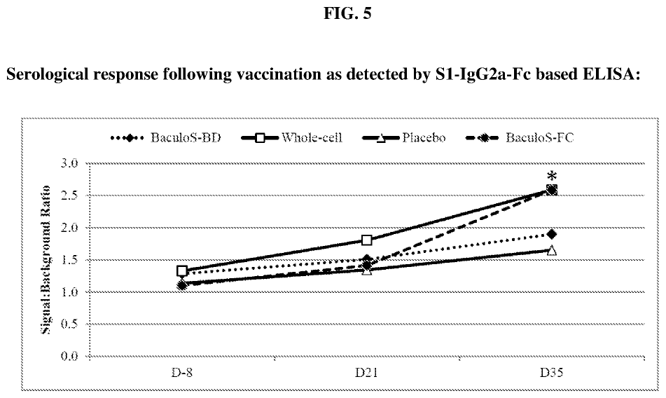

The serological response at D-8, D21 and D35 are presented by group. Least square means and frequency of detection by day and group is presented in TABLE 5 (See also FIG. 5). No animals had a detectable serological response prior to vaccination.

Following a second vaccination, 40-45% of animals had a response regardless of group. By D35, all animals vaccinated with either the Whole-cell or BaculoS-FC prototypes had a detectable response. No significant differences were observed at D8 or D21 between the serological responses in pigs vaccinated with a prototype or a placebo. By D35, the serological response was higher in animals receiving a prototype vaccine (p=0.0039, <0.0001 or <0.0001 for Groups 1, 2 and 4 respectively). Similar to the IFA results, the BaculoS-FC and Whole-cell prototype vaccines stimulated higher IgG responses by D35 in comparison to the BaculoS-BD construct.

TABLE 5
Serological Data by Day and Group as measured by IgG ELISA

| | N | | D-8 | | D21 | | D35 | |
|---|---|---|---|---|---|---|---|---|
| Group | (Pigs) | Vaccine | LSM | % | LSM | % | LSM | % |
| 1 | 10 | BaculoS-BD- | 1.2840 | 0 | 1.5068 | 40 | 1.8965* | 70 |
| | | PDCoV | | | | | | |
| 2 | 11 | Whole-cell | 1.3275 | 0 | 1.8040 | 45 | 2.5857* | 100 |
| | | PDCoV | | | | | | |
| 3 | 11 | Placebo | 1.1350 | 0 | 1.3409 | 9 | 1.6487 | 9 |
| 4 | 10 | BaculoS-FC- | 1.0997 | 0 | 1.4120 | 40 | 2.5857* | 100 |
| | | PDCoV | | | | | | |

*Indicates that the value is significantly different from the Placebo group (Dunnett's method)

### Example 9

### Efficacy of PEDV Baculovirus Vaccines

The following study evaluated the serological response to vaccination with two 2-mL does of a killed Porcine Epidemic Diarrhea Virus (PEDV) Vaccine, or a baculovirus construct vaccine, as measured after administration of either vaccine to pigs at three weeks of age. The primary outcome was serology tested by fluorescent focus neutralization (FFN) for serum samples collected following vaccination in the treated pigs.

The study groups included: T01=PBS (n=10); T02=6.93 log TCID 50/ml BEI PEDV+20% EMULSIGEN®-BCL (n=20); T03=Baculovirus with PEDV Spike Ag (n=9); 6× Concentrated Baculovirus with PEDV Spike Ag (n=10); Trypsin Baculovirus with PEDV Spike Ag (n=10); and Killed Positive Control vaccine conditionally licensed (POS CON) (n=10). On D0, pigs were administered the 2-mL treatment intramuscularly in the right neck. A second treatment was administered on D14 in the left neck for T01-T05 and on D21 for T06.

### Preparation of PEDV2a-BD

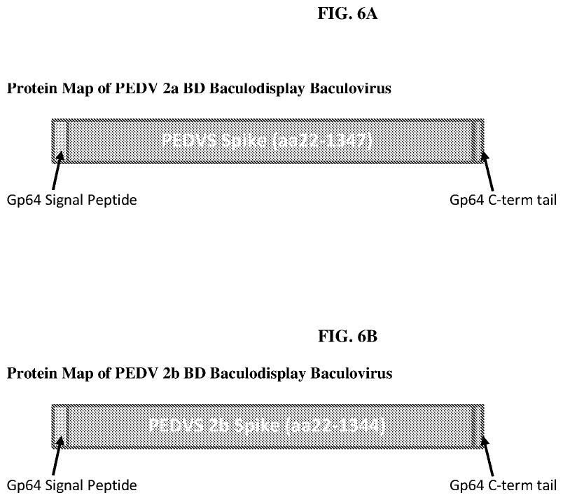

In this study, the spike gene from porcine epidemic diarrhea virus (PEDV) 2a was cloned in two overlapping fragments (N-term and C-term) from plasmid DNA. The N-terminal fragment was amplified using primers P1360110A (SEQ ID NO:37 and PEDV-S2-R (SEQ ID NO:38) while the C-term fragment was amplified using primers PEDV-S1-F (SEQ ID NO:39) and P1360110B (SEQ ID NO:40). The N-terminal and C-terminal fragments were amplified so as to remove the native signal sequence and C-terminal tail. The N-term fragment was fused with the Autographa californica nucleopolyhedrovirus (AcNPV) gp64 signal sequence (O1360110C) (SEQ ID NO:41) by overlap-extension PCR (OE-PCR) using primers P290194A (SEQ ID NO:45) and PEDV-S2-R (SEQ ID NO:38). The C-term fragment was fused with the AcNPV gp64 C-terminal tail coding sequence (01360110D) (SEQ ID NO:42) by OE-PCR using primers P290194B (SEQ ID NO:43) and PEDV-S1-F (SEQ ID NO:44). The two resulting fragments were fused by OE-PCR using primers P290194A (SEQ ID NO:45) and P290194B (SEQ ID NO:43) to generate the PEDV spike BaculoDisplay (PEDVS BD) (SEQ ID NO:46) coding sequence containing a Kozak consensus sequence immediately 5′ of the start codon. The final coding sequence was flanked by BamHI and NotI restriction sites to facilitate cloning into baculovirus transfer plasmid pVL1393. Once completed, plasmid pVL1393-PEDVS BD was used with linearized BaculoGold baculovirus DNA to transfect Sf9 insect cells to produce recombinant baculovirus. See FIG. 6A. Schematic diagram of PEDV 2a BD Baculodisplay Baculovirus.

TABLE 6
PEDV Prototype Formulations and Controls

| Treatment | | Description |
|---|---|---|
| T01 | Negative Control | Saline (PBS 1×) |
| | (NC) | |
| T02 | Experimental | Porcine epidemic diarrhea virus at |
| | Vaccine (EV) | 6.93 log₁₀) TCID₅₀/mL PEDV |
| | | inactivated with BEI, adjuvanted |
| | | with 20% EMULSIGEN ® BCL. |
| T03 | EV | Recombinant baculovirus PEDV2a- |
| | | BD (PEDV Spike glycoprotein |
| | | displayed in the viral envelope.)* |
| T04 | EV | Recombinant baculovirus PEDV2a-BD |
| | | (PEDV Spike glycoprotein displayed |
| | | in the viral envelope.)* The clarified |
| | | inactivated material was concentrated |
| | | ~6X prior to formulation. |
| T05 | EV | Recombinant baculovirus PEDV2a-BD |
| | | (PEDV Spikeglycoprotein displayed |
| | | in the viral envelope.)* Recombinant |
| | | PEDV Spike-Display Baculovirus was |
| | | produced in insect cells with 10 μg/mL |
| | | trypsin added during infection. |
| T06 | Positive Control | iPED+ (Harris vaccine − |
| | (PC) | Conditionally Licensed) |

BEI = binary ethyleneimine
*The PEDV Spike signal sequence and C-terminal tail were replaced with the baculovirus gp64 equivalent. Recombinant PEDv Spike-Display Baculovirus was produced in insect cells. Infected cultures were harvested and clarified by centrifugation and 0.2-μm filtration. Clarified harvest material was inactivated with 5 mM BEI for 72 hours at 37° C. then clarified by centrifugation and 0.2-μm filtration.

### Serology:

Seroconversion post-vaccination (D28 & D35) occurred in 20% of pigs vaccinated with PEDv vaccine adjuvanted with 20% EMULSIGEN® BCL (T02; TABLE 7) and 60% of pigs vaccinated with trypsin-grown PEDV SPIKE-baculovirus (T05; TABLE 7). The geometric mean titer for seropositive pigs ≥1:20 for all treatment groups are presented below in TABLE 7. The frequency distribution of titers by treatment group of all pigs is presented in TABLE 8.

TABLE 7
Proportion of seropositive pigs and geometric mean
titer by group for pigs responding serologically

| Group | Pigs with ≥1:20 Response | Geometric Mean Titer |
|---|---|---|
| T01 | 0/10 (0%) | Not applicable |
| T02 | 4/20 (20%) | 1:30.7 |
| T03 | 0/9 (0%) | Not applicable |
| T04 | 0/10 (0%) | Not applicable |
| T05 | 6/10 (60%) | 1:35.5 |
| T06 | 7/10 (70%) | 1:41.9 |

TABLE 8
Frequency distribution of titers by group

| | | PEDv Neutralizing Antibodies* | | | | | | | | | | | | | | | |
|---|---|---|---|---|---|---|---|---|---|---|---|---|---|---|---|---|---|
| Group | n | <1:20 | | 1:20 | | 1:28 | | 1:40 | | 1:57 | | 1:80 | | 1:113 | | 1:160 | |
| T01 | 10 | 10 | (100%) | 0 | (0.0%) | 0 | (0.0%) | 0 | (0.0%) | 0 | (0.0%) | 0 | (0.0%) | 0 | (0.0%) | 0 | (0.0%) |
| T02 | 20 | 16 | (80%) | 1 | (5%) | 2 | (10%) | 0 | (0.0%) | 1 | (5%) | 0 | (0.0%) | 0 | (0.0%) | 0 | (0.0%) |
| T03 | 9 | 9 | (100%) | 0 | (0.0%) | 0 | (0.0%) | 0 | (0.0%) | 0 | (0.0%) | 0 | (0.0%) | 0 | (0.0%) | 0 | (0.0%) |
| T04 | 10 | 10 | (100%) | 0 | (0.0%) | 0 | (0.0%) | 0 | (0.0%) | 0 | (0.0%) | 0 | (0.0%) | 0 | (0.0%) | 0 | (0.0%) |
| T05 | 10 | 4 | (40%) | 2 | (20%) | 2 | (20%) | 1 | (10%) | 0 | (0.0%) | 0 | (0.0%) | 0 | (0.0%) | 1 | (10%) |
| T06 | 10 | 3 | (30%) | 2 | (20%) | 2 | (20%) | 0 | (0.0%) | 0 | (0.0%) | 2 | (20%) | 1 | (10%) | 0 | (0.0%) |

### Conclusion:

Seroconversion occurred in 20% of T02 pigs after two administrations of the experimental vaccine formulated with 6.93 log₁₀ TCID₅₀/mL PEDV inactivated with BEI and adjuvanted with 20% EMULSIGEN® BCL. Seroconversion occurred in 60% of T05 pigs after two administrations of the experimental recombinant trypsin grown-baculovirus vaccine formulated with PEDV Spike glycoprotein.

### Example 10

### Efficacy of PEDV 2a BD Baculovirus Vaccines

The primary objective of this study was to evaluate the efficacy of prototype PEDV baculovirus vaccines in a conventional sow model. Thirty-six sows were randomized into five groups. Sows were intramuscularly vaccinated at five and two weeks pre-farrow with an experimental PEDV vaccine, a placebo, a commercially available positive control or were left non-vaccinated (strict control). A brief description of the groups and vaccines used are listed in TABLE 9 below. Throughout gestation, farrowing and the challenge period, serum and fecal samples were collected from sows and pigs to monitor the presence of anti-PEDV-antibodies and PEDV RNA. Clinical signs were recorded once daily.

The primary outcome parameter of the study was pig mortality. Based on 69% pig mortality, the presence of clinical signs and identification of PEDV RNA in fecal material from pigs in the placebo group, the challenge model used for this study was considered valid. The use of two doses of the BaculoS-NT construct in sows was able to significantly reduce pig morality. In comparison, a PEDV vaccine currently on the market was also able to reduce pig morality; however, the reduction was not significant. As the BaculoS-NT prototype was not grown in the presence of trypsin and was adjuvanted with 12.5% EMULSIGEN® D, it is expected that this prototype would be the preferred format for production and have a 21 day withdrawal period.

A secondary outcome parameter of the study was the vaccine prototypes ability to prevent clinical signs in pigs. As greater than 93% of all pigs had pure liquid diarrhea for a minimum of two consecutive days, none of the vaccine prototypes or the commercially available product were able to prevent the onset of severe clinical signs. Similarly, none of the vaccines were able to reduce the duration of clinical signs in pigs.

TABLE 9
Study Design:

| | | | | Pigs |
|---|---|---|---|---|
| | N | N | | Challenged |
| Group | (sows) | (pigs) | Vaccine | (Y/N) |
| 1 | 8 | 82 | PEDV2a-BD Baculo | Y |
| | | | S-T (12.5% | |
| | | | EMULSIGEN ® D) | |
| 2 | 8 | 87 | PEDV2a-BD Baculo | Y |
| | | | S-NT (12.% | |
| | | | EMULSIGEN ® D) | |
| 5 | 8 | 81 | Placebo (1× PBS) | Y |
| 4 | 8 | 70 | Positive Control | Y |
| 5 | 4 | 46 | Strict Control | N |

Sows were intramuscularly vaccinated at five and two weeks pre-farrow with an experimental PEDV vaccine, a placebo, a commercially available positive control or were left non-vaccinated (strict control). During gestation, blood and a fecal sample were collected prior to each vaccination and assayed for evidence of seroconversion and shedding, respectively. Clinical observations were recorded on each sow daily.

### Pig Mortality:

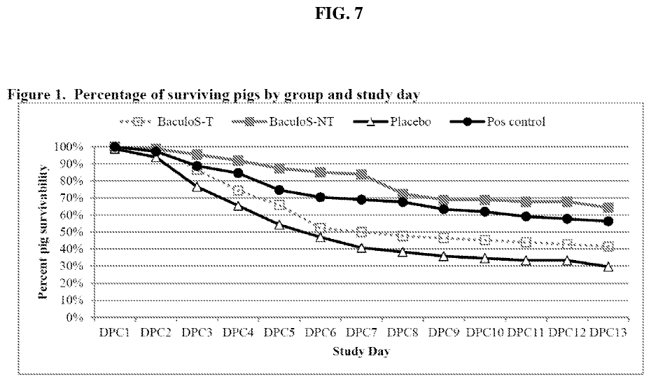

Pig mortality following challenge with a virulent PEDV isolate was the primary outcome parameter used to assess vaccine efficacy. A summary of mortality by group during the challenge period is presented in TABLE 10 below. Survivability by group and day is presented in FIG. 7. ‘Percentage of surviving pigs by group and day’). On D0, the 2 mL dose of vaccine was administered to healthy sows into the musculature of the right neck using an appropriately-sized, sterile needle and syringe. On D21, the process was identical with the exception that the injection was given on the left side of the neck.

Challenge material was virulent PEDV stock material (PEDV 1251-140-4; p5; L #2842-174; titer=2.02 x 10₅ TCID₅₀/mL) which was diluted to a titer of 1.0×10₃ in PEDV viral growth media (modified minimal essential media with HEPES, tryptose phosphate broth, and yeast extract). Similar to previous studies, the majority of mortalities were seen within eight days of exposure to challenge material.

Overall, all groups had similar trends with clinical signs present in the majority of pigs within 48 hours of infection. Clinical signs were present in greater than 50% of the pigs in all groups through DPC6. From DPC7 through DPC11 clinical signs resolved. By DPC13, no clinical signs were observed in any of the pigs.

### Analysis:

Biostatistics. All data were imported into SAS® version 9.4 for analysis. Data listings and summary statistics by treatment group, including frequency distributions, were generated. Analyses were conducted incorporating litter and housing effect as appropriate. Post-challenge mortality of pigs by group and litter was listed and descriptive statistics mean, median, minimum and maximum were used for summary by group. Mortality by group and litter was analyzed using a generalized linear model with Pearson's chi-square as over dispersion. Then, model-based Prevented Fraction and 95% Confidence Interval for each treatment group in comparison with the placebo group were estimated. A generalized linear model with Pearson's chi-square as over dispersion was fit to the mortality data one more time added with age, treated score and parity as fixed effects to look into if these factors have any effect on mortality. Study randomization data was listed. Fecal score for pigs was listed and summarized by group and day using frequency distribution. The summarization was done for prior to and during challenge separately. Score of clinical signs for sows (fecal, vomit, injection site lesion, Agalactia and treated) was listed and summarized separately for prior to and post farrow by group and day using frequency distribution. Pig weight data was listed and summarized using descriptive statistics median, minimum and maximum by litter, group and day. Average Daily Weight Gain (ADWG) was analyzed by a mixed model with group and weight at farrowing as fixed effects; litter and room for housing as random effects. Least square means and contrast of them between groups were estimated; the corresponding standard errors and 95% confidence intervals were estimated. Necropsy data for pigs, including pathology data for organ and content for cecum, colon, small intestine, and lung lesions, and stomach record, was listed and summarized using counts for each variable by group separated by mortality. Whether there existed any lesion was also summarized using counts and percentage by group. Lab data, including PED virus serum antibody titer (FFN) for sows, serum IgA whole cell ELISA for pigs and sows separately, IgA and IgG baculos ELISA for sows, IgA and IgG IDEXX ELISA and PCR for sows and pigs, were first listed. Next, descriptive statistics mean, minimum, maximum and standard deviation by group and day were calculated using either geometric means (FFN) (although for FFN, standard deviation was not provided) or means (ELISA, PCR). Next, least square means, standard errors and 95% confidence intervals by group and day were estimated using a mixed model with litter and room for housing as random effects. Contrast of least square means between pairs of groups and the corresponding 95% confidence intervals were estimated.

TABLE 10
Pig Mortality by Group

| | | % Pig Mortality | | | | |
|---|---|---|---|---|---|---|
| | Descrip- | | | Mini- | Maxi- | |
| Group | tion | Mean | Median | mum | mum | PF |
| 1 | BaculoS- | 59.03 | 59.60 | 8.33 | 100.00 | 0.17 (−0.244, |
| | T | | | | | 0.444) |
| 2 | BaculoS- | 36.31 | 44.16 | 0.00 | 58.33 | 0.48 (0.103, |
| | NT | | | | | 0.695) |
| 3 | Placebo | 68.95 | 69.32 | 45.45 | 91.67 | Not applicable |
| 4 | Positive | 42.83 | 32.47 | 9.09 | 100.00 | 0.39 (−0.037, |
| | control | | | | | 0.642) |

The primary outcome parameter of the study was pig mortality. Based on this parameter, use of two doses of the BaculoS-NT construct in sows was able to significantly reduce pig morality (PF=0.48). In comparison, a PEDV vaccine currently on the market was also able to reduce pig morality; however, the reduction was not significant (Group 4). As the BaculoS-NT prototype was not grown in the presence of trypsin and was adjuvanted with 12.5% EMULSIGEN® D, it is expected that this prototype would be the preferred format for production and have a 21 day withdrawal period.

Secondary outcomes parameters of the study were the vaccine prototypes ability to prevent clinical signs in pigs and sows. As greater than 93% of all pigs had pure liquid diarrhea for a minimum of two consecutive days, none of the vaccine prototypes or the commercially available product were able to prevent the onset of severe clinical signs. Similarly, none of the vaccines were able to reduce the duration of clinical signs in pigs. Following resolution of clinical signs, PEDV RNA was identified in 12-33% of animals at DPC14. This suggests that vaccination did not protect against colonization. In 1-3 sows per group, severe diarrhea was observed for at least two consecutive days. This indicates that the vaccines were not able to fully protect sows against exposure to high amounts of virus shed by challenged pigs. In addition, the majority of sows (14/16) had detectable amounts of virus present in mesenteric lymph nodes indicating vaccination did not prevent colonization.

The majority of vaccinated sows had a detectable IgG (and FFN) response following two doses of vaccine; titers were higher in comparison to placebo-vaccinated controls (data not shown). As IgG ELISA and FFN results are very similar, it is likely that the FFN assay is mainly detecting IgG. While IgG can neutralize virus, it is unlikely to be the sole determinate of PEDV vaccine efficacy as clear differences between groups were not apparent.

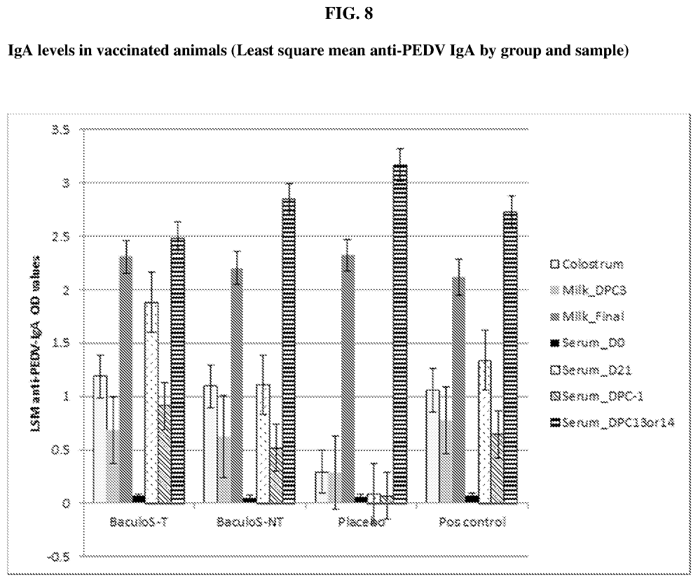

Based on IgA levels as measured IgA ELISA (IDEXX), naïve and vaccinated sows had similar serum and milk IgA titers by DPC13/14. However, increased IgA titers in serum, colostrum and milk collected at DPC3 were noted in vaccinated animals. As surviving pigs from vaccinated sows also had the highest IgA titers, this data supports previously published literature emphasizing the importance of IgA in enteric infections. (See FIG. 8).

### Example 11

### Preparation of PEDV2b Baculovirus Vaccines

The spike gene from porcine epidemic diarrhea virus (PEDV) 2b was prepared in two overlapping fragments (N-term and C-term). The N-terminal fragment was amplified from synthetic DNA containing a fusion of the Autographa californica nucleopolyhedrovirus (AcNPV) gp64 signal sequence with amino acids 22-400 of PEDV 2b spike using primers P290194A (SEQ ID NO:48) and P3183131B (SEQ ID NO:49). The C-term fragment was amplified from pVL1393-PEDVS BD plasmid DNA which already contained the AcNPV gp64 C-terminal tail coding sequence using primers P3183131A (SEQ ID NO:50) and P290194B (SEQ ID NO:51). The two resulting fragments were fused by OE-PCR using primers P290194A (SEQ ID NO:48) and P290194B (SEQ ID NO:51) to generate the PEDV 2b spike BaculoDisplay (PEDVS 2b BD) coding sequence (SEQ ID NO:52) containing a Kozak's consensus sequence immediately 5′ of the start codon. The final coding sequence was flanked by BamHI and NotI restriction sites to facilitate cloning into baculovirus transfer plasmid pVL1393. Once completed, plasmid pVL1393-PEDVS 2b BD was used with linearized BaculoGold baculovirus DNA to transfect Sf9 insect cells to produce recombinant baculovirus. See FIG. 6B. Schematic diagram of PEDV 2b BD Baculodisplay Baculovirus-Protein map.

### Example 12: PEDV and PDCoV Efficacy Studies

The below study was designed to assess the efficacy of a killed PEDV vaccine and/or killed PDCoV vaccine or other prototype vaccines in pigs. The primary outcome parameter is piglet mortality following challenge with a porcine epidemic diarrhea virus (PEDV) and/or PDCoV. The secondary outcome parameter was dam serology. Other parameters measured included: clinical signs (including ISL) in sows following vaccination; viral shedding in sows following vaccination (via qRT-PCR); clinical signs in piglets; and PEDV and/or PDCoV serology in piglets

### PEDV Study:

At four and two weeks pre-farrow (D0 and D14), each gestating dam was administered 2 mL of one of the following treatments by three routes (intramuscular, intranasal and oral): T01 (negative control, NC) phosphate buffered saline; T02 (BEI-VH) adjuvanted with 20% EMULSIGEN® BCL; T03 (strict control, SC) served as non-vaccinated/non-challenged control. Eight animals were used per group, excluding T06 which had four animals. On D35 or D36, pigs were challenged orally with 1 mL of 2.0 log10 TCID50/mL PEDV viral harvest. Clinical signs (vomiting and diarrhea) in dams and pigs were observed daily during the challenge phase. Serum was collected from dams at four- and two-weeks pre-farrow (D0 and D14), the day prior to piglet challenge (D34 or D35) and the day of off-test (D57).

On D0 and D14, the PEDV prototype immunogenic compositions were administered to the sows. At each vaccination, the sows received a total of 6 mL of material where 2 mL were administered by intramuscular, intranasal and oral route. For the intramuscular administration route, a 2 mL injection was given into the musculature of the neck below the ear. The side of the neck for administration was alternated for the initial and booster vaccination. For the oral administration route, 2 ml were delivered over the caudal oropharynx using an 8Fr polypropylene catheter (2.7 mm diameter by 254 mm length) attached to a syringe. For the intranasal route, 1 ml was injected into each nare using a 4.5 inch catheter attached to a syringe.

TABLE 11
PEDV Experimental Immunogenic Compositions and Control Product

| Treatment | Group | Serial # | Description |
|---|---|---|---|
| T01 | NC | 2842-182-D | 1X Phosphate Buffered Saline; Gibco |
| | | | catalog no. 10010-023; Lot no. 1510272 |
| T02 | BEI-VH | 2842-182-E | KV-1251-125-10-OK, 0.2 μm filtered, passage MSV + |
| | | | 5, 6.04 log TCID₅₀/mL. Viral harvest was inactivated |
| | | | with 5 mM BEI for 72 hr at 37° C. |
| | | | For formulation, EMULSIGEN ® BCL |
| | | | (MVP lot no. 17006, manufacture date Feb. 11, 2011) |
| | | | was added at a 20% inclusion rate. |

TABLE 12
PEDV Challenge Material

| | |
|---|---|
| PEDV Challenge Strain: | Isolate id. 1251-140-4; passage 5 |
| Challenge preparation: | Propagated in Vero cells |
| Dose of Challenge material: | 1 mL at 2.0 log₁₀ TCID₅₀/mL |
| Testing of Challenge | Challenge virus was titrated prior |
| Material: | to administration on 2013 EU |
| | Vero cells (5.03 TCID50/ml) |
| | and diluted to 2 log TCID₅₀/mL. |
| Method of Administration: | Oral administration (by syringe) |
| | with pigs manually restrained. |

### Vaccine Efficacy:

Pig mortality: Pig mortality following challenge with a virulent PEDV isolate was the primary outcome parameter used to assess vaccine efficacy. A summary of mortality by group during the challenge period is listed below. With 55% mortality and all litters affected in T01 (NC), the challenge was considered sufficiently virulent. In comparison to T01 (NC), T02 (BEI-VH) demonstrated a numerical reduction in pig mortality with a PF (95% CI) of 0.20 (−0.550, 0.586). The reduction was not statistically significant as the 95% CI (−0.550, 0.586) included zero. (See TABLE 13).

Extra-binomial variation was evident in this study, resulting in a wide confidence interval for T02 (BEI-VH) PF when utilizing the underlying binomial distribution. Mortality varied greatly among litters within a group, including ranging from 0% to 100% for T02 (BEI-VH).

An intestinal sample or intestinal content was taken at the time necropsy and tested by qRT-PCR to detect PEDV antigen. Of samples tested from animals during the time of peak mortality, PEDV was detected in 55.5% of samples.

TABLE 13

| | Propor- | | | 95% | | |
|---|---|---|---|---|---|---|
| | tion | Stand- | Pre- | Confi- | Median | |
| | Mortality | ard | vented | dence | Mor- | Minimum %/ |
| Group | Estimate | Error | Fraction* | interval | tality | Maximum % |
| NC | 0.55 | 0.11 | — | — | 52.78 | 12.50/ |
| | | | | | | 100.00 |
| BEI- | 0.44 | 0.10 | 0.20 | (−0.550, | 34.29 | 0.00/ |
| VH | | | | 0.586) | | 100.00 |

*Based on T01 (NC) proportion affected.
**NC = Not Calculated. Confidence Interval possible for T02 (BEI-VH) based on study design

### Sow Serology:

Fluorescent Focus Neutralizing (FFN) assay: The FFN assay was used to assess the dam virus neutralizing response following vaccination and challenge. Geometric mean titers listed by group are presented below for days on which blood was collected from sows.

Following two doses of vaccine, 2/8 (25%) of sows in T02 (BEI-VH) had detectable levels of neutralizing antibody. Detectable levels of neutralizing antibody were not observed in any of the other groups.

Following lateral exposure to PEDV, all sows in exposed treatment groups had detectable levels of neutralizing antibody. Animals in T03 (SC) group remained seronegative throughout the trial. The geometric mean titer on D57 (approximately 21 days post-exposure) indicated that vaccination resulted in numerically higher titers in comparison to T01 (NC). Sows in T02 (BEI-VH) group had a GMT of 613, which is an approximately three-fold higher titer in comparison to the GMT of 200 for sows in T01 (NC) (p=0.005). As multiple samples in T02 (BEI-VH) group had detectable neutralizing antibodies at the highest dilution tested (1:640), these results likely represent a conservative estimate of the differences between groups.

TABLE 14
Sow Serology

| | | Geometric Mean Titer* | | | |
|---|---|---|---|---|---|
| | | Study Day** | | | |
| | | | | D34 or | |
| Treatment | Group | D0 | D14 | D57 | D35 |
| T01 | NC | <20 | <20 | <20 | 200 |
| T02 | BEI-VH | <20 | <20 | 15 | 613 |
| T03 | SC | <20 | <20 | <20 | <20 |

Where all values were <20, geometric mean titer is presented as <20. Otherwise, values of <20 were set to 10 for GMT calculation
**D57 GMT for T01 (NC) and T02 (BEI-VH) are back-transformed Least-Squares Means

### S1-Based ELISA Data:

An S1-based ELISA was used to assess the dam's response to the PEDV-spike protein following vaccination and challenge. Assay results for colostrum, milk and serum are listed by group for days on which samples were collected.

At the time of pig challenge, sows in T02 (BEI-VH) had significantly higher geometric mean titer in serum as compared to sows in T01 (NC) (p=0.0005). Following exposure to PEDV, a larger significant difference was noted between the two groups (p<0.0001).

Significant differences in geometric mean titers of anti-PEDV IgA in colostrum and in milk were not observed between T02 (BEI-VH) and T04 (NC).

TABLE 15

| | | Geometric Mean Titer* | | | |
|---|---|---|---|---|---|
| | | Study Day | | | |
| | | D27 | | | |
| | | through | D34 or | | |
| | | D32: | D35: | D57: | D57: |
| Treatment | Group | Colostrum | Serum | Serum | Milk |
| T01 | NC | 0.186 | 0.098 | 0.504 | 0.220 |
| T02 | BEI-VH | 0.139 | 0.256 | 1.499 | 0.244 |
| T03 | SC | 0.134 | 0.125 | 0.164 | 0.088 |

*GMT for T01 (NC) and T02 (BEI-VH) are back-transformed Least-Squares Means

### Pig Serology:

Serum was collected at the time of necropsy from pigs to evaluate the presence of neutralizing antibodies. TABLE 16 below presents the geometric mean FFN titers of positive pigs by group. TABLE 16 also includes the frequency of detection expressed as the number of pigs with a GMT greater than or equal to 20 over the number of animals tested. Testing was performed on all available samples. Samples from numerous pigs were unable to be obtained due to the time difference between death and necropsy.

Descriptive statistics for FFN titers by mortality status (Died: Yes/No) and group (Overall) are listed below. Overall, a similar proportion of pigs in the vaccinated groups seroconverted (or had maternal antibodies) regardless of time of necropsy. However, in T01(NC), a higher percentage of pigs that died prior to off test had titers (88%) in comparison to pigs that lived for the duration of the study (43%).

When looking at the overall pig titers by group, the proportion mortality estimate was inversely related to the overall group FFN percentage for T02 (BEI-VH).

TABLE 16

| | | | | Proportion |
|---|---|---|---|---|
| | Pigs | Pigs | | Mortality |
| Group | (Died = yes) | (Died = no) | Overall | Estimate |
| NC | 55 | 33 | 63 | 0.55 |
| | (28/32; 88%) | (9/21; 43%) | (37/53; 59%) | |
| BEI-VH | 44 | 50 | 64 | 0.44 |
| | (23/42; 55%) | (9/16; 56%) | (32/58; 55%) | |

* GMT (no. animals titer ≥20/total pigs tested; percentage); note that serum was not obtained from all pigs.

### Clinical Observations Following Challenge:

Pig fecal scores: Descriptive statistics for the duration of abnormal fecal observations in pigs, by group and mortality status (Died: Yes/No), are listed below. Overall, the median duration of abnormal fecal scores in pigs with the same mortality status was similar among groups. In animals that died or were euthanized, there was a numerically shorter median duration of abnormal fecal scores. This trend was most evident in T01 (NC) pigs and is likely secondary to the fact that the majority of these animals died within the first week following challenge.

TABLE 17
Duration (days) abnormal fecal score

| | | # | | | | |
|---|---|---|---|---|---|---|
| Died | Group | pigs | Median | Minimum | Maximum | Std Dev |
| No | NC | 32 | 5.5 | 3.5 | 7.0 | 0.8 |
| | BEI-VH | 42 | 6.0 | 4.5 | 8.5 | 0.8 |
| Yes | NC | 39 | 2.3 | 0.5 | 6.0 | 1.3 |
| | BEI-VH | 33 | 4.3 | 2.0 | 6.0 | 1.4 |

The severity of fecal scores in pigs is summarized in the frequency TABLE 18 below. In all treatment groups a high portion of pigs (>91%) presented with a fecal score of 2 during at least one observation following challenge.

TABLE 18
Maximum Fecal Score

| Group | 0 | 1 | 2 | Total |
|---|---|---|---|---|
| NC | 1 | 5 | 65 | 71 |
| | 1.41 | 7.04 | 91.55 | |
| BEI-VH | 1 | 0 | 74 | 75 |
| | 1.33 | 0.00 | 98.67 | |
| Total | 2 | 5 | 139 | 146 |

### Conclusions:

A 20% reduction in pig mortality was observed in T02 (BEI-VH) as compared to T01 (NC) group. Three routes of administration were attempted in this study. Although 3 routes were used, there is no expectation that routes other than IM contributed to the efficacy of T02 (BEI-VH) based on the adjuvant and vaccine formulation. Overall the inactivated PEDV adjuvanted with 20% EMULSIGEN®-BCL vaccine with a minimum pre-inactivation titer of 6.04 log TCID₅₀/ml appears to induce better immune responses in the piglets and sows. The preferred vaccination schedule is IM route of administration for piglets 3 weeks of age or older, three 2 ml doses at 2-week intervals. Clinical signs in sows following vaccination were not observed in T02 (BEI-VH) and were limited in the other treatment groups. The use of vaccination did not appear to affect the percentage of pigs born live (data not shown).

Dam serology was evaluated as a secondary parameter by two separate assays (focus fluorescent neutralization, S1-based ELISA). Both assays indicated a significant increase in titer in T02 (BEI-VH) following vaccination and exposure as compared to T01 (NC). Due to known limitations of the FFN assay, samples were also tested by an S1-based ELISA. This ELISA was chosen as the S1 domain of the spike protein is expected to contain neutralizing epitopes.

Following lateral exposure to PEDV, all animals in exposed treatment groups had detectable levels of neutralizing antibody. Sows in T02 (BEI-VH) had approximately three-fold higher titers in comparison to the T01 (NC) animals. This is evidence that use of the vaccine stimulated an initial primary response and resulted in a greater secondary response following exposure to the challenge virus. As multiple samples in T02 (BEI-VH) group had detectable neutralizing antibodies at the highest dilution tested (1:640), these results likely represent a conservative estimate of the differences between groups.

TABLE 19
Pig mortality and sow serological data are summarized below.

| | | | IgG | | |
|---|---|---|---|---|---|
| | | FFN | ELISA | | |
| | | (Sow | (Sow | Pig | Prevented |
| | | serum, | serum, | Mortality | Fraction (pig |
| Treatment | Group | D21) | D21) | (%) | mortality) |
| T01 | NC | 200 | 0.504 | 55% | — |
| T02 | BEI-VH | 613 | 1.499 | 44% | 0.20 |
| T03 | SC | <20 | 0.164 | NA | NA |

### PDCoV Study

The objective of this study was to determine the efficacy of prototype PDCoV vaccines in the host animal. Based on below preliminary study to develop challenge material and a disease model of PDCoV, unvaccinated piglets exhibited weight loss and diarrhea within a week of challenge and recovered. No mortality was observed.

TABLE 20
Porcine Deltacoronavirus (PDCoV) Challenge Material:

| | No of | No of | Challenge | | |
|---|---|---|---|---|---|
| Group | Sows | Piglets | virus/treatment | Titer | Dose |
| 1 | 1 | 6 | NVSL-PDCoV P12 | 6.1 log₁₀ | 1 mL/oral |
| | | | | TCID₅₀/ | |
| | | | | piglet | |
| 2 | 1 | 5 | Placebo (PDCoV | N/A | 1 mL/oral |
| | | | maintenance media) | | |
| 3 | 1 | 12 | BI PDCoV 5.0327 | 4.6 log₁₀ | 1 mL/oral |
| | | | | TCID₅₀/ | |
| | | | | piglet | |
| 4 | 1 | 12 | BI PDCoV 2.0307 | 3.9 log₁₀ | 1 mL/oral |
| | | | | TCID₅₀/ | |
| | | | | piglet | |

To establish a challenge strain for porcine Deltacoronavirus (PDCoV), two internal challenge isolates (Groups 3 and 4) were grown on BI-ST cells and compared to the NVSL PDCoV isolate (Illinois 2014). Isolates in groups 3 and 4 were selected based on the mutations they possessed and their genetic distance from the NVSL (Illinois 2014) isolate. The group 3 virus (BI PDCoV-5.0327) had a 3 amino acid deletion in the spike glycoprotein and two changes in the ORFIAB gene in comparison to the NSVL PDCoV isolate. The group 4 virus (BIPDCoV 2.0307) had 4 amino acid differences in the spike glycoprotein and 3 differences in the ORF1AB gene in comparison to the NVSL PDCoV.

Challenged piglets were scored for diarrhea where 0=normal, 1=abnormal (not pure liquid) or 2=severe diarrhea (pure liquid feces) post challenge. No diarrhea was observed in the negative control group. Additionally, the group challenged with NVSL PDCoV did not show any clinical signs, weight loss, vomiting or diarrhea post challenge. Piglets in groups 3 and 4 exhibited severe clinical signs with a 100% of them experiencing severe diarrhea (score of 2) accompanied by vomiting and weight loss. In addition, the sows in groups 3 and 4 suffered from diarrhea (D6-D8 post challenge) and were treated. Previous published studies described prolonged infection (and shedding of virus) among piglets infected with PDCoV. Their observations are in agreement with our results.

Animals in the NVSL PDCoV and negative control groups gained weight post challenge. In comparison, animals challenged with the two challenge isolates BI PDCoV-5.0327 (Group 3) and BI PDCoV 2.0307 (Group 4) exhibited less robust weight gain during the same period as compared to the control group.

### Efficacy Study:

TABLE 21
PDCoV Study design (vaccine groups, dose and routes)

| | | | | Dose |
|---|---|---|---|---|
| | | | | (5 and |
| | N | Vaccine | Vaccine | 2 weeks |
| Group | sows | primary | Route | pre-farrow) |
| 1 | 4 | BEI inactivated PDCoV | IM | 2 mL |
| 2 | 4 | BEI inactivated Baculo- | IM | 2 mL |
| | | spike-FcR | | |
| 3 | 4 | NVSL-PDCoV (Live | Oral | 2 mL |
| | | attenuated) | | |
| 4 | 4 | Placebo (Virus maint. | IM | 2 mL |
| | | media) | | |

For the efficacy study, four groups of sows are vaccinated with each vaccine formulation as detailed in TABLE 21 by the appropriate route at 5 and 2 weeks pre-farrow. Serum is collected from the sows prior to administration of the treatment at each vaccination for seroconversion assays. Fecal samples are collected from sows at each vaccination and assayed for evidence of vaccine shedding.

Piglets from the immunized sows are enrolled into the trial at the time of farrowing. Piglets are inoculated at approximately five to seven days of age, with 1.0 mL of challenge material using the oral route.

Both piglets and sows are monitored for clinical signs once daily from days post challenge (DPC) 0 through DPC14. Post challenge fecal and serum samples are taken from all piglets and sows. Fecal samples are tested for the presence of PDCoV RNA and serum is tested for the presence of anti-PDCoV antibodies. Colostrum is collected at or near the time of farrowing from all sows. Milk is collected from all sows at DPC3 and at one point between DPC10 through DPC14. Colostrum and milk are tested for the presence of anti-PDCoV antibodies. Piglets' weight is monitored on DPC 0, 7 and 21 with terminal serum collection on DPC21.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

The following references, to the extent that they provide exemplary procedural or other details supplementary to those set forth herein, are specifically incorporated herein by reference.

### REFERENCES

1. Bridgen A, Duarte M, Tobler K, Laude H, Ackermann M. 1993. Sequence determination of the nucleocapsid protein gene of the porcine epidemic diarrhoea virus confirms that this virus is a coronavirus related to human coronavirus 229E and porcine transmissible gastroenteritis virus. J. Gen. Virol. 74 (Pt 9):1795-1804.
2. Duarte M, Gelfi J, Lambert P, Rasschaert D, Laude H. 1993. Genome organization of porcine epidemic diarrhoea virus. Adv. Exp. Med. Biol. 342:55-60
3. Tobler K, Bridgen A, Ackermann M. 1993. Sequence analysis of the nucleocapsid protein gene of porcine epidemic diarrhoea virus. Adv. Exp. Med. Biol. 342:49-54.
4. Oldham J. 1972. Letter to the editor. Pig Farming 1972 (October suppl): 72-73.
5. Pensaert MB, de Bouck P. 1978. A new coronavirus-like particle associated with diarrhea in swine. Arch. Virol. 58:243-247.
6. Chen JF, Sun DB, Wang CB, Shi HY, Cui XC, Liu SW, Qiu HJ, Feng L. 2008. Molecular characterization and phylogenetic analysis of membrane protein genes of porcine epidemic diarrhea virus isolates in China. Virus Genes 36:355-364.
7. Nagy B, Nagy G, Meder M, Mocsári E. 1996. Enterotoxigenic Escherichia coli, rotavirus, porcine epidemic diarrhoea virus, adenovirus and calici-like virus in porcine postweaning diarrhoea in Hungary Acta Vet. Hung. 44:9-19.
8. Martelli P, Lavazza A, Nigrelli AD, Merialdi G, Alborali LG, Pensaert MB. 2008. Epidemic of diarrhoea caused by porcine epidemic diarrhoea virus in Italy. Vet. Rec. 162:307-310.
9. Takahashi K, Okada K, Ohshima K. 1983. An outbreak of swine diarrhea of a new-type associated with coronavirus-like particles in Japan. Nippon Juigaku Zasshi 45:829-832.
10. Chae C, Kim O, Choi C, Min K, Cho WS, Kim J, Tai JH. 2000. Prevalence of porcine epidemic diarrhoea virus and transmissible gastroenteritis virus infection in Korean pigs. Vet. Rec. 147:606-608
11. Puranaveja S, Poolperm P, Lertwatcharasarakul P, Kesdaengsakonwut S, Boonsoongnern A, Urairong K, Kitikoon P, Choojai P, Kedkovid R, Teankum K, Thanawongnuwech R. 2009. Chinese-like strain of porcine epidemic diarrhea virus, Thailand. Emerg. Infect. Dis. 15:1112-1115.
12. Stevenson GW, Hoang H, Schwartz KJ, Burrough ER, Sun D, Madson D, Cooper VL, Pillatzki A, Gauger P, Schmitt BJ, Koster LG, Killian ML, Yoon KJ. 2013. Emergence of porcine epidemic diarrhea virus in the United States: clinical signs, lesions, and viral genomic sequences. J. Vet. Diagn. Invest.25:649-654.
13. Kim SH, Kim IJ, Pyo HM, Tark DS, Song JY, Hyun BH. 2007. Multiplex real-time RT-PCR for the simultaneous detection and quantification of transmissible gastroenteritis virus and porcine epidemic diarrhea virus. J. Virol. Methods 146:172-177.
14. Hofmann M, Wyler R. 1988. Propagation of the virus of porcine epidemic diarrhea in cell culture. J. Clin. Microbiol. 26:2235-2239.
15. Marthaler D, Jiang Y, Otterson T, Goyal S, Rossow K, Collins J. 2013. Complete genome sequence of porcine epidemic diarrhea virus strain USA/Colorado/2013 from the United States. Genome Announc.1(4):e00555-13.10.1128/genomeA.00555-13
16. Song D, Park B. 2012. Porcine epidemic diarrhoea virus: a comprehensive review of molecular epidemiology, diagnosis, and vaccines. Virus Genes 44:167-175.
17. Huang YW, Dickerman AW, Pi\ddot{n}eyro P, Li L, Fang L, Kiehne R, Opriessnig T, Meng XJ. 2013.Origin, evolution, and genotyping of emergent porcine epidemic diarrhea virus strains in the United States. mBio 4(5):e00737-13.
18. Bi J, Zeng S, Xiao S, Chen H, Fang L. 2012. Complete genome sequence of porcine epidemic diarrhea virus strain AJ1102 isolated from a suckling piglet with acute diarrhea in China. J. Virol. 86:10910-10911.
19. Chen J, Wang C, Shi H, Qiu HJ, Liu S, Shi D, Zhang X, Feng L. 2011. Complete genome sequence of a Chinese virulent porcine epidemic diarrhea virus strain. J. Virol. 85:11538-11539.
20. Chen J, Liu X, Shi D, Shi H, Zhang X, Feng L. 2012. Complete genome sequence of a porcine epidemic diarrhea virus variant. J. Virol. 86:3408.10.1128/JVI.07150-11
21. Fan H, Zhang J, Ye Y, Tong T, Xie K, Liao M. 2012. Complete genome sequence of a novel porcine epidemic diarrhea virus in south China. J. Virol. 86:10248-10249.
22. Gao Y, Kou Q, Ge X, Zhou L, Guo X, Yang H. 2013. Phylogenetic analysis of porcine epidemic diarrhea virus field strains prevailing recently in China. Arch. Virol. 158:711-715.
23. Li B, Liu H, He K, Guo R, Ni Y, Du L, Wen L, Zhang X, Yu Z, Zhou J, Mao A, Lv L, Hu Y, Yu Y, Zhu H, Wang X. 2013. Complete genome sequence of a recombinant porcine epidemic diarrhea virus strain from eastern China. Genome Announc. 1(2):e00105-13.10.1128/genomeA.00105-13
24. Luo Y, Zhang J, Deng X, Ye Y, Liao M, Fan H. 2012. Complete genome sequence of a highly prevalent isolate of porcine epidemic diarrhea virus in south China. J. Virol. 86:9551-9551.
25. Wang XM, Niu BB, Yan H, Gao DS, Huo JY, Chen L, Chang HT, Wang CQ, Zhao J. 2013.Complete genome sequence of a variant porcine epidemic diarrhea virus strain isolated in central China.Genome Announc. 1(1): e00243-12.10.1128/genomeA.00243-12
26. Wei ZY, Lu WH, Li ZL, Mo JY, Zeng XD, Zeng ZL, Sun BL, Chen F, Xie QM, Bee YZ, Ma J-Y. 2012.Complete genome sequence of novel porcine epidemic diarrhea virus strain GD-1 in China. J. Virol.86:13824-13825.
27. Zhao M, Sun Z, Zhang Y, Wang G, Wang H, Yang F, Tian F, Jiang S. 2012. Complete genome sequence of a Vero cell-adapted isolate of porcine epidemic diarrhea virus in eastern China. J. Virol.86:13858-13859.
28. S.H. Chang, J.L. Bae, T.J. Kang, J. Kim, G.H. Chung, C.W. Lim, H. Laude, M.S. Yang, Y.S. Jang. 2002. Identification of the epitope region capable of inducing neutralizing antibodies against the porcine epidemic diarrhea virus. Mol. Cells 14, 295-299.
29. D.J. Cruz, C.J. Kim, H.J. Shin. 2008. The GPRLQPY motif (SEQ ID NO: 20) located at the carboxy-terminal of the spike protein induces antibodies that neutralize Porcine epidemic diarrhea virus. Virus Res. 132, 192-196.
30. M. Godet, J. Grosclaude, B. Delmas, H. Laude. 1994. Major receptor-binding and neutralization determinants are located within the same domain of the transmissible gastroenteritis virus (coronavirus) spike protein. J. Virol. 68, 8008-8016.
31. M.W. Jackwood, D.A. Hilt, S.A. Callison, C.W. Lee, H. Plaza, E. Wade. 2001. Spike glycoprotein cleavage recognition site analysis of infectious bronchitis virus. Avian Dis. 45, 366-372.
32. L.S. Sturman, K.V. Holmes. 1984 Proteolytic cleavage of peplomeric glycoprotein E2 of MHV yields two 90K subunits and activates cell fusion. Adv. Exp. Med. Biol. 173, 25-35.
33. D. Sun, L. Feng, H. Shi, J. Chen, X. Cui, H. Chen, S. Liu, Y. Tong, Y. Wang, G. Tong. 2008. Identification of two novel B cell epitopes on porcine epidemic diarrhea virus spike protein. Vet. Microbiol. 131, 73-81.
34. S.J. Park, H.J. Moon, J.S. Yang, C.S. Lee, D.S. Song, B.K. Kang, B.K. Park. 2007. Sequence analysis of the partial spike glycoprotein gene of porcine epidemic diarrhea viruses isolated in Korea. Virus Genes 35, 321-332.
35. L.J. Saif. 1993. Coronavirus immunogens. Vet. Microbiol. 285-297.
36. S.J. Park, H.K. Kim, D.S. Song, H.J. Moon, B.K. Park. 2011 Molecular characterization and phylogenetic analysis of porcine epidemic diarrhea virus (PEDV) field isolates in Korea. Arch. Virol. 156, 577-585.
37. D.S. Song, J.S. Yang, J.S. Oh, J.H. Han, B.K. Park. Differentiation of a Vero cell adapted porcine epidemic diarrhea virus from Korean field strains by restriction fragment length polymorphism analysis of ORF 3. 2003. Vaccine 21, 1833-1842.
38. D.S. Song, J.S., B.K. Park. 2012 Porcine epidemic diarrhoea virus: a comprehensive review of molecular epidemiology, diagnosis, and vaccines. Virus Genes 44, 167-175.
39. J.F. Chen, D.B. Sun, C.B. Wang, H.Y. Shi, X.C. Cui, S.W. Liu, H.J. Qiu, L. Feng. 2008. Molecular characterization and phylogenetic analysis of membrane protein genes of porcine epidemic diarrhea virus isolates in China. Virus Genes 36, 355-364.
40. L. Yuan, S.Y. Kang, L.A. Ward, T.L. To, L.J. Saif. 1998 Antibody-secreting cell responses and protective immunity assessed in gnotobiotic pigs inoculated orally or intramuscularly with inactivated human rotavirus. J. Virol. 72, 330-338.
41. C.H. Kweon, B.J. Kwon, J.G. Lee, G.O. Kwon, Y.B. Kang. 1999. Derivation of attenuated porcine epidemic diarrhea virus (PEDV) as vaccine candidate. Vaccine 17, 2546-2553.
42. Y. Usami, O. Yamaguchi, K. Kumanomido, Y. Matsumura. 1998. Antibody response of pregnant sows to porcine epidemic diarrhea virus live vaccine and maternally-derived antibodies of the piglets. J. Jpn. Vet. Med. Assoc. 51, 652-655.
43. L.A. Ward, L. Yuan, B.I. Rosen, T.L. To, L.J. Saif. 1996. Development of mucosal and systemic lymphoproliferative responses and protective immunity to human group A rotaviruses in a gnotobiotic pig model. Clin. Diagn. Lab. Immunol. 3, 342-350.
44. A. Pijpers, A.P. van Nieuwstadt, C. Terpstra, J.H. Verheijden. 1993. Porcine epidemic diarrhoea virus as a cause of persistent diarrhoea in a herd of breeding and finishing pigs. Vet. Rec. 132, 129-131.
45. T. Sato, Takeyama, N., Katsumata, A., Tuchiya, K., Kodama, T., Kusanagi, K. 2011. Mutations in the spike gene of porcine epidemic diarrhea virus associated with growth adaptation in vitro and attenuation of virulence in vivo. Virus Genes, 43, 1, 72.
46. Park, S.J., Kim, H.K., Song, D.S., An, D.J. and Park, B.K. 2012. Complete genome sequences of a Korean virulent porcine epidemic diarrhea virus and its attenuated counterpart J. Virol. 86 (10), 5964.
47. Kusanagi K, Kuwahara H, Katoh T, Nunoya T, Ishikawa Y, Samejima T, Tajima M. 1992. Isolation and serial propagation of porcine epidemic diarrhea virus in cell cultures and partial characterization of the isolate. J Vet Med Sci. 1992 Apr;54(2):313-8.
48. Hofmann M, Wyler R. 1988. Propagation of the virus of porcine epidemic diarrhea in cell culture. J Clin Microbiol. Nov;26(11):2235-9.49. de Arriba ML, Carvajal A, Pozo J, Rubio P. 2002. Mucosal and Systemic Isotype-specific Antibody Responses and Protection in Conventional Pigs Exposed to Virulent or Attenuated Porcine Epidemic Diarrhoea Virus. Vet Immunol Immunopathol. 85(1-2): p. 85-97.
50. Woo, P.C., et al., Discovery of seven novel Mammalian and avian coronaviruses in the genus Deltacoronavirus supports bat coronaviruses as the gene source of Alphacoronavirus and Betacoronavirus and avian coronaviruses as the gene source of Gammacoronavirus and Deltacoronavirus. J Virol, 2012. 86(7): p. 3995-4008.
51. Hu, H., et al., Isolation and Characterization of Porcine Deltacoronavirus from Pigs with Diarrhea in the United States. J Clin Microbiol, 2015 53(5): p. 1537-1548.
52. Chen, Q., et al., Pathogenicity and pathogenesis of a United States porcine Deltacoronavirus cell culture isolate in 5-day-old neonatal piglets. Virology, 2015. 482: p. 51-59.
53. Li, G., et al., Full-Length Genome Sequence of Porcine Deltacoronavirus Strain USA/IA/2014/8734. Genome Announc, 2014. 2(2).
54. Marthaler, D., et al., Rapid detection, complete genome sequencing, and phylogenetic analysis of porcine Deltacoronavirus. Emerg Infect Dis, 2014a. 20(8): p. 1347-50.
55. Marthaler, D., et al., Complete Genome Sequence of Strain SDCV/USA/Illinois121/2014, a Porcine Deltacoronavirus from the United States. Genome Announc, 2014b. 2(2).
56. Wang, L., B. Byrum, and Y. Zhang, Detection and genetic characterization of Deltacoronavirus in pigs, Ohio, USA, 2014. Emerg Infect Dis, 2014. 20 (7): p. 1227-30.
57. Jung, K., et al., Pathogenicity of 2 porcine Deltacoronavirus strains in gnotobiotic pigs. Emerg Infect Dis, 2015. 21(4): p. 650-4.
58. Lee, S. and C. Lee, Complete Genome Characterization of Korean Porcine Deltacoronavirus Strain KOR/KNU14-04/2014. Genome Announc, 2014. 2(6).
59. Dong, N., et al., Porcine Deltacoronavirus in Mainland China. Emerg Infect Dis, 2015. 21(12): p. 2254-5.
60. Song, D., et al., Newly Emerged Porcine Deltacoronavirus Associated With Diarrhoea in Swine in China: Identification, Prevalence and Full-Length Genome Sequence Analysis. Transbound Emerg Dis, 2015. 62(6): p. 575-80.
61. Ma, Y., et al., Origin, evolution, and virulence of porcine Deltacoronaviruses in the United States. MBio, 2015. 6(2): p. e00064.
62. Vitosh-Sillman, S., et al. Histopathological and immunohistochemical characterization of pigs experimentally infected with porcine Deltacoronavirus. in American Association of Swine Veterinarians. 2015. Orlando, FL.
63. Ma, Y., et al., Origin, evolution, and virulence of porcine Deltacoronaviruses in the United States. MBio, 2015. 6(2).
64. ISERPD 2015. The 7^{th} Annual International Symposium on Emerging and Re-emerging Pig Diseases. Kyoto, Japan June 21-24, 2015. (See emerging2015.com/; emerging2015.com/pdf/ISERPD2015_Oral_Presentations_v8.pdf; and emerging2015.com/pdf/ISERPD2015_Poster_Presentations_v5.pdf, accessed November 4, 2016).

What is claimed is:

1. An immunogenic composition comprising an antigen of a porcine epidemic diarrhea virus (PEDV) and an adjuvant, wherein the PEDV comprises: (i) a polynucleotide having at least 99% sequence identity to SEQ ID NO: 32 or 36; (ii) a polynucleotide having at least 99% sequence identity to a RNA complement of SEQ ID NO: 29 or 33; and/or (iii) a spike protein encoded by a nucleic acid sequence having at least 90% sequence identity to SEQ ID NO: 30, 34, 46, or 52.

2. The immunogenic composition of claim 1, wherein the antigen is a chemically inactivated whole PEDV.

3. The immunogenic composition of claim 1, wherein the antigen is a whole PEDV chemically inactivated with ethylenimine, binary ethylenimine, acetylethylenimine, or any combinations thereof.

4. The immunogenic composition of claim 1, wherein the antigen is a recombinant antigen.

5. The immunogenic composition of claim 4, wherein the recombinant antigen comprises:
(i) a PEDV spike protein having at least 90% sequence identity to SEQ ID NO:31, 35, 47 or 53; and/or (ii) a polynucleotide encoding the PEDV spike protein having at least 90% sequence identity to SEQ ID NO:31, 35, 47 or 53.

6. The immunogenic composition of claim 4, wherein the recombinant antigen comprises a M, E, or N protein of PEDV.

7. The immunogenic composition of claim 4, wherein the recombinant antigen is a recombinant vector, a recombinant PEDV spike protein, or a combination thereof.

8. The immunogenic composition of claim 1, wherein the adjuvant is an oil-in-water emulsion.

9. The immunogenic composition of claim 1, further comprising a pharmaceutically acceptable carrier and/or an excipient.

10. The immunogenic composition of claim 1, further comprising an antigen of a porcine Deltacoronavirus (PDCoV).

11. The immunogenic composition of claim 10, wherein the PDCoV comprises: (i) a polynucleotide having at least 99% sequence identity to SEQ ID NO: 2, 6, or 10; (ii) a polynucleotide having at least 99% sequence identity to a RNA complement of SEQ ID NO: 1, 5, or 9; and/or (iii) a spike protein encoded by a nucleic acid sequence having at least 90% sequence identity to SEQ ID NO: 3, 7, 11, 17, or 27.

12. The immunogenic composition of claim 11, wherein the antigen of the PDCoV is an inactivated whole PDCoV.

13. The immunogenic composition of claim 11, wherein the antigen of the PDCoV is a recombinant antigen comprising: (i) a PDCoV spike protein having at least 90% sequence identity to SEQ ID NO: 4, 8, 12, 18, or 28; and/or (ii) a polynucleotide encoding the PDCoV spike protein having at least 90% sequence identity to SEQ ID NO: 4, 8, 12, 18, or 28.

14. A method for reducing a pig's risk for developing clinical signs of disease associated with PEDV, comprising administering to the pig the immunogenic composition according to claim 1.

15. A method for reducing a pig's risk for developing clinical signs of disease associated with PEDV, comprising administering to the pig the immunogenic composition according to claim 11, and wherein the method reduces the pig's risk for developing clinical signs of disease associated with PEDV and PDCoV.

16. A method for producing an immunogenic composition comprising a recombinant antigen of a PEDV and an adjuvant, comprising:
producing the recombinant antigen in a host cell;
harvesting the recombinant antigen from the host cell; and
adding an oil-in-water emulsion-based adjuvant to the recombinant antigen,
wherein the recombinant antigen comprises: (i) a PEDV spike protein having at least 90% sequence identity to SEQ ID NO:31, 35, 47 or 53; and/or (ii) a polynucleotide encoding the PEDV spike protein having at least 90% sequence identity to SEQ ID NO:31, 35, 47 or 53.

17. The method of claim 16, wherein the recombinant antigen comprises a PEDV spike protein expressed by a recombinant baculovirus vector.

18. The method of claim 17, wherein the host cell is an insect cell.

19. An immunogenic composition comprising an antigen of a PDCoV and an adjuvant, wherein the PDCoV comprises: (i) a polynucleotide having at least 99% sequence identity to SEQ ID NO: 2, 6, or 10; (ii) a polynucleotide having at least 99% sequence identity to a RNA complement of SEQ ID NO: 1, 5, or 9; and/or (iii) a spike protein encoded by a nucleic acid sequence having at least 90% sequence identity to SEQ ID NO: 3, 7, 11, 17, or 27.

20. The immunogenic composition of claim 19, wherein the antigen is:
an inactivated whole PDCoV; or
a recombinant antigen comprising: (i) a PDCoV spike protein having at least 90% sequence identity to SEQ ID NO: 4, 8, 12, 18, or 28; and/or (ii) a polynucleotide encoding the PDCoV spike protein having at least 90% sequence identity to SEQ ID NO: 4, 8, 12, 18, or 28.


SEQUENCE LISTING (53 sequences)

SEQ ID NO: 1 - DNA, 25505 bp, synthetic construct
  misc_feature 1..25505: note = Porcine Deltacorona Virus Genome cDNA - NSVL
  source 1..25505: mol_type = other DNA
gcacctgaaaccaagacgcgtcagtagtagggactactggtgcagccatgatagattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgttgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaatactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcaatgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaacttacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctacaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcccttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctatttagacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcacacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgctttgactcatgagccatgcagttcttgtcaacggtgggttaattatgactgcacctgcggatctagtctgcagaattcggcttatttaaacgcgtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttatcatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgytcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgttaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtctacctacaccactgcattacacaaatctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccaaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagtattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaagattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccattgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaattaggggaaattaaattggcttatagct

SEQ ID NO: 2 - RNA, 25505 nt, Porcine Deltacorona Virus
  source 1..25505: mol_type = genomic RNA
gcacctgaaaccaagacgcgtcagtagtagggactactggtgcagccatgatagattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgttgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaatactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcaatgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaacttacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctacaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcccttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctatttagacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcacacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgctttgactcatgagccatgcagttcttgtcaacggtgggttaattatgactgcacctgcggatctagtctgcagaattcggcttatttaaacgcgtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttatcatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgytcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgttaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtctacctacaccactgcattacacaaatctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccaaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagtattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaagattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccattgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaattaggggaaattaaattggcttatagct

SEQ ID NO: 3 - DNA, 3459 bp, synthetic construct
  misc_feature 1..3459: note = Deltacorona Virus SPIKE antigen CDS (NSVL) cDNA
  source 1..3459: mol_type = other DNA
atgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtctacctacaccactgcattacacaaatctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccaaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagtattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaagattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatgg

SEQ ID NO: 4 - protein, 1153 aa, Porcine Deltacorona Virus
MTLFCLVRAKFADDLLDLLTFPGAHRFLHKPTRNSSSLYSRANNNFDVGVLPGYPTKNVNLFSPLTNSTLPINGLHRSYQPLMLNCLTKITNHTLSMYLLPSEIQTYSCGGAMVKYQTHDAVRIILDLTATDHISVEVVGQHGENYVFVCSEQSTYTTALHKSTFFSLNSELYCFTNNTYLGILPPDLTDFTVYRTGQFYANGYLLGTLPITVNYVRLYRGHLSANSAHFALANLTDTLITLTNTTISQITYCDKSVVDSIACQRSSHEVEDGFYSNPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIVFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVSLYDGEVEIPSAFSLSVQTEYLQVQAEQVIVDCPQYVCNGNSRCLQLLAQYTSACSKIEAALHSSAQLDSREIINMFQTSTQSLQLANITNFKGDYNFSSILTTRIGGRSAIEDLLFNKVVTSGLGTVDQDYKSCSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSSRYGFCGNGTHIFSLTQTAPNGIFFMHAVLVPNKFTRVNASAGICVDNTRGYSLQPQLILYQFNNSWRVTPRNMYEPRLPRQADFIQLTDCSVTFYNTTAANLPNIIPDIIDVNQTVSDIIDNLPTATPPQWDVGIYNNTILNLTVEINDLQERSKNLSQIADRLQNYIDNLNNTLVDLEWLNRVETYLKWPWYIWLAIALALIAFVTILITIFLCTGCCGGCFGCCGGCFGLFSKKKRYTDDQPTPSFKFKEW

SEQ ID NO: 5 - DNA, 25426 bp, synthetic construct
  misc_feature 1..25426: note = Porcine Deltacorona Virus BIVI-Isolate 2.0307 Genome cDNA
  source 1..25426: mol_type = other DNA
gattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgctgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaacctcctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaacactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaatccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcagtgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcgaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaaattacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctccaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcctttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctattttgacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcgcacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgccttgactcatgagccatgcagttcttgtcaacggtgggttaattatgactgcacctgcggatctagtctgcagaattcggcttatttaaacgagtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttaccatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaattcggtatttaacatcttacaggtggtttctgctaatgtagccaccttcttatcaacttccaccacgacacatcttaataaggacattgcggacttgcatcatagtctttatgaagatatttatcgtggtgactctaatgatatcactgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgctcaggagttacaggatggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacctacgctgtgcatacccattgtagctaacggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgctaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgtgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgatgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggatggttgttcgacgtgagattgctactcacattgctgaggtagctcctcacactgattatatatgctttgtcacttgggctcaccagcttgagctagcgacaatgcgctactttgtcaaactaggtatggaagagagatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgcggtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaatatcaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttctacttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttactttgtctcgttcgagcaaagtttgctgatgatctactcgacttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttgaccaacatggtgaaaattatgtgtttgtttgcagtgagcagtttaactacaccactgcattacacaattctaccttcttctcacttaattctgcgctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagttttactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccatcgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaatt

SEQ ID NO: 6 - RNA, 25426 nt, synthetic construct
  misc_feature 1..25426: note = Porcine Deltacorona Virus BIV-Isolate 2.0307 ssRNA + Genome
  source 1..25426: mol_type = other RNA
gattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgctgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaacctcctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaacactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaatccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcagtgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcgaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaaattacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctccaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcctttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctattttgacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcgcacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgccttgactcatgagccatgcagttcttgtcaacggtgggttaattatgactgcacctgcggatctagtctgcagaattcggcttatttaaacgagtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttaccatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaattcggtatttaacatcttacaggtggtttctgctaatgtagccaccttcttatcaacttccaccacgacacatcttaataaggacattgcggacttgcatcatagtctttatgaagatatttatcgtggtgactctaatgatatcactgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgctcaggagttacaggatggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacctacgctgtgcatacccattgtagctaacggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgctaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgtgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgatgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggatggttgttcgacgtgagattgctactcacattgctgaggtagctcctcacactgattatatatgctttgtcacttgggctcaccagcttgagctagcgacaatgcgctactttgtcaaactaggtatggaagagagatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgcggtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaatatcaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttctacttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttactttgtctcgttcgagcaaagtttgctgatgatctactcgacttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttgaccaacatggtgaaaattatgtgtttgtttgcagtgagcagtttaactacaccactgcattacacaattctaccttcttctcacttaattctgcgctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagttttactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccatcgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaatt

SEQ ID NO: 7 - DNA, 3480 bp, synthetic construct
  misc_feature 1..3480: note = Porcine Deltacorona Virus BIV-Isolate 2.0307 Spike Antigen cDNA
  source 1..3480: mol_type = other DNA
atgcagagagctctattgattatgaccttactttgtctcgttcgagcaaagtttgctgatgatctactcgacttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttgaccaacatggtgaaaattatgtgtttgtttgcagtgagcagtttaactacaccactgcattacacaattctaccttcttctcacttaattctgcgctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagttttactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatgg

SEQ ID NO: 8 - protein, 1158 aa, Porcine Deltacorona Virus
MQRALLIMTLLCLVRAKFADDLLDLLTFPGAHRFLHKPTRNSSSLYSRANNNFDVGVLPGYPTKNVNLFSPLTNSTLPINGLHRSYQPLMLNCLTKITNHTLSMYLLPSEIQTYSCGGAMVKYQTHDAVRIILDLTATDHISVEVVDQHGENYVFVCSEQFNYTTALHNSTFFSLNSALYCFTNNTYLGILPPDLTDFTVYRTGQFYANGYLLGTLPITVNYVRLYRGHLSANSAHFALANLTDTLITLTNTTISQITYCDKSVVDSIACQRSSHEVEDGFYSDPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVLLYDGEVEIPSAFSLSVQTEYLQVQAEQVIVDCPQYVCNGNSRCLQLLAQYTSACSNIEAALHSSAQLDSREIINMFQTSTQSLQLANITNFKGDYNFSSILTTRIGGRSAIEDLLFNKVVTSGLGTVDQDYKSCSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSSRYGFCGNGTHIFSLTQTAPNGIFFMHAVLVPNKFTRVNASAGICVDNTRGYSLQPQLILYQFNNSWRVTPRNMYEPRLPRQADFIQLTDCSVTFYNTTAANLPNIIPDIIDVNQTVSDIIDNLPTATPPQWDVGIYNNTILNLTVEINDLQERSKNLSQIADRLQNYIDNLNNTLVDLEWLNRVETYLKWPWYIWLAIALALIAFVTILITIFLCTGCCGGCFGCCGGCFGLFSKKKRYTDDQPTPSFKFK

SEQ ID NO: 9 - DNA, 25498 bp, synthetic construct
  misc_feature 1..25498: note = Porcine Deltacorona Virus BIV-Isolate 5.0327 Genome cDNA
  source 1..25498: mol_type = other DNA
ctttgagcacctgaaaccaagacgcgtcagtagtagggactactggtgcagccatgatagattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgcgcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgctgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgtggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaacactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcagtgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaaattacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctacaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcccttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctatttagacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcacacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgctttgactcatgagccatgcagttcttgtcaacggtgggttaattatgactgcacctgcggatctagtctgcagaattcggcttatttaaacgcgtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttaccatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactyggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgctcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgttaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaactctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcattttagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccatcgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaataacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaattaggggaaattaaattggcttat

SEQ ID NO: 10 - RNA, 25498 nt, Porcine Deltacorona Virus
  source 1..25498: mol_type = genomic RNA
ctttgagcacctgaaaccaagacgcgtcagtagtagggactactggtgcagccatgatagattggtgtcggtagagaactagcgaagctagggagataaaattatagactaatgctataatttttatctttagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgcgcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgctgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgtggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaacactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcagtgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaaattacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctacaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcccttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctatttagacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcacacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgctttgactcatgagccatgcagttcttgtcaacggtgggttaattatgactgcacctgcggatctagtctgcagaattcggcttatttaaacgcgtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttaccatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactyggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgctcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgttaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaactctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcattttagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccatcgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaataacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaattaggggaaattaaattggcttat

SEQ ID NO: 11 - DNA, 3471 bp, synthetic construct
  misc_feature 1..3471: note = Porcine Deltacorona Virus BIV-Isolate 5.0327 Spike Antigen cDNA
  source 1..3471: mol_type = other DNA
atgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaactctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcattttagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatgg

SEQ ID NO: 12 - protein, 1157 aa, Porcine Deltacorona Virus
MQRALLIMTLFCLVRAKFADDLLDLLTFPGAHRFLHKPTRNSSSLYSRANNNFDVGVLPGYPTKNVNLFSPLTNSTLPINGLHRSYQPLMLNCLTKITNHTLSMYLLPSEIQTYSCGGAMVKYQTHDAVRIILDLTATDHISVEVVGQHGENYVFVCSEQFNYTTALHNSTFFSLNSELYCFTNNTYLGILPPDLTDFTVYRTGQFYANGYLLGTLPITVNYVRLYRGHFSAHFALANLTDTLITLTNTTISQITYCDKSVVDSIACQRSSHEVEDGFYSDPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVSLYDGEVEIPSAFSLSVQTEYLQVQAEQVIVDCPQYVCNGNSRCLQLLAQYTSACSNIEAALHSSAQLDSREIINMFQTSTQSLQLANITNFKGDYNFSSILTTRIGGRSAIEDLLFNKVVTSGLGTVDQDYKSCSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSSRYGFCGNGTHIFSLTQTAPNGIFFMHAVLVPNKFTRVNASAGICVDNTRGYSLQPQLILYQFNNSWRVTPRNMYEPRLPRQADFIQLTDCSVTFYNTTAANLPNIIPDIIDVNQTVSDIIDNLPTATPPQWDVGIYNNTILNLTVEINDLQERSKNLSQIADRLQNYIDNLNNTLVDLEWLNRVETYLKWPWYIWLAIALALIAFVTILITIFLCTGCCGGCFGCCGGCFGLFSKKKRYTDDQPTPSFKFKEW

SEQ ID NO: 13 - DNA, 33 bp, synthetic construct
  misc_feature 1..33: note = P2967167A PDCoV SPIKE FORWARD PRIMER
  source 1..33: mol_type = other DNA
ggatccatgcagagagctctattgattatgacc

SEQ ID NO: 14 - DNA, 42 bp, synthetic construct
  misc_feature 1..42: note = P2967167B PDCoV SPIKE REVERSE PRIMER
  source 1..42: mol_type = other DNA
ggtcgagccgcctctgccaccaattctggtggttagtaagct

SEQ ID NO: 15 - DNA, 31 bp, synthetic construct
  misc_feature 1..31: note = P2967167C FC FORWARD PRIMER
  source 1..31: mol_type = other DNA
tggtggcagaggcggctcgaccaagaccaag

SEQ ID NO: 16 - DNA, 36 bp, synthetic construct
  misc_feature 1..36: note = P2968014E FC FORWARD PRIMER
  source 1..36: mol_type = other DNA
gcggccgcctacttgcccggcgtcttcgaaatggat

SEQ ID NO: 17 - DNA, 2718 bp, synthetic construct
  misc_feature 1..2718: note = PDCoV S1-IgG2a Fc CDS cDNA Expression Casette
  source 1..2718: mol_type = other DNA
atgcagagagctctattgattatgaccttactttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaactctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcctactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctcaggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcttactaaccaccagaattggtggcagaggcggctcgaccaagaccaagcccccctgtcctatttgcccggcatgcgagagtccgggtccgtccgtctttattttccccccaaagccaaaagatacgctgatgatcagccgcaccccccaggtcacatgcgtggttgtagatgtaagccaagaaaatcccgaagtgcagttttcctggtatgtcgatggcgtggaggttcacacagcacagacgcggccgaaggaagagcagtttaattccacgtaccgagtggtgagtgtgctgcctattcagcatcaggattggttgaacggaaaggagttcaagtgcaaggtgaacaacaaagacctgcccgcaccaatcactcgcataatcagcaaggctaagggacagacacgcgagccacaggtgtacaccctgccaccgcatgcagaagagctgagccgatccaaggtatccataacctgcctggtgatcggattctacccccccgatatcgacgtcgagtggcagaggaatggccagccggagccagagggcaactaccggaccacgcctccgcagcaggatgtcgatggtacatattttctgtacagcaaatttagcgtggacaaggccagttggcagggcggcggtatcttccagtgtgctgtcatgcacgaggcactccacaatcattacacccagaaatccatttcgaagacgccgggcaagtag

SEQ ID NO: 18 - protein, 905 aa, synthetic construct
  REGION 1..905: note = PDCoV S1-IgG2a Fc Amino Acid Sequence
MQRALLIMTLLCLVRAKFADDLLDLLTFPGAHRFLHKPTRNSSSLYSRANNNFDVGVLPGYPTKNVNLFSPLTNSTLPINGLHRSYQPLMLNCLTKITNHTLSMYLLPSEIQTYSCGGAMVKYQTHDAVRIILDLTATDHISVEVVGQHGENYVFVCSEQFNYTTALHNSTFFSLNSELYCFTNNTYLGILPPDLTDFTVYRTGQFYANGYLLGTLPITVNYVRLYRGHLSANSAYFALANLTDTLITLTNTTISQITYCDKSVVDSIACQRSSHEVEDGFYSDPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVSLYDGEVEIPSAFSLSVQTEYLQVQAEQVIVDCPQYVCNGNSRCLQLLAQYTSACSNIEAALHSSAQLDSREIINMFQTSTQSLQLANITNFKGDYNFSSLLTTRIGGRGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 19 - DNA, 22 bp, synthetic construct
  misc_feature 1..22: note = P290194A F Primer PDCoVS BD
  source 1..22: mol_type = other DNA
ggatccgccaccatggtatctg

SEQ ID NO: 20 - DNA, 21 bp, synthetic construct
  misc_feature 1..21: note = P290194B R Primer PDCoVS BD
  source 1..21: mol_type = other DNA
gcttaaatacatatttagtac

SEQ ID NO: 21 - DNA, 100 bp, synthetic construct
  misc_feature 1..100: note = O2967002 A Primer Gp64 signal sequence
  source 1..100: mol_type = other DNA
ggatccgccaccatggtatctgctattgttctttatgttttgcttgctgccgccgctcactcagctttcgcggatgatctactcgatttgctcaccttcc

SEQ ID NO: 22 - DNA, 63 bp, synthetic construct
  misc_feature 1..63: note = O2967002B AcNPV gp64 C-terminal tail coding sequence
  source 1..63: mol_type = other DNA
gcggccgcttagtactgacgattgcggtttctaagaaagattgttatgaggattgtcacaaat

SEQ ID NO: 23 - DNA, 28 bp, synthetic construct
  misc_feature 1..28: note = P2967002C F Primer PDCoVS BD
  source 1..28: mol_type = other DNA
gatgatctactcgatttgctcaccttcc

SEQ ID NO: 24 - DNA, 26 bp, synthetic construct
  misc_feature 1..26: note = P2967002D R Primer PDCoVS BD
  source 1..26: mol_type = other DNA
cgatggccatgtctctagagcaggat

SEQ ID NO: 25 - DNA, 25 bp, synthetic construct
  misc_feature 1..25: note = P2967002E F Primer PDCoVS BD
  source 1..25: mol_type = other DNA
tgtcctatggacctatcagcgtgtg

SEQ ID NO: 26 - DNA, 31 bp, synthetic construct
  misc_feature 1..31: note = P2968002F R Primer PDCoVS BD
  source 1..31: mol_type = other DNA
aagaaagattgttatgaggattgtcacaaat

SEQ ID NO: 27 - DNA, 3390 bp, synthetic construct
  misc_feature 1..3390: note = PDCoVS BD CDS Expression Cassette
  source 1..3390: mol_type = other DNA
atggtatctgctattgttctttatgttttgcttgctgccgccgctcactcagctttcgcggatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaactctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcctactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctcaggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcttactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttcttagaaaccgcaatcgtcagtactaa

SEQ ID NO: 28 - protein, 1129 aa, synthetic construct
  REGION 1..1129: note = PDCoVS BD Protein - expressed baculodisplay
MVSAIVLYVLLAAAAHSAFADDLLDLLTFPGAHRFLHKPTRNSSSLYSRANNNFDVGVLPGYPTKNVNLFSPLTNSTLPINGLHRSYQPLMLNCLTKITNHTLSMYLLPSEIQTYSCGGAMVKYQTHDAVRIILDLTATDHISVEVVGQHGENYVFVCSEQFNYTTALHNSTFFSLNSELYCFTNNTYLGILPPDLTDFTVYRTGQFYANGYLLGTLPITVNYVRLYRGHLSANSAYFALANLTDTLITLTNTTISQITYCDKSVVDSIACQRSSHEVEDGFYSDPKSAVRARQRTIVTLPKLPELEVVQLNISAHMDFGEARLDSVTINGNTSYCVTKPYFRLETNFMCTGCTMNLRTDTCSFDLSAVNNGMSFSQFCLSTESGACEMKIIVTYVWNYLLRQRLYVTAVEGQTHTGTTSVHATDTSSVITDVCTDYTIYGVSGTGIIKPSDLLLHNGIAFTSPTGELYAFKNITTGKTLQVLPCETPSQLIVINNTVVGAITSSNSTENNRFTTTIVTPTFFYSTNATTFNCTKPVLSYGPISVCSDGAIVGTSTLQNTRPSIVSLYDGEVEIPSAFSLSVQTEYLQVQAEQVIVDCPQYVCNGNSRCLQLLAQYTSACSNIEAALHSSAQLDSREIINMFQTSTQSLQLANITNFKGDYNFSSLLTTRIGGRSAIEDLLFNKVVTSGLGTVDQDYKSCSRDMAIADLVCSQYYNGIMVLPGVVDAEKMAMYTGSLTGAMVFGGLTAAAAIPFATAVQARLNYVALQTNVLQENQKILAESFNQAVGNISLALSSVNDAIQQTSEALNTVAIAIKKIQTVVNQQGEALSHLTAQLSNNFQAISTSIQDIYNRLEEVEANQQVDRLITGRLAALNAYVTQLLNQMSQIRQSRLLAQQKINECVKSQSSRYGFCGNGTHIFSLTQTAPNGIFFMHAVLVPNKFTRVNASAGICVDNTRGYSLQPQLILYQFNNSWRVTPRNMYEPRLPRQADFIQLTDCSVTFYNTTAANLPNIIPDIIDVNQTVSDIIDNLPTATPPQWDVGIYNNTILNLTVEINDLQERSKNLSQIADRLQNYIDNLNNTLVDLEWLNRVETYLKWPWYIWLAIALALIAFVTILITIFLRNRNRQY

SEQ ID NO: 29 - DNA, 27960 bp, synthetic construct
  misc_feature 1..27960: note = Complete Genome Nucleic Acid (cDNA) Sequence equivalent of the (+) single-stranded RNA genome of PEDV Isolate 1251-125-10 (125-10)
  source 1..27960: mol_type = other DNA
agactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttattttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctacaggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgt

SEQ ID NO: 30 - DNA, 4140 bp, synthetic construct
  misc_feature 1..4140: note = PEDV Isolate 1251-125-10 (125-10) Spike-protein cDNA Sequence
  source 1..4140: mol_type = other DNA
atgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttattttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtga

SEQ ID NO: 31 - protein, 1379 aa, porcine epidemic diarrhea virus
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSINNDVTIGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTTKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 32 - RNA, 27960 nt, porcine epidemic diarrhea virus
  source 1..27960: mol_type = genomic RNA
agactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttattttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctacaggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgt

SEQ ID NO: 33 - DNA, 27976 bp, synthetic construct
  misc_feature 1..27976: note = PEDV 1251-125-10 (125-10) passage30 Genomic cDNA
  source 1..27976: mol_type = other DNA
aattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgmtcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgwtaaggtggcagacgtggctaactctgagcctgaggatgacggtcttaatgtagctcctgaaacaaatgtagagtctgaagttgaggaagttgccgcaaccttgtcctttaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctcyacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttwatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatrtgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctaytaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttaytggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattccccaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgtttctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacractcagcgctaggcttgagtctgttgaagttaactctatgcttactatttctgaagaggctctacagttagctaccattagttcgtttaatggtgatggatataattttactaatgtgctgggtgtttctgtgtatgatcctgcaaggggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatcccagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattcaaagtcggtggcaggctttgtttagtctgcttttactcctggcgctataaaaatgcgctctttattatttttaatactacgacactttctttcctcaatggtaaagcagctttgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatgg

SEQ ID NO: 34 - DNA, 4161 bp, synthetic construct
  misc_feature 1..4161: note = PEDV 1251-125-10 (125-10) passage30 spike protein CDS sequence
  source 1..4161: mol_type = other DNA
atgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattccccaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgtttctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacractcagcgctaggcttgagtctgttgaagttaactctatgcttactatttctgaagaggctctacagttagctaccattagttcgtttaatggtgatggatataattttactaatgtgctgggtgtttctgtgtatgatcctgcaaggggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatcccagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtga

SEQ ID NO: 35 - protein, 1386 aa, porcine epidemic diarrhea virus
  SITE 838: note = misc_feature - Xaa can be any naturally occurring amino acid
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTIGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSPNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTTKPLEGVTDVSFMTLDVCTKYTIYGFKGEGIITLTNSSFLAGVYYTSVSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALXLSARLESVEVNSMLTISEEALQLATISSFNGDGYNFTNVLGVSVYDPARGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 36 - RNA, 27976 nt, synthetic construct
  misc_feature 1..27976: note = PEDV 1251-125-10 (125-10) passage30 spike protein genomic RNA
  source 1..27976: mol_type = other RNA
aattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgmtcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgwtaaggtggcagacgtggctaactctgagcctgaggatgacggtcttaatgtagctcctgaaacaaatgtagagtctgaagttgaggaagttgccgcaaccttgtcctttaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctcyacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttwatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatrtgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctaytaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttaytggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaataggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattccccaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgactaaaccacttgaaggtgtcacggacgtttcttttatgactctggatgtgtgtaccaagtatactatctatggctttaaaggtgagggtatcattacccttacaaattctagctttttggcaggtgtttattacacatctgtttctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacractcagcgctaggcttgagtctgttgaagttaactctatgcttactatttctgaagaggctctacagttagctaccattagttcgtttaatggtgatggatataattttactaatgtgctgggtgtttctgtgtatgatcctgcaaggggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatcccagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattcaaagtcggtggcaggctttgtttagtctgcttttactcctggcgctataaaaatgcgctctttattatttttaatactacgacactttctttcctcaatggtaaagcagctttgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatgg

SEQ ID NO: 37 - DNA, 24 bp, synthetic construct
  misc_feature 1..24: note = P1360110A PEDV 2a BD- FORWARD PRIMER N-terminal fragment
  source 1..24: mol_type = other DNA
gatgtcaccaggtgctcagctaac

SEQ ID NO: 38 - DNA, 32 bp, synthetic construct
  misc_feature 1..32: note = PEDV-S2-R PEDV 2a BD- REVERSE PRIMER N-terminal fragment
  source 1..32: mol_type = other DNA
ctatacatgtgaagcttctcagcgtcaacaac

SEQ ID NO: 39 - DNA, 26 bp, synthetic construct
  misc_feature 1..26: note = PEDV-S1-F PEDV 2a BD- FORWARD PRIMER C-terminal fragment
  source 1..26: mol_type = other DNA
catctgattctggacagttgttagcc

SEQ ID NO: 40 - DNA, 33 bp, synthetic construct
  misc_feature 1..33: note = P1360110B.PEDV 2a BD- Reverse PRIMER C-terminal fragment
  source 1..33: mol_type = other DNA
gaacactagtaatgacacaacaaagatgagaac

SEQ ID NO: 41 - DNA, 107 bp, synthetic construct
  misc_feature 1..107: note = O1360110C gp64 signal sequence (AcNPV) cDNA
  source 1..107: mol_type = other DNA
gatccgccaccatggtatctgctattgttctttatgttttgcttgctgccgccgctcactcagctttcgcggatgtcaccaggtgctcagctaacactaattttagg

SEQ ID NO: 42 - DNA, 68 bp, synthetic construct
  misc_feature 1..68: note = O1360110D gp64 C-terminal tail coding sequence (AcNPV) cDNA
  source 1..68: mol_type = other DNA
gcttaaatacatatttagtactgacgattgcggtttctgaacactagtaatgacacaacaaagatgag

SEQ ID NO: 43 - DNA, 21 bp, synthetic construct
  misc_feature 1..21: note = P290194B Gp64 Forward Primer
  source 1..21: mol_type = other DNA
gcttaaatacatatttagtac

SEQ ID NO: 44 - DNA, 26 bp, synthetic construct
  misc_feature 1..26: note = PEDV-S1-F Gp64 Reverse Primer
  source 1..26: mol_type = other DNA
catctgattctggacagttgttagcc

SEQ ID NO: 45 - DNA, 22 bp, synthetic construct
  misc_feature 1..22: note = P290194A (OE Primer)
  source 1..22: mol_type = other DNA
ggatccgccaccatggtatctg

SEQ ID NO: 46 - DNA, 4062 bp, synthetic construct
  misc_feature 1..4062: note = PEDVS 2a BD Coding Sequence Expression Casette
  source 1..4062: mol_type = other DNA
atggtatctgctattgttctttatgttttgcttgctgccgccgctcactcagctttcgcggatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctttgggtgctacccaagtaccctattattgttttcttaaagtggatacttacaactccactgtttataaattcttggctgttttacctccaaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttcagaaaccgcaatcgtcagtactaa

SEQ ID NO: 47 - protein, 1353 aa, synthetic construct
  REGION 1..1353: note = PEDVS 2a BD Amino Acid from Expression Casette
MVSAIVLYVLLAAAAHSAFADVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFRNRNRQY

SEQ ID NO: 48 - DNA, 22 bp, synthetic construct
  misc_feature 1..22: note = P290194A PEDV 2b BD- FORWARD PRIMER N-terminal fragment
  source 1..22: mol_type = other DNA
ggatccgccaccatggtatctg

SEQ ID NO: 49 - DNA, 25 bp, synthetic construct
  misc_feature 1..25: note = P3183131B PEDV 2b BD- Reverse PRIMER N-terminal fragment
  source 1..25: mol_type = other DNA
gtcgatgctatggtccaaaaaccag

SEQ ID NO: 50 - DNA, 22 bp, synthetic construct
  misc_feature 1..22: note = P3183131A PEDV 2b BD- FORWARD PRIMER C-terminal fragment
  source 1..22: mol_type = other DNA
ggatccgccaccatggtatctg

SEQ ID NO: 51 - DNA, 21 bp, synthetic construct
  misc_feature 1..21: note = P290194B PEDV 2b BD- REVERSE PRIMER C-terminal fragment
  source 1..21: mol_type = other DNA
gcttaaatacatatttagtac

SEQ ID NO: 52 - DNA, 4053 bp, synthetic construct
  misc_feature 1..4053: note = PEDVS 2b BD CDS cDNA
  source 1..4053: mol_type = other DNA
atggtatctgctattgttctttatgttttgcttgctgccgccgctcactcagctttcgcggatgtcactaggtgccagtccactattaacttcaggcggttcttttcaaaatttaatgtgcaggcacctgctgtcgttgtgttgggtggttatctacctagtatgaactcctctagctggtactgtggcacaggtcttgaaactgctagtggcgtgcatggtattttcctcagttacatcgattctggtcagggctttgagattggcatttcacaggagccgtttgatcctagtggttaccagctttatttacataaggccactaatggtaaccataatgctattgcacgactgcgcatttgccagtttccaaataataatacattgggccctactgttaatgatgttacaacaggtcgtaactgcctattcaacaaagccattccagcttatatgcaggatggaaaaaacatcgttgtcggcataacatgggacaatgatcgtgtcactgtttttgctgacaagatctatcatttttatctcaaaaatgattggtcccgtgttgcgacaagatgttacaataaaagaagttgtgctatgcaatatgtttatacacctacctactacatgcttaatgttactagtgcaggtgaggatggcatttattatgaaccatgtacagctaattgcagtggttacgctgccaatgtgtttgccactgattctaatggccacataccagaaggttttagttttaataattggtttcttttgtccaatgattccactttgttgcatggtaaggtggtttccaaccaacctttgttggtcaattgtcttttggccattcctaagatttatggactaggccaatttttctcattcaatcaaacgatggatggcgtttgtaatggagctgctgcgcagcgtgcaccagaggctctgaggtttaatattaatgacacctctgtcattcttgctgaaggctcaattgtacttcacactgctttaggaacaaatctttcttttgtttgcagtaattcttcagatcctcatttagctaccttcaccatacctctgggtgctacccaagtaccctattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttcagaaaccgcaatcgtcagtactaa

SEQ ID NO: 53 - protein, 1350 aa, synthetic construct
  REGION 1..1350: note = PEDVS 2b BD Expression Casetter Amino Acid Sequence
MVSAIVLYVLLAAAAHSAFADVTRCQSTINFRRFFSKFNVQAPAVVVLGGYLPSMNSSSWYCGTGLETASGVHGIFLSYIDSGQGFEIGISQEPFDPSGYQLYLHKATNGNHNAIARLRICQFPNNNTLGPTVNDVTTGRNCLFNKAIPAYMQDGKNIVVGITWDNDRVTVFADKIYHFYLKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLLAIPKIYGLGQFFSFNQTMDGVCNGAAAQRAPEALRFNINDTSVILAEGSIVLHTALGTNLSFVCSNSSDPHLATFTIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFRNRNRQY